SEQUENCE LISTING (313 sequences)

SEQ ID NO: 1 - DNA, 24 bp, Artificial Sequence
  Description of Artificial Sequence Synthetic primer
gagcggataacaatttcacacagg

SEQ ID NO: 2 - DNA, 48 bp, Artificial Sequence
  Description of Artificial Sequence Synthetic primer
tgaggagacggtgacctgggtcccctggccccagtagtcngartancg

SEQ ID NO: 3 - DNA, 51 bp, Artificial Sequence
  Description of Artificial Sequence Synthetic primer
tgaggagacggtgacctgggtccccyggccccagwmgtcatacysatttga

SEQ ID NO: 4 - DNA, 51 bp, Artificial Sequence
  Description of Artificial Sequence Synthetic primer
tgaggagacggtgacctgggtcccctggccccaggamscaaadkcacggag

SEQ ID NO: 5 - DNA, 43 bp, Artificial Sequence
  Description of Artificial Sequence Synthetic primer
agtaggatccgccacctcctgaggagaccgtgacctgggtccc

SEQ ID NO: 6 - DNA, 64 bp, Artificial Sequence
  Description of Artificial Sequence Synthetic primer
tcttggatccggcgggggaggtagtgggggtgggggctcagaggtgcagctggtggagtctggg

SEQ ID NO: 7 - DNA, 22 bp, Artificial Sequence
  Description of Artificial Sequence Synthetic primer
gccagggttttcccagtcacga

SEQ ID NO: 8 - DNA, 35 bp, Artificial Sequence
  Description of Artificial Sequence Synthetic primer prVHH(s)
cggtcgtctcactctgaggtgcagctggtggagtc

SEQ ID NO: 9 - DNA, 34 bp, Artificial Sequence
  Description of Artificial Sequence Synthetic primer prVHH(as)
cggtcgtctctggttctgaggacacggtgacctg

SEQ ID NO: 10 - protein, 126 aa, Lama glama
EVQLVESGGGLVQAGGSLGLSCAASGFTSDDYAIGWFRQAPGKEREGVSYISHEDGSTYVADSVRGRFTISYDYAKNTVYLQMNSLKPEDTAVYYCAAAILGNSVLWLWEYEYRYWGQGTQVTVSS

SEQ ID NO: 11 - protein, 126 aa, Lama glama
EVQLVESGGGLVQAGGSLRLSCAASGFTSDDYAIGWFRQAPGKEREGVSYISHEDGSTYVADSVRGRFTISYDYAKNTVYLQMNSLKPEDTAVYYCAAAILGNSVLWLWEYEYRYWGQGTQVTVSS

SEQ ID NO: 12 - protein, 126 aa, Lama glama
EVQLVESGGGLVQAGESLRLSCAASGFTSDDYAIGWFRQAPGKEREGVSYISHEDGSTYVADSVRGRFTISYDYAKNTVYLQMNSLKPEDTAVYYCAAAILGNSVLWLWEYEYRYWGQGTQVTVSS

SEQ ID NO: 13 - protein, 126 aa, Lama glama
EVQLVESGGGLVQPGGSLRLSCAASGFTSDDYAIGWFRQAPGKEREGVSYISHEDGSTYVADSVRGRFTISYDYAKNTVYLQMNSLKPEDTAVYYCAAAILGNSVLWLWEYEYRYWGQGTQVTVSS

SEQ ID NO: 14 - protein, 126 aa, Lama glama
EVQLVESGGGLVQAGGSLRLSCAASGFTSDDYAIGWFRQAPGKEREGVSYISHEDGSTYVADSVRGRFTISYDYAKNTVYLQMNSLKPDDTAVYYCAAAILGNSVLWLWEYEYRYWGQGTQVTVSS

SEQ ID NO: 15 - protein, 126 aa, Lama glama
EVQLVESGGGLVQAGGSLRLSCAASGFTSDDYAIGWFRQAPGKEREGVSYISHEDGSTYVADSVGGRFTISYDYAKNTVYLQMNSLKPDDTAVYYCAAAILGNSVLWLWEYEYRYWGQGTQVTVSS

SEQ ID NO: 16 - protein, 126 aa, Lama glama
EVQLVESGGGLVQAGGSLRLSCAASGFTSDDYAIGWFRQAPGKEREGVSYISHEDGSTYVADSVRGRFTISSDYAKNTVYLQMNSLKPEDTAVYYCAAAILGNSVLWLWEYEYRYWGQGTQVTVSS

SEQ ID NO: 17 - protein, 126 aa, Lama glama
EVQLVESGGGLVQAGGSLRLSCAASGFTSDDYAIGWFRQAPGKEREGVSYISLEDGSTYVADSVRGRFTISYDYAKNTVYLQMNSLKPEDSAVYYCAAAILGNSVLWLWEYDYRYWGQGTQVTVSS

SEQ ID NO: 18 - protein, 126 aa, Lama glama
EVQLVESGGGLVQAGGSLRLSCAASGFTSDDYAIGWFRRAPGKEREGVSYVSIEDGSTYTADSVKGRFTISSDNAKNTVYLQMNSLKPEDTAVYYCAAAILGDYVRWLQEYDYDYWGQGTQVTVSS

SEQ ID NO: 19 - protein, 126 aa, Lama glama
EVQLVESGGGLVQAGGSLRLSCAASGFTSDDYAIGWFRRAPGKEREGVSYISIEDGSTYTADSVKGRFTISSDNAKNTVYLQMNSLKPEDTAVYYCAAAILGDYVRWLQEYDYDYWGQGTQVTVSS

SEQ ID NO: 20 - protein, 126 aa, Lama glama
EVQLVESGGGLVQTGGSLRLSCAASGFTSDDHAIGWFRRAPGKEREGLSYISIEDGSTYTADSVKGRFTISSDNAKNTVYLQMNSLKPEDTAVYYCAAAILGDYVRWLQEYDYDYWGQGTQVTVSS

SEQ ID NO: 21 - protein, 126 aa, Lama glama
EVQLVESGGGLVQAGGSLRLSCAASGFTSDDYAIGWFRRAPGKEREGVSYISIEDGSTYSADSVKGRFTISSDYAKNTVYLQMNGLKPEDTAVYYCAAAILGDSVRWLQEYDYDYWGQGTQVTVSS

SEQ ID NO: 22 - protein, 126 aa, Lama glama
EVQLVESGGGLVQPGGSLRLSCAASGFTSDDYAIGWFRRAPGKEREGVSYISNEDGSTYHADPVKGRFTIYSDNAKRTVYLQMNSLKPEDTAVYYCAAAILGDYVRWLSEYDYNYWGQGTQVTVSS

SEQ ID NO: 23 - protein, 126 aa, Lama glama
EVQLVESGGGLVQAGGSLRLSCAASGFTSDDYAIGWFRRAPGKEREGVSYISIEDGSMYYANSVKGRFTISSDNAKKTVYLQMNSLKVEDTAIYYCAAAIFGDSVRWLSEYEYNYWGQGTQVTVSS

SEQ ID NO: 24 - protein, 126 aa, Lama glama
EVQLVESGGGLVQAGGSLRLSCAASGFTSDDYAIGWFRQAPGKEREGVSYISAEDGSTYNADSVKGRFTISSDNAKNTVYLQMNSLKPEDTAVYYCAAALLGDSVRWLSEYDYDYWGQGTQVTVSS

SEQ ID NO: 25 - protein, 126 aa, Lama glama
EVQLVESGGGLVQAGGSLRLSCAASGFTSDDYAIGWFRQAPGKEREGVSYISAEDGSTYNADSVKGRFTISSDNAKNTVYLQMNSLKPEDTGVYYCAAALLGASVRWLSEYDYDYWGQGTQVTVSS

SEQ ID NO: 26 - protein, 126 aa, Lama glama
EVQLVESGGGLVQAGGSLRLSCAASGFTSDDYAIGWFRQAPGKEREGVSYISNEDGSTYVADSVKGRFTISSDNAKNTVYLQMNSLKPEDTAAYYCAAAILGNSVRWLSEYDYDYWGQGTQVTVSS

SEQ ID NO: 27 - protein, 126 aa, Lama glama
EVQLVESGGGLVQAGGSLRLSCACSGFTSDDYAIGWFRQAPGKEREGVSYISREDGSRHYADSAKGRFTISSDYAKNTVYLQMNSLEPEDTAVYYCAAALLGDYVLWLPEYEYKYWGQGTQVTVSS

SEQ ID NO: 28 - protein, 126 aa, Lama glama
EVQLVESGGGLVQAGGSLRLSCVCSGFSSDEYAIGWFRQAPGKEREGVSYISREDGSRHYADSVKGRFTISTDYARNTVYLQMDSLKPEDTGVYYCAAALLGDYVLWLPEYPYNYWGQGTQVTVSS

SEQ ID NO: 29 - protein, 126 aa, Lama glama
EVQLVESGGGLVQAGGSLRLSCACSGFTSDDYAIGWFRQAPGKEREGVSYISREDGGRYYADSVKGRFTISSDNAKNTVYLQMNSLKPEDTAVYYCAAALLGDYVRWLPEYEYNYWGQGTQVTVSS

SEQ ID NO: 30 - protein, 126 aa, Lama glama
EVQLVESGGGLVQAGGSLRLSCAASGFTLDDYAIGWFRQAPGKEREKVSFMFTSDGSGWYADSVKGRFTISSNNAKNTVYLEMNRLKPEDTAVYYCAAAYAGELVLWLPDYDYNYWGQGTQVTVSS

SEQ ID NO: 31 - protein, 126 aa, Lama glama
EVQLVESGGGLVQAGGSLRLSCAVSGGTFDDYAIGWFRQAPGKEREGVSYIGCNDGSTYNAGSVKGRFTISCDYAKNTVYLQMNNLNSEDTAVYYCAAAAQWATIRWIQEYDYDYWGQGTQVTVSS

SEQ ID NO: 32 - protein, 126 aa, Lama glama
EVQLVESGGGLVQAGGSLRLSCAVSGGTFDDYAIGWFRQAPGKEREGVSYIGCNDGSTYNAGSVKGRFTISCDYAKNTVYLQMNNLKPEDTAVYYCAAAAQWATIRWIQEYDYDYWGQGTQVTVSS

SEQ ID NO: 33 - protein, 126 aa, Lama glama
EVQLVESGGGLVQAGGSLRLSCAVSGGTFDDYAIGWFRQAPGKEREGVSYIGCNDGSTYYANSVKGRFTISCDYAKNTVYLQMNNLKPEDTAVYYCAAAAQWATIRWIQEYDYDYWGQGTQVTVSS

SEQ ID NO: 34 - protein, 126 aa, Lama glama
EVQLVESGGGLVQAGGSLRLSCAVSGGTFDDYAIGWFRQAPGKEREGVSYIGCNDGSTYHADSVKGRFTISCDYAKNTVYLQMNNLKPEDTAVYYCAAAAQWATIRWIQEYYYDYWGQGTQVTVSS

SEQ ID NO: 35 - protein, 126 aa, Lama glama
EVQLVESGGGLVQAGGSLRLSCAVSGGTFDDYAIGWFPQAPGKEREGVSYIGCNDGSTYYADSVKGRFTISCDYPKNTVYLQMNHLKPEDTAAYYCAAAAQWATIRWIQQYHYDYWGQGTQVTVSS

SEQ ID NO: 36 - protein, 126 aa, Lama glama
EVQLVESGGGLVQAGGSLRLSCAVSGGTFDDYAIAWFRQAPGKEREGVSYIGCNDGSTYYAGSVKGRFTISCDNAKNTVYLQMNSLKPDDTAVYYCAAAAQWATIRWIQEYDYDYWGQGTQVTVSS

SEQ ID NO: 37 - protein, 126 aa, Lama glama
EVQLVESGGGLVQPGGSLRLSCAASGATFDDYAIGWFRQAPGKEREGVSYIGCNDGATYYAGSVKGRFTISCDYAKNTVYLQMNSLKPEDTAVYYCAAAAQWATIRWIHEYDYNIWGQGTQVTVSS

SEQ ID NO: 38 - protein, 126 aa, Lama glama
EVQLVESGGGLVQPGGSLRLSCAASGATFDDYAIGWFRQAPGKEREGVSYIGCNDGATYYSGSVKGRFTISCDYAKNTVYLQMNSLKPEDTAVYYCAAAAQWATIRWIHEYDYNIWGQGTQVTVSS

SEQ ID NO: 39 - protein, 126 aa, Lama glama
EVQLVESGGGLVQAGGSLRLSCAVSGATFDDYAIGWFRQAPGKEREGVSYIGCNDGSTYYAGSVKGRFTISCDYAKNTVYLQMNSLKPEDTAVYYCAAAAQWATIRWIQEYDYDTWGQGTQVTVSS

SEQ ID NO: 40 - protein, 126 aa, Lama glama
EVQLVESGGGLVQAGGSLRLSCAVSGATFDDYAIGWFRQAPGKEREGVSYIGCNDGSIYYAGSVKGRFTISCDYAKNTVYLQMNSLKPEDTAVYYCAAAAQWATIRWIQEYDYDTWGQGSQVTVSS

SEQ ID NO: 41 - protein, 126 aa, Lama glama
EVQLVESGGGLVQAGGSLRLSCAVSGATFDDYAIGWFRQAPGKEREGVSYIGCNDGSTYYAGSVKGRFTISCDYAKNTVYLQMNSLKPEDTAVYYCAAAAQWATIRWIHEYDYDTWGQGTQVTVSS

SEQ ID NO: 42 - protein, 126 aa, Lama glama
EVQLVESGGGLVQAGGSLRLSCAAFGFTFDDYAIGWFRQAPGKEREGVSYIGCNDYSTYYSDSVKGRFTISCDYAKNTVYLQMNNLKPEDTAVYYCAAAAQWATIRWIHEYDYNYWGQGTQVTVSS

SEQ ID NO: 43 - protein, 126 aa, Lama glama
EVQLVESGGGLVQAGGSLRLSCAASGFTFDDYAIGWFRQAPGKEREGVSYIGCNDGSTYIADSVKGRFTISCDYAENTVYLQMDSLKSEDTAVYYCAAAAQWATIRWIQEYDYDYWGQGTQVTVSS

SEQ ID NO: 44 - protein, 126 aa, Lama glama
EVQLVESGGGLVQTGGSLRLSCVASGGTLDDYAIGWFRQAPGKEREGVSYIGSNDGSTYYAGAVKGRFTISSDHAKNTVYLQMNSLKPEDTAVYYCAAAAQWGTIRWIHEYDYEYWGQGTQVTVSS

SEQ ID NO: 45 - protein, 126 aa, Lama glama
EVQLVESGGGLVQAGGSLRLSCAASGGTLYDYAIGWFRQAPGKEREGVAYLGASDGATYYADSVKGRFTISSDNAKNTVYLQMNSLKPEDTAVYYCAAAAQWATIRWIQEYDYDTWGQGTQVTVSS

SEQ ID NO: 46 - protein, 124 aa, Lama glama
EVQLVESGGGLVQAGGSLRLSCAASGFTFDDYGIAWFRQAPGKEREGLACITRSGYTTYYLDSVRGRFTISSDNAKNTVYLQMNSLNPEDTAVYYCAADEPLVVWNCNGDFGSWGQGTQVTVSS

SEQ ID NO: 47 - protein, 124 aa, Lama glama
EVQLVESGGGLVQAGGSLRLSCATSGFTFDDYGIAWFRQAPGKEREGLACITRSGYTTYYLDSVKGRFTISSDNAKNTVYLQMNSLNPEDTGVYYCAADEPLIVWNCNGDFGSWGQGTQVTVSS

SEQ ID NO: 48 - protein, 119 aa, Lama glama
EVQLVESGGGLVQPGGSLRLSCETSRHTLDHYAIGWFRQAPGKENEGVACISTSTYYADSVKGRFSISRDNAKNTVYLQMNSLKPEDTAVYYCATTGSGYCTLRAFASWGQGTQVTVSS

SEQ ID NO: 49 - protein, 119 aa, Lama glama
EVQLVESGGGLVQPGGSLRLSCETSRHTLDHYAIGWFRQAPGKENEGVACISTSTYYADSVKGRFSISRDNVKNTVYLQMNSLKPEDTAVYYCATTGSGYCTLRAFASWGQGTQVTVSS

SEQ ID NO: 50 - protein, 117 aa, Lama glama
AISSGDGFGRTLGGSLRLSCETSRHTLDHYAIGWFRQAPGKDNEGVACITTSTYYADSVKGPFSISRDNAKNTVYLQMNSLKPEDTAVYYCATTGSGYCTLRAFASWGQGTQVIVSS

SEQ ID NO: 51 - protein, 119 aa, Lama glama
RWQLVESGGGLVQPGGSLRLSCETSRHTLDHYAIGWFRQAPGKGGEGVACISTNTYYADSVKGRFSISRDNAKNTVYLQMNDLKPEDTAVYHCATTGSGYCTLRAFASWGQGTQVTVSS

SEQ ID NO: 52 - protein, 119 aa, Lama glama
EVQLVESGGGLVQPGGSLRLSCETSRHTLDHYAIGWFRQAPGKEGEGVACISTNTYYADSVKGRFSISRDNAKNTVYLQMNDLKPEDTAVYHCATTGSGYCTLRAFASWGQGTQVTVSS

SEQ ID NO: 53 - protein, 119 aa, Lama glama
EVQLVESGGGLVQPGGSLRLSCETSRHTLDHYAIAWFRQAPGKEGEGIACVSTSTYYADSVKGRFSISRGNAKNTVYLQMNDLKPEDTAVYHCATTGSGYCTLRAFASWGQGTQVTVSS

SEQ ID NO: 54 - protein, 119 aa, Lama glama
EVQLVESGGGLVQPGGSLRLSCETSRHTLDHYAIGWFRQAPGKEGEGVACISTSTYYADSVKGRFSISRDNAKNTVYLQMNSLKPEDTAVYYCATTGSGLCTLRAFASWGQGTQVTVSS

SEQ ID NO: 55 - protein, 120 aa, Lama glama
EVQLVESGGGLVQPGGSLRLSCAASQFTLESYAIGWFRQAPGKDSEGVACISSSTYYADSVKGRFTISRDNAKNTVYLQMESLKPEDTAVYHCATSGAGSYCTLRAFGSWGQGTQVTVSS

SEQ ID NO: 56 - protein, 120 aa, Lama glama; identical to SEQ ID NO: 55

SEQ ID NO: 57 - protein, 120 aa, Lama glama
EVQLVESGGGLVQPGGSLRLSCAASQSTLESYAIGWFRQAPGKDSEGVACISSSTYYADSVKGRFTISRDNAKNTVYLQMESLKPEDTAVYHCATSGAGSYCTLRAFGSWGQGTQVTVSS

SEQ ID NO: 58 - protein, 125 aa, Lama glama
EVQLVESGGGLVQPGGSLRLSCAASGFTLDGYAIGWFRQAPGKEREAISCITTRGTIYYADSVKGRVTISRDNAKNTVYLQMTSLKPEDTAVYLCAADGFDTPCVAGTDWGYDYEGQGTQVTVSS

SEQ ID NO: 59 - protein, 125 aa, Lama glama
EVQLVESGGSLVQPGGSLRLSCAASGFTLDGYAIGWFRQAPGKEREAISCITTRGTIYYADSVKGRVTISRDNAKNTVYLQMTSLKPEDTAVYLCAADGFDTPCVAGTDWGYDYEGQGTQVTVSS

SEQ ID NO: 60 - protein, 125 aa, Lama glama
EVQLVESGGGLVQPGGSLRLSCAASGFTLDGYAIGWFRQAPGKEREAISCITTRGTIYYADSVKGRVTISRDNAKNTVYLQMNSLKPEDTAVYLCAADGFDTPCVAGTDWGYDYEGQGTQVTVSS

SEQ ID NO: 61 - protein, 125 aa, Lama glama
EVQLVESGGGLVQPGGSLRLSCAASGFTLDGYAIGWFRQAPGKEREAISCITTAGTIYYADSVKGRVTISRDNAKNTVYLQMNSLKPEDTAVYLCAADGFDTPCVAGTDWGYDYEGQGTQVTVSS

SEQ ID NO: 62 - protein, 125 aa, Lama glama
EVQLVESGGGLVQPGGSLRLSCAASGFTLDGYAIGWFRQTPGKEREAISCITTAGTIYYADSVKGRVTISRDNAKNTVYLQMNSLKPEDTAVYLCAADGFDTPCVAGTDWGYDYEGQGTQVTVSS

SEQ ID NO: 63 - protein, 125 aa, Lama glama
EVQLVESGGGLVQPGGSLRLSCAASGFTLDGYAIGWFRQAPGKEREAISCITTAGTIYYADSVKGRVTISRDNAKNTVYLQMNSLKPEDTAVYLCAVDGFDTPCVAGTDWGYDYEGQGTQVTVSS

SEQ ID NO: 64 - protein, 125 aa, Lama glama
EVQLVESGGGLVQPGGSLRLSCAASGFTLDGYAIGWFRQAPGKEREAISCITTAGTIYYADSVKGRVTISRDNTKNMVYLQMNSLKPEDTAVYLCAADGFDTPCVAGTDWGYDYEGQGTQVTVSS

SEQ ID NO: 65 - protein, 125 aa, Lama glama
EVQLVESGGGLVQPGGSLRLSCVASGFTLDGYAIGWFRQAPGKEREAISCITTAGTIYYADSVKGRVTISRDNAKNTVYLQMNSLKPEDTAVYLCAADGFDTPCVAGTDWGYDYEGQGTQVTVSS

SEQ ID NO: 66 - protein, 125 aa, Lama glama
EVQLVESGGGLVQLGGSLRLSCAASGFTLDGYAIGWFRQAPGKEREAISCITTAGTIYYADSVKGRVTISRDNAKNTVYLQMNSLKPEDTAVYLCAADGFDTPCVAGTDWGYDYEGQGTQVTVSS

SEQ ID NO: 67 - protein, 125 aa, Lama glama
EVQLVESGGGLVQPGGSLRLSCAASGFTLDGYAIGWFRQAPGKEREAISCITTAGTIYYADSVKGRVTISRDNAKNTVYLQMNSLKPEDTAVYLCAADGFDTPCVAGTDWGYDYGGQGTQVTVSS

SEQ ID NO: 68 - protein, 125 aa, Lama glama
EVQLVESGGGLVQAGGSLRLSCAASGFTLDGYAIGWFRQAPGKEREAISCITTAGTIYYADSVKGRVTISRDNAKNTVYLQMNSLKPEDTAVYLCAADGFDTPCVAGTDWGYDYEGQGTQVTVSS

SEQ ID NO: 69 - protein, 126 aa, Lama glama
EVQLVESGGGLVQPGGSLTLSCVASGFTLDGYAIGWFRQAPGKEVESVSCITGRHGTIYYADSVMGRFTISRDNAKSTVYLQMNSLKPEDTAVYYCAADGFDTPCVAGTDWQYDYWGQGTQVTVSS

SEQ ID NO: 70 - protein, 126 aa, Lama glama
EVQLVESGGGLVQPGGSLTLSCVASGFTLDGYAIGWFRQAPGKEVESVSCITGRHGTIYYADSVMGRFTISRDNAKNTVYLQMNSLKPEDTAVYYCAADGFDTPCVAGTDWQYDYWGQGTQVTVSS

SEQ ID NO: 71 - protein, 126 aa, Lama glama
EVQLVESGGGLVQPGGSLTLSCVASGFTLDGYAIGWFRQAPGKEVEPVSCITGRHGTIYYADSVMGRFTISRDNAKNTVYLQMNSLKPEDTAVYYCAADGFDTPCVAGTDWQYDYWGQGTQVTVSS

SEQ ID NO: 72 - protein, 121 aa, Lama glama
EVQLVESGGGLVQAGGSLRLSCIVSESIFSRYAMGWFRQAPGKEREFVAGIGAVTHYGEFVKGRFTISRDSAKNTIYLQMSSLKPEDTAIYFCARSKNTYISYASNQYDVWGRGTQVTVSS

SEQ ID NO: 73 - protein, 121 aa, Lama glama
EVQLVESGGGLVQAGGFLRLSCELRGSIFNQYAMAWFRQAPGKEREFVAGMGAVPHYGEFVKGRFTISRDNAKSTVYLQMNSLKPEDTAIYFCARSKSTYISYNSNGYDYWGRGTQVTVSS

SEQ ID NO: 74 - protein, 120 aa, Lama glama
EVQLVESGGGLVQPGGSLRLSCAASGNIVSIDAAGWFRQAPGKQREPVATILTGGATNYADSVKGRFTISRDNAKNTVYLQMSSLKPEDTAVYYCYAPMIYYGGRYSDYWGQGTQVTVSS

SEQ ID NO: 75 - protein, 120 aa, Lama glama
EVQLVESGGGLVQPGGSLRLSCTASGNIVSIDAAGWFRQAPGKQREPVATILTGGTTNYADSVKGRFTISRDTAKNTVYLQMNSLKPEDTAVYYCYAPMIYYGGSYSDYWGQGTQVTVSS

SEQ ID NO: 76 - protein, 120 aa, Lama glama
EVQLVESGGGLVQAGGSLRLSCTASINAVSIDAAGWFRQAPGMQREPVATILTSGSSTYADSVKGRFTISRDSAKNTVYLQMNSLKPEDTAVYTCYAPMVYYSGSYSDYWGQGTQVTVSS

SEQ ID NO: 77 - protein, 120 aa, Lama glama
EVQLVESGGALVQAGRSLRLSCAASGNAFTIDAAAWYRQAPGKQREPVATILSGGTTNYADSVKGRFTISRDNVKNTVYLQMNSLKPEDTAVYYCYVPMVYYSGRYNDVWGQGTQVTVSS

SEQ ID NO: 78 - protein, 119 aa, Lama glama
EVQLVESGGGLVQAGGSLRLSCAASGNAFSIDAAAWYRAPGKQREPVATILTGGSTNYADSVKGRFTISRDNAKNTVYLQMNSLKPEDTAVYYCYVPMVYYSGRYSDYWGQGTQVTVSS

SEQ ID NO: 79 - protein, 120 aa, Lama glama
EVQLVESGGGLVQAGGSLRLSCAASGNAFSIDAAAWYRQAPGKQREPVATILTGGSTNYADSVKGRFTISRDNAKNLVYLQMNSLKPEDTAVYYCYVPMVYYSGRYSDYWGQGTQVTVSS

SEQ ID NO: 80 - protein, 120 aa, Lama glama
EVQLVESGGGLVQAGGSLRLSCAASGNAFSIDAAAWYRQAPGKQREPVATILTGGSTNYADSVKGRFTISRDNAKNTVYLQMNSLKPEDTAVYYCYVPMVYYSGRYSDYWGQGTQVTVSS

SEQ ID NO: 81 - protein, 119 aa, Lama glama
EVQLVESGGGLVQPGGSLRLSCAASGNIVSIDAAGWFRQAPGKQREPVATILTGGATNYADSVKGRFTISRDNAKNTVYLQMNSLKPEDTAVYYCYAPMIYYGGRYFDYWGQGTVTVSS

SEQ ID NO: 82 - protein, 120 aa, Lama glama
EVQLVESGGGSLQAGGSLRLSCAASGNIVSIDAAGWFRQAPGKQREPVATILTGGATNYADSVKGRFTISRDNAKNTVYLQMNSLKPEDTAVYYCYAPMIYYGGRYSDYWGQGTQVTVSS

SEQ ID NO: 83 - protein, 120 aa, Lama glama
EVQLVESGGGLVQPGGSLRLSCAASGNIVSIDAAGWFRQAPGKQREPVATILTGGATNYADSVKGRFTISRDNAENTVYLQMNSLKPEDTAVYYCYAPMIYYGGRYSDYWGQGTQVTVSS

SEQ ID NO: 84 - protein, 120 aa, Lama glama
EVQLVESGGGLVQPGGSLRLSCAASGNIVSIDAAGWFRQAPGKQREPVATILTGGAANYADSVKGRFTISRDNAKNTVYLQMNSLKPEDTAVYYCYAPMIYYGGRYSDYWGQGTQVTVSS

SEQ ID NO: 85 - protein, 120 aa, Lama glama
EVQLVESGGGLVQPGGSLRLSCAASGNIASIDAAGWFRQAPGKQREPVATILTGGATNYADSVKGRFTISRDNAKNTVYLQMNSLKPEDTAVYYCYAPMIYYGGRYSDYWGQGTQVTVSS

SEQ ID NO: 86 - protein, 120 aa, Lama glama
EVQLVESGGGLVQPGGSLRLSCAASGNIVSIDAAGWFRQAPGKQREPVATILTGGATNYADSVKGRFTISRDNAKNTVYLQMNSLKPEDTAVYYCYAPMIAYGGRYSDYWGQGTQVTVSS

SEQ ID NO: 87 - protein, 120 aa, Lama glama
EVQLVESGGGLVQPGGSLRLSCAASGNIVSIDAAGWFRQAPGKQREPVATILTGGATNYADSVKGRFTISRDNAKNTVYLQMNSLKPEDTAVYYCYAPMIYYGGAYSDYWGQGTQVTVSS

SEQ ID NO: 88 - protein, 120 aa, Lama glama
EVQLVESGGGLVQPGGSLRLSCAASGNIVSIDAAGWFRQAPGKQREPVATILTGGATNYADSVKGRFTISRDNAKNTVYLQMNSLKPEDTAVYYCYAPMIYYGGRASDYWGQGTQVTVSS

SEQ ID NO: 89 - protein, 120 aa, Lama glama
EVQLVESGGGLVQPGGSLRLSCAASGNIVSIDAAGWFRQAPGKQREPVATILTGGATNYADSVKGRFTISRDNAKNTVYLQMNSLKPEDTAVYYCYAPMIYYGGRYSDAWGQGTQVTVSS

SEQ ID NO: 90 - protein, 126 aa, Lama glama
EVQLVESGGGLVQPGGSLKLSCAASGAAFDDYAIGWFRQAPGKEREGVSYIGCYDGAQYYAGSVKGRFTISCDYAKNTVYLQMNSLKPEDTAVYYCAAAYQWSTIRWVHEYDYNHLGQGTQVTVSS

SEQ ID NO: 91 - protein, 126 aa, Lama glama
EVQLVESGGGLVQPGGSLKLSCAASGASFHDYAIGWFRQAPGKEREGVSYIGCDDGAQYYAGSVKGRFTISCDYAKNTVYLQMNSLKPEDTAVYYCAAAYQWDTIRWVHEYDYSVWGQGTQVTVSS

SEQ ID NO: 92 - protein, 126 aa, Lama glama
EVQLVESGGGLVQPGGSLKLSCAASGAGFNDYAIGWFRQAPGKEREGVSYIGCLDGAQYYAGSVKGRFTISCDYAKNTVYLQMNSLKPEDTAVYYCAAAEQWNTIRWVHEYDYGDWGQGTQVTVSS

SEQ ID NO: 93 - protein, 126 aa, Lama glama
EVQLVESGGGLVQPGGSLKLSCAASGAAFHDYAIGWFRQAPGKEREGVSYIGCTDGAQYYAGSVKGRFTISCDYAKNTVYLQMNSLKPEDTAVYYCAAADQWNTIRWVHEYDYVYWGQGTQVTVSS

SEQ ID NO: 94 - protein, 126 aa, Lama glama
EVQLVESGGGLVQPGGSLKLSCAASGAPFNDYAIGWFRQAPGKEREGVSYIGCADGAQYYAGSVKGRFTISCDYAKNTVYLQMNSLKPEDTAVYYCAAADQWTTIRWVHEYDYHVWGQGTQVTVSS

SEQ ID NO: 95 - protein, 126 aa, Lama glama
EVQLVESGGGLVQPGGSLRLSCAASGADFDDYAIGWFRQAPGKEREGVSYIGCDDGAQYYAGSVKGRFTISCDYAKNTVYLQMNSLKPEDTAVYYCAAANQWTTIRWVHEYDYDIWGQGTQVTVSS

SEQ ID NO: 96 - protein, 126 aa, Lama glama
EVQLVESGGGLVQPGGSLKLSCAASGANFADYAIGWFRQAPGKEREGVSYIGCDDGAQYYAGSVKGRFTISCDYAKNTVYLQMNSLKPEDTAVYYCAAATQWNTIRWVHEYDYVFWGQGTQVTVSS

SEQ ID NO: 97 - protein, 126 aa, Lama glama
EVQLVESGGGLVQPGGSLKLSCAASGASFMDYAIGWFRQAPGKEREGVSYIGCEDGAQYYAGSVKGRFTISCDYAKNTVYLQMNSLKPEDTAVYYCAAAKQWETIRWVHEYDYHNWGQGTQVTVSS

SEQ ID NO: 98 - protein, 125 aa, Artificial Sequence
  Description of Artificial Sequence Synthetic L81H1 polypeptide
EVQLVESGGGLVQAGGSLRLSCVLSGGTLSDAAMGWFRQPPGKEREFVAAISWSAGPTTHYADSVKGRFTISRDDAKNTVYLQMNSLKPEDTGVYYCAAQFRVGWVHLSNEYDYWGQGTQVTVSS

SEQ ID NO: 99 - protein, 122 aa, Artificial Sequence
  Description of Artificial Sequence Synthetic L82B1A polypeptide
EVQLVESGGGLVQAGDSLRLSCAASTRTFRNYNLGWFRQAPGKEREFVAAISWADYRTFYSDSAKGRFTISRDNTKNMLYLQMNSLKPEDTARYYCGAGYRDVRVDWQPAFWGQGTQVTVSS

SEQ ID NO: 100 - protein, 118 aa, Artificial Sequence
  Description of Artificial Sequence Synthetic L82B1D polypeptide
EVQLVESGGGLVQAGDSLRLSCTASGSQFSHTDMAWARQPPGKEREFVAINWSAGNTYYRDSVKGRFTISRDNAQNTVYLQMNSLKPEDTATYYCAARIGYDVDWNYWGQGTQVTVSS

SEQ ID NO: 101 - protein, 129 aa, Artificial Sequence
  Description of Artificial Sequence Synthetic L82B2A polypeptide
EVQLVESGGGLVQAGGSLRLSCTASGTTFFMYAMGWFRQAPGKEREFVAAISRFGDSLHSSDSVKGRFTISRDNTENTVYLQMNMVKPEDTAVYYCAAKVAYHRSGSRYYTNHTDYDAWGQGTQVTVSS

SEQ ID NO: 102 - protein, 133 aa, Artificial Sequence
  Description of Artificial Sequence Synthetic L82B3F polypeptide
EVQLVESGGGLVQAGGSLRLSCTVSGQTSTTDAVGWFRQAPGKEREFVSAISWSDNKLYYEDSVKGRFAISRDNAKDTVYLQMDSLKPEDTAVYYCAARNRPPDFLTNYFRVLYYREGAYDYWGQGTQVTVSS

SEQ ID NO: 103 - protein, 128 aa, Artificial Sequence
  Description of Artificial Sequence Synthetic L82B4A polypeptide
EVQLVESGGGLVQAGGSLRLSCAASGFSLDDYAIGWFRQAPGKEREGLSCRSGSDGSTYYADSVKGRFTFSSDNAKNTVYLQLNSLKPEDTAVYYCALITGSSYCLWRRILESEFDVWGQGTQVTVSS

SEQ ID NO: 104 - protein, 127 aa, Artificial Sequence
  Description of Artificial Sequence Synthetic L82B4E polypeptide
EVQLVESGGGVVQAGGSLRLSCAASGRTFSLYTMGWFRQAPGKEREFIAHMRRGGYTTYVADSVKDRFTISGDNAEATVYLQMNSLKSEDTAVYYCAAKLGLLVIGTAPTANDYDYWGQGTQVTVSS

SEQ ID NO: 105 - protein, 131 aa, Artificial Sequence
  Description of Artificial Sequence Synthetic L82B12A polypeptide
EVQLVESGGDSVQPGGSLRLSCAASGFAYGTLDDYTIGWFRQVPGKEREGVACIGHNDDTTYYADSVQGRFTISRDNAKNTVYLQMSSLKPEDTAVYLCASTSRARCARYSYNDRWFFDTWGQGTQVTVSS

SEQ ID NO: 106 - protein, 121 aa, Artificial Sequence
  Description of Artificial Sequence Synthetic L88B2A polypeptide
EVQLVESGGGLVQAGGSLRLSCVASGDIQFSWSSMAWFRQAPGKEREFVATISRSGSDVDYADSAKGRFTISRDNAKKMLYLQMNSLKPEDTATYYCAAARGLTAYEFQFWGQGTQVTVSS

SEQ ID NO: 107 - protein, 118 aa, Artificial Sequence
  Description of Artificial Sequence Synthetic L88FA3 polypeptide
EVQLVESGGGLVQAGDSLKLSCTASTPTFSTLAMAWFRQAPGKEREFVAGIVDGMDNYDYSVEGRFTISRDNRKNTLYLQMDSLRPEDTAVYYCAARRRYYSSKYEYWGQGTQVTVSS

SEQ ID NO: 108 - protein, 117 aa, Artificial Sequence
  Description of Artificial Sequence Synthetic L88FA5 polypeptide
EVQLVESGGGLVQAGGSLRLSCAASFSSDNMGWFRQAPGKEREFIAAINWNGGTIVYADSVKGRFTISRDNAKNTVYLQMNSLKPEDTAVYYCAADSPGVVLSWDYWGQGTQVTVSS

SEQ ID NO: 109 - protein, 119 aa, Artificial Sequence
  Description of Artificial Sequence Synthetic L89B1D polypeptide
EVQLVESGGGSVQAGDSLKLSCTASTPTFSNLAMAWFRQAPGREREFVSGIVGDDITNYADFAEGRFTISRDNSKNTLYLQMDSLRPEDTAVYVCAARRRYYSSRYEYWGQGTQVTVSS

SEQ ID NO: 110 - protein, 119 aa, Artificial Sequence
  Description of Artificial Sequence Synthetic L89B2C polypeptide
EVQLVESGGGLVQAGGSLRISCEYSGRTFSTHRMAWFRQAPGKDREFVASLKWSDGETTYADSVKGRFTISRENAKNSLYLQMNNLEPGDTAVYFCAAGTWWPPSYDYWGQGTQVTVSS

SEQ ID NO: 111 - protein, 133 aa, Artificial Sequence
  Description of Artificial Sequence Synthetic L89B6B polypeptide
EVQLVESGGGLVQTGGSLRLSCTASGQTSIIDAVGWFRQAPGKEREFVSAISWSDNNIYYEDSVKGRFTISRDNAKDTVYLQMDSLKPEDTAVYYCAARNRPPDFLTNYFRVLYYREGAYDYWGQGTQVTVSS

SEQ ID NO: 112 - protein, 123 aa, Artificial Sequence
  Description of Artificial Sequence Synthetic L89B12D  polypeptide
EVQLVESGGGLVQSGDSLRLSCTISGIFFDLYSMAWFRQAPGKEREFVAAISSVGGMTDYADSVKGRFTISRDNARQTVYLQMNSLKPEDTAVYSCAARAHREIVRSYGYNYWGQGTQVTVSS

SEQ ID NO: 113 - protein, 118 aa, Artificial Sequence
  Description of Artificial Sequence Synthetic L832G6 polypeptide
EVQLVESGGDLVQAGDSLRLSCVVSGSIRDNSVIGWFRQVPGKERKLFTYYYWSGTSTYFVEAVKGRFTISRDKSKNTVYLQMNNLKHEDTALYYCATTSEGIMIDISGQGTQVSFLS

SEQ ID NO: 114 - protein, 119 aa, Artificial Sequence
  Description of Artificial Sequence Synthetic L833H1 polypeptide
EVQLVESGGGLVQAGDSLRLSCAVSGSISGIKTIGWFRQFPGKERKLFGYYYWNGASTYLVDSVKGRFTISRDNAENTVYLQMNNLKPEDTAIYYCGAETEGHYDFEFWGQGTQVTVSS

SEQ ID NO: 115 - protein, 125 aa, Artificial Sequence
  Description of Artificial Sequence Synthetic L833H3 polypeptide
EVQLVESGGGLVQAGGSLRLSCVLSGGTFSDAAMGWFRQPPGKEREFVAAISWSAGPTTHYADSVKGRFTISRDDAKNTVYLQMNSLKPEDTGVYYCAAQFRVGWVHLSNEYDYWGQGTQVTVSS

SEQ ID NO: 116 - protein, 130 aa, Artificial Sequence
  Description of Artificial Sequence Synthetic L8Cb15 polypeptide
EVQLVESGGGAVQPGDSLRLACTASGVTLDASRVTLDVYSIGWFRQAPGNEREAVSCISSSDGSTYYADSVKGRFTISRDDAKNTVYLQMNSLKPEDTAVYYCASDWSCEIVLYATADYWGQGTQVTVSS

SEQ ID NO: 117 - protein, 121 aa, Artificial Sequence
  Description of Artificial Sequence Synthetic L8Fe2 polypeptide
EVQLVESGGGLVQAGDSLRLSCTLSGGTYGFTEYRIGWFRQAPGLEREFAASIAWHDDTYYADSVRGRFLISRDNAKNTVQLQMNSLKPEDTAVYYCAAGLPGSPRRYDYWGQGTQVTVSS

SEQ ID NO: 118 - protein, 121 aa, Artificial Sequence
  Description of Artificial Sequence Synthetic L8Fg12 polypeptide
EVQLVESGGGLVQAGGSLRLSCAAAVSATRINAFGWYRQAPGKQRELVATILSDGNTNYADSVKGRFTISRDNTRNTVYLQMNGLKPEDTAVYVCNAELYYSDYLPTQTAWGQGTQVTVSS

SEQ ID NO: 119 - protein, 129 aa, Artificial Sequence
  Description of Artificial Sequence Synthetic L8Fj19 polypeptide
EVQLVESGGGLVQAGGSVRLSCAASGGTFRSYSMGWFRQAPGKEREFVGRITWTGSTSYADFVKGRFTISRDNAENMMYLQMNSLKPEDTAIYYCARDRTGLRYLTAQAMRDTNEYEYWGQGTQVTVSS

SEQ ID NO: 120 - protein, 129 aa, Artificial Sequence
  Description of Artificial Sequence Synthetic L8Fo17 polypeptide
EVQLVESGGGLVQAGGSLRLSCAASGRSFRVYGMAWFRQAPGKEREFVGRITWTASTSYADSVKGRFTISRDSAMNVIYLQMNSLKPEDTAVYYCATDRVSARYLTAQAMRDTDEYDYWGQGTQVTVSS

SEQ ID NO: 121 - protein, 129 aa, Artificial Sequence
  Description of Artificial Sequence Synthetic L8Fp6 polypeptide
EVQLVESGGGLVQAGGSLRLSCAASGRTINTYATGWFRQAPGKEREFVARISWGADSTNYADSVKGRFTISKDNAKNTVYLQMNSLKPEDTAVYYCAADKPYRALNLERMHDSSEYDVWGQGTQVTVSS

SEQ ID NO: 122 - protein, 127 aa, Artificial Sequence
  Description of Artificial Sequence Synthetic L8Hi20 polypeptide
EVQLVESGGGLVQTGGSLTLSCATSEGTFSAYTMAWFRQAPGKEREFVGAIDWRFGNTYYADSVKGRFTISRDNARNTGYLQMNSLKPEDTAVYFCAADRYRSIPYPPRRDSVYEYWGQGTQVTVSS

SEQ ID NO: 123 - protein, 128 aa, Artificial Sequence
  Description of Artificial Sequence Synthetic L91A1 polypeptide
EVQLVESGGGLVQPGGSLRLSCAVSGFPLGYYAIGWFRQAPGKEREEVSCIVTSDGSTYYTDSVKGRFTISRDAAKNTVYLQMNNLNPEDTAVYYCAAGGTLVAVTDSCFIDYVMDYWGKGTLVTVSS

SEQ ID NO: 124 - protein, 122 aa, Artificial Sequence
  Description of Artificial Sequence Synthetic L91E2 polypeptide
EVQLVESGGGLVQAGGSLRLSCTTSGSIFSLSGMGWFRQAPGKQREPVAVITSGGATNYAPYAKGRFTISRDNAKNTVYLQMNSLKPGDTAVYYCALDSPDRSLGAPPFDYWGQGTQVTVSS

SEQ ID NO: 125 - protein, 128 aa, Artificial Sequence
  Description of Artificial Sequence Synthetic L92A11 polypeptide
EVQLVESGGGLVQPGGSLRLSCAVSGFPLGYYAIGWFRQAPGKEREEVSCIVTSDGSTYYTDSVKGRFTISRDNAKNTVYLQMNNLNPEDTAVYYCAAGGTIVAVTDSCFIDYVMDYWGKGTLVTVSS

SEQ ID NO: 126 - protein, 126 aa, Artificial Sequence
  Description of Artificial Sequence Synthetic L92D4 polypeptide
EVQLVESGGGSVQAGASLRLSCAASGRTFSGIDMAWYRQAPGNGRELVAVITSGGSTKYVDSVKGRFTIFRDNAKNTVALQMNSLKPEDTAVYYCAAVEESPFYSGTYYPKPGDYWGQGTQVTVSS

SEQ ID NO: 127 - protein, 127 aa, Artificial Sequence
  Description of Artificial Sequence Synthetic L93B4 polypeptide
EVQLVESGGGLVQPGGSLRLSCAVSGFPLGYYAIGWFRQAPGKEREEVSCIVTSDGSTYYTDSVKGRFTISRDAAKNTVYLQMNNLNPEDTAVYYCAAGGTLVAVTDCFIDYVMDYWGKGTLVTVSS

SEQ ID NO: 128 - protein, 121 aa, Artificial Sequence
  Description of Artificial Sequence Synthetic L91H3 polypeptide
EVQLVESGGGLVQPGGSLRLSCAASGSILDDANAMGWYRQTPGTERALVALITDSGATRYADSVKGRFTISRDNAKNTATLQMNSLKPEDTAVYYCNFREFGGWGTNIDHWGQGTQVTVSS

SEQ ID NO: 129 - protein, 126 aa, Artificial Sequence
  Description of Artificial Sequence Synthetic L93D3 polypeptide
EVQLVESGGGLVQPGGSLRLSCAASGFGLDYYAIGWFRQAPGKEREGVSCIDSSDGSTYYVDSAKGRFTISRDNAKNTVYLQMNNLKPEDTAVYYCAAHALICGSYWSNGVVFDTWGQGTQVTVSS

SEQ ID NO: 130 - protein, 138 aa, Artificial Sequence
  Description of Artificial Sequence Synthetic L93D9 polypeptide
EVQLVESGGGLVQAGGSLRLSCATSGFRFDDYDIGWFRQAPGKEREGVSCISRGDGMTYYADFVKGRFTISSDSAKNTVYLQMNSLKPEDTAVYYCAADPGRSVGWVRAWSSTAQTMGVPSPADFASRGQGTQVTVSS

SEQ ID NO: 131 - protein, 120 aa, Artificial Sequence
  Description of Artificial Sequence Synthetic L93E6 polypeptide
EVQLVESGGGLVQPGGSLRLSCAASGFTLDNYAIGWFRQVPGKEREGVSCISSSDGKTYYADSVKGRFTISRDNAKNTVYLQMNSLKPEDTAVYYCARETRCLGSRWMGGGQGTQVTVSS

SEQ ID NO: 132 - protein, 128 aa, Artificial Sequence
  Description of Artificial Sequence Synthetic L93F2 polypeptide
EVQLVESGGGLVQPGGSLRLSCAVSGFPLGYYAIGWFRQAPGKEREEVSCIVTSDGSTYYTNSVKGRFTISRDNAKNTVYLQMNNLNPEDTAVYYCAAGGTIVAVTDSCFIDYVMDYWGKGTLVTVSS

SEQ ID NO: 133 - protein, 125 aa, Artificial Sequence; identical to SEQ ID NO: 59
  Description of Artificial Sequence Synthetic L93F12 polypeptide

SEQ ID NO: 134 - protein, 123 aa, Artificial Sequence
  Description of Artificial Sequence Synthetic L93H9 polypeptide
EVQLVESGGGLVQAGGSLRLSCRASGNTGNVNAMGWYRQAPGKQRELVAAVSGGGKTNYADFVKGRFTISIDNAKIIVYLQMNNLKPEDTAVYYCNAGVMGTSGSYYLGEDNWGQGTQVTVSS

SEQ ID NO: 135 - protein, 125 aa, Artificial Sequence
  Description of Artificial Sequence Synthetic L94H7 polypeptide
EVQLVESGGGLVQPGGSLRLSCTASGFQLVYHDLGWFRQVPGKEREGISCISSTDGSTYYTDSVKGRFTISRDNAKNIVYLQMNSLKSEDTAVYYCAADWRNVCRLPLGTGFGSWGQGTQVTVSS

SEQ ID NO: 136 - protein, 125 aa, Artificial Sequence
  Description of Artificial Sequence Synthetic L95B10D polypeptide
EVQLVESGGGLVQAGGSLTLSCEASGTIFSINGQGWFRQAPGKQREPVAFITSGGMTNYADHVKGRFTISRDNAKNTVYLQMNSLKPEDTAVYYCAAEDSPPNYRCSGEWCFDYWGQGTQVTVSS

SEQ ID NO: 137 - protein, 124 aa, Artificial Sequence
  Description of Artificial Sequence Synthetic L911B1E  polypeptide
EVQLVESGGGLVQPGGSLRLSCAASGNIFGENTMNWYRQAPGKQRELVAGITNLQSAYYPDGVKYADSVKGRFTISRDNAKNAVYLQMNSLKPEDTAVYYCNIFEHRFPPATYWGQGTQVTVSS

SEQ ID NO: 138 - protein, 116 aa, Artificial Sequence
  Description of Artificial Sequence Synthetic L911B2E  polypeptide
EVQLVESGGGLVQPGGSLRLSCAASGPLFSISTMGWYRQAPGEQRELVASISSDGDTNYSDSVKDRFTISRDNAKNTVYLQMNSLKPEDTAVYFCYARKWILDTYWGQGTQVTVSS

SEQ ID NO: 139 - protein, 114 aa, Artificial Sequence
  Description of Artificial Sequence Synthetic L911B9A  polypeptide
EVQLVESGGGLVQAGGSLRLSCTASGVTIDAVGWYRQAPGKQREPVATITSGGSTNYADSVKGRFTISRDDAKNTVYLQMNSLKPEDTAVYYCFRPGGPYSDSWGQGTQVTVSS

SEQ ID NO: 140 - protein, 120 aa, Artificial Sequence
  Description of Artificial Sequence Synthetic L911B11E  polypeptide
EVQLVESGGGLVQPGGSLRLSCAASGSIFSMTGMGWYRQPPGKQRELVAAITTDGSTHYADSVKERFTISGDNAKKTLYLQMNSLKPEDTAVYYCYAPMIYDRGSYRDYWGQGTQVTVSS

SEQ ID NO: 141 - protein, 124 aa, Artificial Sequence
  Description of Artificial Sequence Synthetic L911F1B  polypeptide
EVQLVESGGGLVQPGGSLRLSCAASGNILGENTMNWYRQAPGKERELVAGVTNLRSTYYPDGVKYADSVKGRFTVSRDNAKNAVYLQMNSLKPEDTAVYYCNLFEHRFPPATYWGQGTQVTVSS

SEQ ID NO: 142 - protein, 122 aa, Artificial Sequence
  Description of Artificial Sequence Synthetic L911F12B  polypeptide
EVQLVESGGALVKAGGSLRLSCAVSGTSVSDNGMGWGRQAPGKQRELVAVLTSGGATNYAPSVKGRFTISRDNAKNAVYLQMNNLKPEDTAVYYCAADTAARSLYSDPFDVWGQGTQVTVSS

SEQ ID NO: 143 - protein, 123 aa, Artificial Sequence
  Description of Artificial Sequence Synthetic L922B2 polypeptide
EVQLVESGGGLVQAGGSLRLSCRASGNTGNVNAMGWYRQAPGKQRELVAAVSGGGKTNYADSVKGRFTISIDNAKIIAYLQMNSLKPEDTAVYYCNAGVMGTSGSYYPGDDNWGQGTQVTVSS

SEQ ID NO: 144 - protein, 123 aa, Artificial Sequence
  Description of Artificial Sequence Synthetic L922B4 polypeptide
EVQLVESGGGLVQAGGSLRLSCRASGNTGNVNAMGWYRQAPGKQRELVAAVSGRGKTNYADSVKGRFTISIDNAKIIVYLQMNNLKPEDTAVYYCNAGVMGTSGSYYLGEDNWGQGTQVTVSS

SEQ ID NO: 145 - protein, 123 aa, Artificial Sequence
  Description of Artificial Sequence Synthetic L922E1 polypeptide
EVQLVESGGGLVQAGGSLRLSCAASGNTGNVNAMGWYRQAPGKQRELVAGISSRGTTNYADSVKGRFTISLDSAKITAHLQMNRLKPEDTAVYYCNAGVMGSSGSYYPGEENWGQGTQVTVSS

SEQ ID NO: 146 - protein, 123 aa, Artificial Sequence
  Description of Artificial Sequence Synthetic L923A1 polypeptide
EVQLVESGGGLVQAGGSLRLSCRASGNTGNVNAMGWYRQAPGKQRELVAAVSGGGKTNYADSVKGRFTISIDNAKIIVYLQMNNLKPEDTAVYYCNAGVMGTSGSYYLGEDNWGQGTQVTVSS

SEQ ID NO: 147 - protein, 123 aa, Artificial Sequence
  Description of Artificial Sequence Synthetic L932E8 polypeptide
EVQLVESGGGLVQAGGSLRLSCETSGNTVDDYAMGWFRQAPGKEGEVVACISTSGYTDYADSGKGRFSISIDSNKMTLHLHMKRLKHADADLCYSTASVMCSSGTYYPGEENWREGTPVTVSS

SEQ ID NO: 148 - protein, 124 aa, Artificial Sequence
  Description of Artificial Sequence Synthetic L9Ab16 polypeptide
EVQLVESGGGLVQAGGSLRLSCAASGIIFGSKGMGWYRQAPGKQRELVATVTSGGDIKYADSVKGRFSISRDNAKNTVYLQMNSLKPEDTAVYYCAALFPLYGTGELTPLDDYWGQGTQVTVSS

SEQ ID NO: 149 - protein, 122 aa, Artificial Sequence
  Description of Artificial Sequence Synthetic L9Ab19 polypeptide
EVQLVESGGGLVQAGGSLRLSCVASGIIFSDSGMGWFRQAPGKQREPVAVITRGGSTNYADSVKGRFTISRDNAKNTVYLQMNNLKPEDTAVYICAADTFDRHLQAPPFDYWGQGTQVTVSS

SEQ ID NO: 150 - protein, 116 aa, Artificial Sequence
  Description of Artificial Sequence Synthetic L9Ad3 polypeptide
EVQLVESGGGLVQAGGSLRLSCTASESSFSLTSMGWYRQAPGKQREAVASIDGDGTTSYADSVKGRFTISRDNAKNTVNLQMNSLKPEDTAVYFCFARKWILDIYWGQGTQVTVSS

SEQ ID NO: 151 - protein, 120 aa, Artificial Sequence
  Description of Artificial Sequence Synthetic L9Ad13 polypeptide
EVQLVESGGGLVQPGGSLRLSCAAPKIFSTAPMNWFRQAPGKQRELVAGISSGGSTMYADSVKGRFTISRDSAKNTLDLQMNSLKPEDTAVYYCNAEWAPLGPRSRVDYWGQGTQVTVSS

SEQ ID NO: 152 - protein, 121 aa, Artificial Sequence
  Description of Artificial Sequence Synthetic L9Ad14 polypeptide
EVQLVESGGGWVQPGGSLGLSCTAGRSAFSINDMGWYRQPPGKQREVVAIITFDGSANYADSVKGRFTISRRNYNNTVYLQMNNLKPEDTAVYYCNADAAASPRGGYNEYWGQGTQVTVSS

SEQ ID NO: 153 - protein, 125 aa, Artificial Sequence
  Description of Artificial Sequence Synthetic L9Aj2 polypeptide
EVQLVESGGGLVQPGGSLRLSCAASGFTLDTYAIGWFRQAPGKEREGVSCISKIDEQRYYLDSVKGRFTISRDNAKNTVYLQMNSLKPEDTAVYYCATPRSQYYCHNGAIGYDRWGQGTQVTVSS

SEQ ID NO: 154 - protein, 129 aa, Artificial Sequence
  Description of Artificial Sequence Synthetic L9An7 polypeptide
EVQLVESGGGLVQSGGSLRLSCAASGFTLDYYTVGWFRQAPGKEREGVSCINSSDGRTLYADSVKGRFTISRDNAKNTVYLQMNSLKPEDTAVYYCAAEKPGLTSYYYPCPPPGEYDYSGQGTQVTVSS

SEQ ID NO: 155 - protein, 125 aa, Artificial Sequence
  Description of Artificial Sequence Synthetic L9Ao15 polypeptide
EVQLVESGGGLVQTGGSLRLSCAASGGTLSSYAMGWFRQAPGKEREFVAAITWSGTRTLYADSVKGRFTISRDNANNTVYLQMNSLEPEDTAVYYCAAHRHPYALVVDHKAFDYWGQGTQVTVSS

SEQ ID NO: 156 - protein, 126 aa, Artificial Sequence
  Description of Artificial Sequence Synthetic L9Ap11 polypeptide
EVQLVESGGGSVQAGGSLRLSCAASGSIFNINAMGWYRQARGKGRELVAVVTNGGTTTYTGSVKGRFTISRDNAKNTVYLQMNSLKLEDTAVYYCAGVEESPFHSGTYYPLPGDYWGQGTQVTVSS

SEQ ID NO: 157 - protein, 127 aa, Artificial Sequence
  Description of Artificial Sequence Synthetic L9Bb3 polypeptide
EVQLVESGGGLVQAGGSLRLSCAASGRTFGTYAMAWFRQAAGKEREFVGAISSWGGGRTYYPPSVQGRFTISRENAKNTVYLQLSSLKPEDTAVYYCAAKRDNCSAFGCYAGPYDYWGQGTQVTVSS

SEQ ID NO: 158 - protein, 130 aa, Artificial Sequence
  Description of Artificial Sequence Synthetic L9Bc6 polypeptide
EVQLVESGGGLVQAGGSLRLSCAASGFTFDDYAIGWFRQAPGKEREGVLCFTRSRGSTDGSTYYADSVKGRFTISSDNAKNTVYLQMNSLKPEDTAVYYCATDHYLRVGLKCRDYEYDYWGQGTQVTVSS

SEQ ID NO: 159 - protein, 121 aa, Artificial Sequence
  Description of Artificial Sequence Synthetic L9Bd8 polypeptide
EVQLVESGGGLVQPGGSLRLSCIASGFTLDYYAMGWFRQAPGKEREGVACISSTDRSTYYPDSVKGRFTISRDNAENTVYLQMNSLKPEDTAVYSCAVGWNPDCENGMDYWGKGTLVTVSS

SEQ ID NO: 160 - protein, 118 aa, Artificial Sequence
  Description of Artificial Sequence Synthetic L9Bd9 polypeptide
EVQLVESGGGLVQTGGSLRLSCVASGNIYSLTSTGWYRQAPGQQREWIATIRGDDSTEYANSVKGRFTISRENALKTVYLQMNSLKPEDTAIYYCAARTLPLGQPNYWGQGTQVTVSS

SEQ ID NO: 161 - protein, 123 aa, Artificial Sequence
  Description of Artificial Sequence Synthetic L9Be11 polypeptide
EVQLVESGGGLVQSGGSLRLSCAASGFTLDSYAIGWFRQAPGKEREGVSCISNSDGRTYYADSVKGRFTISRDSAKNTVYLQMNSLKPEDTAVYLCATEGHCHGTNWTRPHSVSQGTQVTVSS

SEQ ID NO: 162 - protein, 129 aa, Artificial Sequence
  Description of Artificial Sequence Synthetic L9Bf19 polypeptide
EVQLVESGGGLVQPGGSLRLSCTASGRSISSSAMGWFRQAPGKEREFVASVHWTDVSTWYADSVKGRFTISRDNAKNTVYLQMNSVKPEDTAVYHCAASRAFSPDSWRRYSDSDSYESWGQGTQVTVSS

SEQ ID NO: 163 - protein, 118 aa, Artificial Sequence
  Description of Artificial Sequence Synthetic L9Bj13 polypeptide
EVQLVESGGGLVQAGGSLRLSCAASGSISSLNTMAWYRQTPGQQREWVARITGAGSTTYADSVKGRFTISRDDALYTVYLQMTSLKPEDTAVYYCAARTLPLGADNWWGQGTQVTVSS

SEQ ID NO: 164 - protein, 127 aa, Artificial Sequence
  Description of Artificial Sequence Synthetic L9Bm10 polypeptide
EVQLVESGGGLVQPGGSLRLSCAASGFTLDDYTTGWFRQAPGKEREGVSCISSSDGGTWYADSVKGRFTISRDNAKNTVYLQMNSLKPEDAAVYYCAVDSTAGSSCPGPDFYAMDYWGKGTLVTVSS

SEQ ID NO: 165 - protein, 121 aa, Artificial Sequence
  Description of Artificial Sequence Synthetic L9Bp16 polypeptide
EVQLVESGGGLVQAGESLRLSCVASERAFGMNYMGWFRQSAGKQREQVAAISQSGDTNYADSVKGRFTISRDNAKNTLYLEMNSLRPEDTAVYYCYAGPSIGLVISGLGYWGQGTQVTVSS

SEQ ID NO: 166 - protein, 127 aa, Artificial Sequence
  Description of Artificial Sequence Synthetic L9Ca12 polypeptide
EVQLVESGGGLVQPGGSLRLSCAASGFTLENYAIAWFRQAPGKEREGVSCISSKDSNTYYADSVKGRFTISRDNAKNTVYLQMNSLKPEDTAVYYCAADRLFSCAIIGWDDPPFGSWGQGTQVTVSS

SEQ ID NO: 167 - protein, 118 aa, Artificial Sequence
  Description of Artificial Sequence Synthetic L9Ca13 polypeptide
EVQLVESGGGLVQAGGSLRLSCAASGSIFSINAMAWYRQAPGKQREWVARITGDGTTNYADSVKGRFTISRDNAKSTMYLQMNSLKPEDTAVYYCAARALPLGTDNYWGQGTQVTVSS

SEQ ID NO: 168 - protein, 123 aa, Artificial Sequence
  Description of Artificial Sequence Synthetic L9Cd12 polypeptide
EVQLVESGGGLVQAGGSLRLSCAASGRTFSRYAMGWFRRAPGKEREFVATISASGMSTYYKDSVKGRFTISRDNAQNTTWLQMNSLKPEDTAVYYCAAADDFLPRLPSSYTYWGQGTQVTVSS

SEQ ID NO: 169 - protein, 122 aa, Artificial Sequence
  Description of Artificial Sequence Synthetic L9Cf15 polypeptide
EVQLVESGGGLVQAGGSLRLSCVASGSTSSINAMGWYRQTPGKQREFLAVIENIGNTRYADSVLGRFTISRDNAKNTVYLQMNSLKPEDTAVYYCNANARTTPTSISTAWFWGQGTQVTVSS

SEQ ID NO: 170 - protein, 125 aa, Artificial Sequence
  Description of Artificial Sequence Synthetic L9Cm18 polypeptide
EVQLVESGGGLVQAGGSLRLSCAASGFTFDDYDIGWFRQAPGKEREAVSRISNRDGSTYYLDSVRGRFTASSDNAKNAVYLQMNNLKPEDTAVYYCAATTALVGMLLPAYEYDYWGQGTQVTVSS

SEQ ID NO: 171 - protein, 128 aa, Artificial Sequence
  Description of Artificial Sequence Synthetic L9Co22 polypeptide
EVQLVESGGGLVQPGGSMRPSCTGSGFTLDDYAIGWFRQAPGKEREGVSCISSTDSRTYYPDSVEGRFTISRDNAKNTVYLQMNSLKPEDTAVYYCATDVLECNYPGGTYYPLAFGSWGQGTQVTVSS

SEQ ID NO: 172 - protein, 127 aa, Artificial Sequence
  Description of Artificial Sequence Synthetic L9Cp5 polypeptide
EVQLVESGGGLVQAGGSLRLSCAASGRTFSSCAMGWFRQAPGKEREIVAAISWRGGSTWYADSVKGRFTISRDNAKNTVYLQMSNLKPEDTAVYYCAADETSGSYYYTVDIDEYDYWGQGTQVTVSS

SEQ ID NO: 173 - protein, 116 aa, Artificial Sequence
  Description of Artificial Sequence Synthetic L9Cp13 polypeptide
EVQLVESGGGLVQPGGSLRLSCAASGTAFSITSMGWYRQASGEQRELVARIDSDGSTNYADSVKGRFTISRDNAKNTVYLQMNSLKPEDTAVYYCNAKKWILETYWGQGTQVTVSS

SEQ ID NO: 174 - protein, 125 aa, Artificial Sequence
  Description of Artificial Sequence Synthetic L1719A9 polypeptide
EVQLVESGGGLVQAGDSLRLSCAASGRTFSFDARGWFRQAPGKEREFVAAINWSGSDTHYADSVKGRFTISRDNAKNTLYLQMNSLKPEDTAVYYCAAAFDYSGSYNSVGLWDYWGQGTQVTVSS

SEQ ID NO: 175 - protein, 125 aa, Artificial Sequence
  Description of Artificial Sequence Synthetic L1719A11 polypeptide
EVQLVESGGGLVQAGDSLRLSCAASGRTFSFDAKGWFRQAPGKEREFVAAINWSGSDTHYADSVKGRFTISRDNAKNTAYLQMNSLKPEDTAVYYCAAAFDYSGSYDSVGLWDYWGQGTQVTVSS

SEQ ID NO: 176 - protein, 125 aa, Artificial Sequence
  Description of Artificial Sequence Synthetic L1719A12 polypeptide
EVQLVESGGGLVQAGDSLRLSCAASGRTFSFDARGWFRQAPGKEREFVAAINWSGSDTHYADSVKGRFTISRDNAKNTAYLQMNSLKPEDTAVYYCAAAFDYSGSYASVGLWDYWGQGTQVTVSS

SEQ ID NO: 177 - protein, 125 aa, Artificial Sequence
  Description of Artificial Sequence Synthetic L1719B12 polypeptide
EVQLVESGGGLVQAGDSLRLSCAASGRTFSFDARGWFRQAPGKEREFVAAINWCGSDAHYADSVKGRFTISRDNAKNTAYLQMNSLKPEDTAVYFCAAAFDYSGSYNSVGLWDYWGQGTQVTVSS

SEQ ID NO: 178 - protein, 125 aa, Artificial Sequence
  Description of Artificial Sequence Synthetic L1719C1 polypeptide
EVQLVESGGGLVRHGDSLRLSCAASRRTTSVYATGWFRQAPGKEREFVAAISWSGGYTNYADSVKGRFTISRDNARNTVFLQMNSLKPEDTAVYYCAADRNPYRGSNNANAYDYWGQGTQVTVSS

SEQ ID NO: 179 - protein, 125 aa, Artificial Sequence
  Description of Artificial Sequence Synthetic L1719D10 polypeptide
EVQLVESGGGLVQAGDSLRLSCAASGRTFSFDARGWFRQAPGKEREFVAAINWSGSDTHYADSVKGRFTISRDNAKNTVYLQMNSLKPEDTAVYYCAAAFDYSGSYDSVGLWDYWGQGTQVTVSS

SEQ ID NO: 180 - protein, 125 aa, Artificial Sequence
  Description of Artificial Sequence Synthetic L1719E1 polypeptide
EVQLVESGGGLVRHGDSLRLSCLVSGRTTSVYATGWFRQAPGKEREFVAAISWSGGYTNYADSVKGRFTISRDNAKNTVFLQMNSLKPEDTAVYYCAADRNPYRGSNNANAYDYWGQGTQVTVSS

SEQ ID NO: 181 - protein, 125 aa, Artificial Sequence
  Description of Artificial Sequence Synthetic L1719E11 polypeptide
EVQLVESGGGWVQAGDSLRLSCAASGRTFSFDAKGWFRQAPGKEREFVAAINWSGSDTHYADSVKGRFTISRDNAKNTVYLQMNSLKPEDTAVYYCAAAFDYSGSYNSVGLWDYWGQGTQVTVSS

SEQ ID NO: 182 - protein, 125 aa, Artificial Sequence
  Description of Artificial Sequence Synthetic L1719E12 polypeptide
EVQLVESGGGWVQAGDSLRLSCAASGRTFSFDAKGWFRQAPGKEREFVAAINWSGSDTHYADSVKGRFTISRDNAKNTVYLQMNSLKPEDTAVYYCAAAFDYSGNYNSVGLWDYWGQGTQVTVSS

SEQ ID NO: 183 - protein, 125 aa, Artificial Sequence
  Description of Artificial Sequence Synthetic L1719F11 polypeptide
EVQLVESGGGLVQAGDSLRLSCAASGRTFSFDARGWFRQAPGKEREFVAAINWSGSDAHYADSVKGRFTISRDNAKNTAYLQMNSLKPEDTAVYFCAAAFDYSGSYNSVGLWDYWGQGTQVTVSS

SEQ ID NO: 184 - protein, 125 aa, Artificial Sequence
  Description of Artificial Sequence Synthetic L1719H9 polypeptide
EVQLVESGGGLVQAGDSLRLSCAASGRSFSFDARGWFRQAPGKEREFVAAINWSGSDTHYADSVKGRFTISRDNAKNTAYLQMNSLKPEDTAVYYCAAAFDYSGSYNSVGLWDYWGQGTQVTVSS

SEQ ID NO: 185 - protein, 125 aa, Artificial Sequence
  Description of Artificial Sequence Synthetic L1719H10 polypeptide
EVQLVESGGGLVQAGDSLRLSCAASGRTFSFDARGWFRQAPGKEREFVAAINWSGSDAHYADSVKGRFTISRDNAKNTAYLQMNSLKPEDTAAYFCAAAFDYSGSYNSVGLWDYWGQGTQVTVSS

SEQ ID NO: 186 - protein, 125 aa, Artificial Sequence
  Description of Artificial Sequence Synthetic L1720C1 polypeptide
EVQLVESGGGLVRHGDSLRLSCAASRRTTSVYATGWFRQAPGKEREFVAAISWSGGYTNYADSVKGRFTISRDNAKDTVFLQMNSLKPEDTAVYYCAADRNPYRGSNNANAYDYWGQGTQVTVSS

SEQ ID NO: 187 - protein, 125 aa, Artificial Sequence
  Description of Artificial Sequence Synthetic L1720E4 polypeptide
EVQLVESGGGLVRHGDSLRLSCLVSRRTTSVYATGWFRQAPGKEREFVAAISWSGGYTNYADSVKGRFTISRDNAKNTVFLQMNSLKPEDTAVYYCAADQNPYRGSNNANAYDYWGQGTQVTVSS

SEQ ID NO: 188 - protein, 125 aa, Artificial Sequence
  Description of Artificial Sequence Synthetic L1721A3 polypeptide
EVQLVESGGGLVRHGDSLRLSCAASRRTTSVYATGWFRQAPGKEREFVAAISWSGGYTNYADSVKGRFTISRDNAKNTVFLQMNSLKPEDTAVYYCAADRNPYRGSNNANAYDYWGQGTQVTVSS

SEQ ID NO: 189 - protein, 125 aa, Artificial Sequence
  Description of Artificial Sequence Synthetic L1721A5 polypeptide
EVQLVESGGGLVQAGDSLRLSCAASGRTFSFDAKGWFRQAPGKEREFVAAINWSGSDTHYADSVKGRFTISRDNAKNTVYLQMNSLKPEDTAVYYCAAAFDYSGSYDSVGLWDYWGQGTQVTVSS

SEQ ID NO: 190 - protein, 125 aa, Artificial Sequence
  Description of Artificial Sequence Synthetic L1721A8 polypeptide
EVQLVESGGGLVRHGDSLRLSCLVSRRTTSVYATGWFRQAPGKEREFVAAISWSGGYTNYADSVKGRFTISRDNARNTVFLQMNSLKPEDTAVYYCAADRNPYRGSNNANAYDYWGQGTQVTVSS

SEQ ID NO: 191 - protein, 120 aa, Artificial Sequence
  Description of Artificial Sequence Synthetic L1721H4 polypeptide
EVQLVESGGGLVQPGGSLRLSCAASGFTLDYYAIGWFRQAPGKEREGVSCVSSSDGSTYYADSVKGRFTISRDNAENTVYLQMNSLKPEDTAVYYCATEQSCTGSSWSGPGQGTRVTVSS

SEQ ID NO: 192 - protein, 125 aa, Artificial Sequence
  Description of Artificial Sequence Synthetic L1723A9 polypeptide
EVQLVESGGGLVQAGDSLRLSCAASKRTTSAYATGWFRQAPGKEREFVAAISYSGAYTNYADSVKGRFTISRDNAKNTVYLQMNSLKPEDTAVYYCAADRNPYRGSNNANAYDYWGQGTQVTVSS

SEQ ID NO: 193 - protein, 125 aa, Artificial Sequence
  Description of Artificial Sequence Synthetic L1723A10 polypeptide
EVQLVESGGGLVQAGDSLRLSCAASGRTFSFDARGWFRQAPGKEREFVAAINWSGSDAHYADSVKGRFTISRDNAKNTAYLRMNSLKPEDTAVYFCAAAFDYSGSYNSVGLWDYWGQGTQVTVSS

SEQ ID NO: 194 - protein, 123 aa, Artificial Sequence; identical to SEQ ID NO: 146
  Description of Artificial Sequence Synthetic L1723A11 polypeptide

SEQ ID NO: 195 - protein, 125 aa, Artificial Sequence
  Description of Artificial Sequence Synthetic L1723E5 polypeptide
EVQLVESGGGLVRHGDSLRLSCLVSRRTTSVYATGWFRQAPGKEREFVAAISWSGGYTNYADSVKGRFTISRDNAKNTVFLQMNSLKPEDTAVYYCAADRNPYRGSNNANAYDYWGQGTQVTVSS

SEQ ID NO: 196 - protein, 127 aa, Artificial Sequence
  Description of Artificial Sequence Synthetic L2319G7 polypeptide
EVQLVESGEGLVQAGDSLRLSCAASGRTISSYAMGWFRQAPGKEREFVAAVSMSGGRTLYADSVKGRFTISKDNTKNTVYLQMNSLKPEDTAVYYCAAKWGYFDVALYAQERDYNYWGQGTQVTVSS

SEQ ID NO: 197 - protein, 119 aa, Artificial Sequence
  Description of Artificial Sequence Synthetic L2319G11 polypeptide
EVQLVESGGGLVQPGGSLRLSCAASGSIFSFNAMAWYRQAPGKQRELVAAISSGGSTNYADSVKGRFTISRDNAKNTVYLQMNSLKPEDTADYYCNLWHTDFVSFYDYWGQGTQVTVSS

SEQ ID NO: 198 - protein, 124 aa, Artificial Sequence
  Description of Artificial Sequence Synthetic L2319H7 polypeptide
EVQLVESGGGLVQAGGSLRLSCAASGRTFSDDLISWFRQAPGKERDFVAAVTFYGEDTYYADSVKGRFTISRDNAKNTVYLQMDRLKSEDTAVYHCASSSRATVIQNPLGYDYWGQGTQVTVSS

SEQ ID NO: 199 - protein, 123 aa, Artificial Sequence
  Description of Artificial Sequence Synthetic L2320E9 polypeptide
EVLVESGGGLVQAGGSLRLSCAASGRTFSNDLISWFRQAPGKERDFVAGVAFYGDDTYYADSVKGRFTISRENAKNRVYLQMDNLKSEDTGVYYCASSSRASVIQNPLGYDYWGQGTKVTVSS

SEQ ID NO: 200 - protein, 124 aa, Artificial Sequence
  Description of Artificial Sequence Synthetic L2320F9 polypeptide
EVQLVESGGGLVQAGGSLRLSCAASGRTFSNDLISWFRQAPGKERDFVAAVAFYGDDTYYADSVKGRFTISRDNAKNTVYLQMDNLKSEDTAVYYCASSSRATVIQNPLGYDYWGQGTQVTVSS

SEQ ID NO: 201 - protein, 127 aa, Artificial Sequence
  Description of Artificial Sequence Synthetic L2321B7 polypeptide
EVQLVESGGGLVQAGDSLRLSCAASGRTISSYAMGWFRQAPGKEREFVAAVSMSGGRTLYADSVKGRFTISKDNTKNTVYLQMNSLKPEDTAVYYCAAKWGYFDVALYAQERDYNYWGQGTQVTVSS

SEQ ID NO: 202 - protein, 125 aa, Artificial Sequence
  Description of Artificial Sequence Synthetic L2321H6 polypeptide
EVQLVESGGGLVQAGGSLRLACAASGRTFSDYAVGWFRQAPGKERQIVAAISWGGGDTYYTNSVKGRFTISRDNTRNTVYLQMSSLKPEDTAVYYCAANRNRRGVVSAPSSYDYWGQGTQVTVSS

SEQ ID NO: 203 - protein, 122 aa, Artificial Sequence
  Description of Artificial Sequence Synthetic VLP_A14 polypeptide
EVQLVESGGGLVQAGGSLRLSCAASGFSFNDYAIGWFRQTPGKEREGVSCLSSSGMAHYADSVKGRFTIAYDNAKNTVYLQMNSLKPEDTAVYHCATHPFRCGNWRTVMGSWGQGTQVTVSS

SEQ ID NO: 204 - protein, 122 aa, Artificial Sequence
  Description of Artificial Sequence Synthetic VLP_B9 polypeptide
EVQLVESGGGLVQAGGSLRLSCETSGFTFDDYGIGWFRQAPGKGREGISCISGSGIAHYGDSVKGRFTISSDNAKNTVYLQMNSLKPEDTGLYYCATTPFRCGNWRTTMGSWGQGTQVTVSS

SEQ ID NO: 205 - protein, 119 aa, Artificial Sequence
  Description of Artificial Sequence Synthetic VLP3_B21 polypeptide
EVQLVESGGGLVQAGGSLRLSCEASTSMFSIRAATWYRQAPGKQRELVANIDSEGTTGYSDSVKGRFTISRDNTKKTVYLQMNSLKPEDTAVYSCNAVVTYNMLVYDSWGQGTQVTVSS

SEQ ID NO: 206 - protein, 511 aa, Human immunodeficiency virus type 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 207 - protein, 121 aa, Artificial Sequence
  Description of Artificial Sequence Synthetic L8Cj3 polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 208 - protein, 121 aa, Artificial Sequence; identical to SEQ ID NO: 72
  Description of Artificial Sequence Synthetic L833E1 polypeptide

SEQ ID NO: 209 - protein, 121 aa, Artificial Sequence
  Description of Artificial Sequence Synthetic L8i5 polypeptide
EVQLVESGGGLVQAGGFLRLSCEPRGSIFNQYAMAWFRQAPGKEREFVAGMGAVAHYGEFVKGRFTISRDNAKNTAYLQMNSLKPEDTAIYFCARSKSTYISYNSNGYDYWGRGTQVTVSS

SEQ ID NO: 210 - protein, 121 aa, Artificial Sequence
  Description of Artificial Sequence Synthetic J3r (for improved expression yield in yeast) polypeptide
DVQLQESGGGLVQAGGSLRLSCELRGSIFNQYAMAWFRQAPGKEREFVAGMGAVPHYGEFVKGRFTISRDNAKSTVYLQMNSLKPEDTAIYFCARSKSTYISYNSNGYDYWGQGTQVTVSS

SEQ ID NO: 211 - protein, 121 aa, Artificial Sequence
  Description of Artificial Sequence Synthetic 1_4H (family approach) polypeptide
EVQLVESGGGLVQAGGFLRLSCVLRGSIFNQYAMAWFRQAPGKEREFVAGMGAVPHYGEFVKGRFTISRDNAKSTVYLQMNSLKPEDTAIYFCARSKSTYISYNSNQYDFWGQGTQVTVSS

SEQ ID NO: 212 - protein, 121 aa, Artificial Sequence
  Description of Artificial Sequence Synthetic 2_1H (family approach) polypeptide
EVQLVESGGGLVQAGGSLRLSCVLRGSDFSRYAMGWFRQAPGKEREFVAGMGAVAHYGEFVKGRFTISRDNAKNAQYLQMNSLKPEDTAIYFCARSKSTYISYNSNEYDYWGRGTQVTVSS

SEQ ID NO: 213 - protein, 121 aa, Artificial Sequence
  Description of Artificial Sequence Synthetic 2_4B (family approach) polypeptide
EVQLVESGGGLVQAGGSLRLSCELRGSIFSQYAMGWFRQAPGKEREFVAGMGAVAHYGEFVKGRFTISRDDAKNTINLQMNSLKPEDTAIYFCARSKSTYISYNSNQYDFWGQGTQVTVSS

SEQ ID NO: 214 - protein, 121 aa, Artificial Sequence
  Description of Artificial Sequence Synthetic 2_5B (family approach) polypeptide
EVQLVESGGGLVQAGGSLRLSCVLRGSDFSRYAMGWFRQAPGKEREFVAGMGAVAHYGEFVKGRFTISRDNAKNAQYLQMNSLKPEDTAIYFCARSKSTYISYNSNQYDIWGQGTQVTVSS

SEQ ID NO: 215 - protein, 121 aa, Artificial Sequence
  Description of Artificial Sequence Synthetic 2_6D (family approach) polypeptide
EVQLVESGGGLVQAGGSLRLSCVVSENIFSRYAMGWFRQAPGKEREFVAGIGANTHYGEFVKGRFTISRDNAKNTIYLQMNSLKPEDTAIYFCARSKNTYISYSSNQYDDWGQGTQVTVSS

SEQ ID NO: 216 - protein, 121 aa, Artificial Sequence
  Description of Artificial Sequence Synthetic 2_10F (family approach) polypeptide
EVQLVESGGGLVQAGGSLRLSCELRGSIFSQYAMGWFRQAPGKEREFVAGMGAVAHYGEFVKGRFTISRDDAKNTINLQMNSLKPEDTAIYFCARSKSTYISYNSNGYDVWGQGTQVTVSS

SEQ ID NO: 217 - protein, 121 aa, Artificial Sequence
  Description of Artificial Sequence Synthetic 3_3F (family approach) polypeptide
EVQLVESGGGLVQAGGFLRLSCELRGSIFNQYAMAWFRQAPGKEREFVAGMGAVPHYGEFVKGRFTISRDNAKNSVYLQMNSLKVEDTAIYFCARSKSTYISYNSNGYDSWGRGTQVTVSS

SEQ ID NO: 218 - protein, 121 aa, Artificial Sequence
  Description of Artificial Sequence Synthetic 4_2B (family approach) polypeptide
EVQLVESGGGLVQAGGFLRLSCELRGSIFNQYAMAWFRQAPGKEREFVAGMGAVPHYGEFVKGRFTISRDNAKNTVYLQMNSLKPEDTAIYFCARSKSTYISYNSNRYDYWGRGTQVTVSS

SEQ ID NO: 219 - protein, 121 aa, Artificial Sequence
  Description of Artificial Sequence Synthetic 4_4C (family approach) polypeptide
EVQLVESGGGLVQAGGFLRLSCELRGSIFNQYAMAWFRQAPGKEREFVAGMGAVPHYGEFVKGRFTISRDNAKSTVYLQMNSLKPEDTAIYFCARSKSTYISYNSNQYDYWGQGTQVTVSS

SEQ ID NO: 220 - protein, 121 aa, Artificial Sequence
  Description of Artificial Sequence Synthetic 4_6A (family approach) polypeptide
EVQLVESGGGLVQAGGFLRLSCELRGSIFNQYAMAWFRQTPGKEREFVAGMGAVPHYGEFVKGRFTISRDNAKNTVYLQMNSLKPEDTAIYFCARSKSTYISYNSNRYDYWGRGTQVTVSS

SEQ ID NO: 221 - protein, 121 aa, Artificial Sequence
  Description of Artificial Sequence Synthetic 4_6H (family approach) polypeptide
EVQLVESGGGLVQAGGSLRLSCELRGSIFSRYAMGWFRQAPGKEREFVAGMGAVEHYGEFVKGRFTISRDNAKNTVHLQMNSLKSEDTAIYFCARSKSTYISYNSNRYDNWGRGTQVTVSS

SEQ ID NO: 222 - protein, 121 aa, Artificial Sequence
  Description of Artificial Sequence Synthetic 4_11G (family approach) polypeptide
EVQLVESGGGLVQAGGSLRLSCVVSGSIFNQHAMGWFRQAPGKGREFVSGLSGTEHYGDLVKGRFTISRDNAKNTIYLQMNNLKPEDTAIYFCARSKNTFISYSSNKYDFWGQGTQVTVSS

SEQ ID NO: 223 - protein, 121 aa, Artificial Sequence
  Description of Artificial Sequence Synthetic 5_7H (family approach) polypeptide
EVQLVESGGGLVQAGGFLRLSCELRGSIFNQYAMAWFRQAPGKEREFVAGMGAVPHYGEFVKGRFTISRDNAKNTVYLQMNSLKPEDTAIYFCARSKSTYISYNSNGYDYWGRGTQVTVSS

SEQ ID NO: 224 - protein, 121 aa, Artificial Sequence
  Description of Artificial Sequence Synthetic 5_10B (family approach) polypeptide
EVQLVESGGGLVQAGGSLRLSCELRGSIFSQYAMGWFRQAPGKEREFVAGMGAVAHYGEFVKGRFTISREDAKNTINLQMNSLKPEDTAIYFCARSKSTYISYNSNGYDFWGRGTQVTVSS

SEQ ID NO: 225 - protein, 121 aa, Artificial Sequence
  Description of Artificial Sequence Synthetic 9_4F (family approach) polypeptide
EVQLVESGGGLVQAGGSLRLSCVVSGSIFSQHAMGWFRQAPGKGREFVSGISGTTHYGDLVKGRFTISRDNAKNTIYLQMNSLKPEDTAIYFCARSKNTYISYSSNGYDIWGRGTQVTVSS

SEQ ID NO: 226 - protein, 121 aa, Artificial Sequence
  Description of Artificial Sequence Synthetic J3_1 (site directed mutagenesis) polypeptide
EVQLVESGGGLVQAGGFLRLSCAASGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 227 - protein, 121 aa, Artificial Sequence
  Description of Artificial Sequence Synthetic J3_2 (site directed mutagenesis) polypeptide
EVQLVESGGGLVQAGGFLRLSCELRGSIFSQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 228 - protein, 121 aa, Artificial Sequence
  Description of Artificial Sequence Synthetic J3_3 (site directed mutagenesis) polypeptide
EVQLVESGGGLVQAGGFLRLSCELRGSIFNQYAMAWFRQAPGKEREFVAAIGAVPHYGEFVKGRFTISRDNAKSTVYLQMSSLKPEDTAIYFCARSKSTYISYNSNGYDYWGRGTQVTVSS

SEQ ID NO: 229 - protein, 121 aa, Artificial Sequence
  Description of Artificial Sequence Synthetic J3_4 (site directed mutagenesis) polypeptide
EVQLVESGGGLVQAGGFLRLSCELRGSIFNQYAMAWFRQAPGKEREFVAGMGAVPHYADSVKGRFTISRDNAKSTVYLQMSSLKPEDTAIYFCARSKSTYISYNSNGYDYWGRGTQVTVSS

SEQ ID NO: 230 - protein, 121 aa, Artificial Sequence
  Description of Artificial Sequence Synthetic J3_5 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAVYFCARSKSTYISYNSNGYDYWGRGTQVTVSS

SEQ ID NO: 231 - protein, 121 aa, Artificial Sequence
  Description of Artificial Sequence Synthetic J3_6 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYYCARSKSTYISYNSNGYDYWGRGTQVTVSS

SEQ ID NO: 232 - protein, 121 aa, Artificial Sequence
  Description of Artificial Sequence Synthetic J3_7 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCAASKSTYISYNSNGYDYWGRGTQVTVSS

SEQ ID NO: 233 - protein, 121 aa, Artificial Sequence
  Description of Artificial Sequence Synthetic J3_8 (site directed mutagenesis polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKNTYISYNSNGYDYWGRGTQVTVSS

SEQ ID NO: 234 - protein, 121 aa, Artificial Sequence
  Description of Artificial Sequence Synthetic J3_9 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYASNGYDYWGRGTQVTVSS

SEQ ID NO: 235 - protein, 121 aa, Artificial Sequence
  Description of Artificial Sequence Synthetic J3_10 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNEYDYWGRGTQVTVSS

SEQ ID NO: 236 - protein, 121 aa, Artificial Sequence
  Description of Artificial Sequence Synthetic J3_11 (site directed mutagenesis) polypeptide
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGQGTQVTVSS

SEQ ID NO: 237 - protein, 121 aa, Artificial Sequence
  Description of Artificial Sequence Synthetic J3_12 (site directed mutagenesis) polypeptide
EVQLVESGGGLVQAGGFLRLSCIYRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 238 - protein, 121 aa, Artificial Sequence
  Description of Artificial Sequence Synthetic J3_13 (site directed mutagenesis) polypeptide
EVQLVESGGGLVQAGGFLRLSCELRGRTFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 239 - protein, 122 aa, Artificial Sequence
  Description of Artificial Sequence Synthetic J3_CDR2_fill_1 (site directed mutagenesis) polypeptide
EVQLVESGGGLVQAGGFLRLSCELRGSIFNQYAMAWFRQAPGKEREFVAGMSGAVPHYGEFVKGRFTISRDNAKSTVYLQMSSLKPEDTAIYFCARSKSTYISYNSNGYDYWGRGTQVTVSS

SEQ ID NO: 240 - protein, 123 aa, Artificial Sequence
  Description of Artificial Sequence Synthetic J3_ CDR2_fill_2 (site directed mutagenesis) polypeptide
EVQLVESGGGLVQAGGFLRLSCELRGSIFNQYAMAWFRQAPGKEREFVAGMSWGAVPHYGEFVKGRFTISRDNAKSTVYLQMSSLKPEDTAIYFCARSKSTYISYNSNGYDYWGRGTQVTVSS

SEQ ID NO: 241 - protein, 124 aa, Artificial Sequence
  Description of Artificial Sequence Synthetic J3_ CDR2_fill_3 (site directed mutagenesis) polypeptide
EVQLVESGGGLVQAGGFLRLSCELRGSIFNQYAMAWFRQAPGKEREFVAGMSWSGAVPHYGEFVKGRFTISRDNAKSTVYLQMSSLKPEDTAIYFCARSKSTYISYNSNGYDYWGRGTQVTVSS

SEQ ID NO: 242 - protein, 120 aa, Artificial Sequence; identical to SEQ ID NO: 55
  Description of Artificial Sequence Synthetic L93E3 polypeptide

SEQ ID NO: 243 - protein, 120 aa, Artificial Sequence
  Description of Artificial Sequence Synthetic 3E3r (for improved expression yield in yeast) polypeptide
DVQLQESGGGLVQAGGSLRLSCAASQFTLESYAIGWFRQAPGKDSEGVACISSSTYYADSVKGRFTISRDNAKNTVYLQMNSLKPEDTAVYHCATSGAGSYCTLRAFGSWGQGTQVTVSS

SEQ ID NO: 244 - protein, 123 aa, Artificial Sequence
  Description of Artificial Sequence Synthetic 3E3CDR2 (3E3 with a three amino acid, back to germline, insertion in CDR2) polypeptide
QVQLQESGGGLVQAGGSLRLSCAASQFTLESYAIGWFRQAPGKDSEGVACISSSDGSTYYADSVKGRFTISRDNAKNTVYLQMNSLKPEDTAVYHCATSGAGSYCTLRAFGSWGQGTQVTVSS

SEQ ID NO: 245 - protein, 119 aa, Artificial Sequence; identical to SEQ ID NO: 48
  Description of Artificial Sequence Synthetic L94H5 polypeptide

SEQ ID NO: 246 - protein, 119 aa, Artificial Sequence; identical to SEQ ID NO: 49
  Description of Artificial Sequence Synthetic L9Bf11 polypeptide

SEQ ID NO: 247 - protein, 119 aa, Artificial Sequence; identical to SEQ ID NO: 51
  Description of Artificial Sequence Synthetic L93C3 polypeptide

SEQ ID NO: 248 - protein, 119 aa, Artificial Sequence; identical to SEQ ID NO: 52
  Description of Artificial Sequence Synthetic L9Cl22 polypeptide

SEQ ID NO: 249 - protein, 119 aa, Artificial Sequence; identical to SEQ ID NO: 53
  Description of Artificial Sequence Synthetic L922E2 polypeptide

SEQ ID NO: 250 - protein, 119 aa, Artificial Sequence; identical to SEQ ID NO: 54
  Description of Artificial Sequence Synthetic L94E4 polypeptide

SEQ ID NO: 251 - protein, 120 aa, Artificial Sequence; identical to SEQ ID NO: 57
  Description of Artificial Sequence Synthetic L94E5 polypeptide

SEQ ID NO: 252 - protein, 119 aa, Artificial Sequence
  Description of Artificial Sequence Synthetic 3E3_1 (family approach) polypeptide
EVQLVESGGGLVQPGGSLRLSCETSRHTLDHYAIGWFRQAPGKENEGVACISTSTYYADSVKGRFSISRDNAKNTVYLQMNSLKPEDTAVYYCATTGSGYCTLRAFDSWGQGTQVTVSS

SEQ ID NO: 253 - protein, 120 aa, Artificial Sequence
  Description of Artificial Sequence Synthetic 3E3_2 (family approach) polypeptide
EVQLVESGGGLVPPGGSLRLSCAASQFTLESYAIGWFRQAPGKDSEGVACISSSTYYADSVKGRFTISRDNAKNTVYLQMESLKPEDTAVYHCATSGAGSYCTLRAFASWGQGTQVTVSS

SEQ ID NO: 254 - protein, 119 aa, Artificial Sequence
  Description of Artificial Sequence Synthetic 3E3_3 (family approach) polypeptide
EVQLVESGGGLVQPGGSLRLSCETSRHTLDHYAIAWFRQAPGKEAEGVACISTSTYYADSVKGRFSISRDNAKNTVYLQMNSLKPEDTAVYHCATTGSGYCTLRAFASWGQGTQVTVSS

SEQ ID NO: 255 - protein, 120 aa, Artificial Sequence; identical to SEQ ID NO: 253
  Description of Artificial Sequence Synthetic 3E3_4 (family approach) polypeptide

SEQ ID NO: 256 - protein, 120 aa, Artificial Sequence
  Description of Artificial Sequence Synthetic 3E3_5 (family approach) polypeptide
EVQLVESGGGLVQPGGSLRLSCAASQFTLESYAIGWFRQAPGKDSEGVACISSSTYYADSVKGRFTISRDNAKNTVYLQMESLKPEDTAVYHCATSGAGSYCTLRAFESWGQGTQVTVSS

SEQ ID NO: 257 - protein, 119 aa, Artificial Sequence
  Description of Artificial Sequence Synthetic 3E3_6.1 (family approach) polypeptide
EVQLVESGGGLVQPGGSLRLSCETSRHTLDHYAIGWFRQAPGKEGEGVACISTSTYYADSVKGRFSISRDNAKNTVYLQMNSLKPEDTAVYYCATTGSGYCTLRAFGSWGQGTQVTVSS

SEQ ID NO: 258 - protein, 120 aa, Artificial Sequence
  Description of Artificial Sequence Synthetic 3E3_7 (family approach) polypeptide
EVQLVESGGGLVQPGGSLRLSCAASEFALQNYAIGWFRQAPGKDSEGVACISSSTYYADSVKGRFTISRDNAKNTVYLQMNSLKPEDTAVYYCATSGAGTYCTLRAFASWGQGTQVTVSS

SEQ ID NO: 259 - protein, 119 aa, Artificial Sequence
  Description of Artificial Sequence Synthetic 3E3_8.1 (family approach) polypeptide
EVQLVESGGGLVQPGGSLRLSCETSRHTLDHYAIGWFRQAPGKEGEGVACISTSTYYADSVKGRFSISRDNAKNTVYLQMNDLKPEDTAVYHCATTGSGYCTLRAFESWGQGTQVTVSS

SEQ ID NO: 260 - protein, 119 aa, Artificial Sequence
  Description of Artificial Sequence Synthetic 3E3_8.3 (family approach) polypeptide
EVQLVESGGGLVQPGGSLRLSCETSRHTLDHYAIGWFRQAPGKEGEGVACISTSTYYADSVKGRFSISRDNAKNTVYLQMNDLKPEDTAVYHCATTGSGYCTLREFASWGQGTQVTVSS

SEQ ID NO: 261 - protein, 120 aa, Artificial Sequence
  Description of Artificial Sequence Synthetic 3E3_8.2 (family approach) polypeptide
EVQLVESGGGLVPPGGSLRLSCAASQFTLESYAIGWFRQAPGKDIEGVACISSSTYYADSVKGRFTISRDNAKNTVYLQMESLKPEDTAVYHCATSGAGSYCTLRAFESWGQGTQVTVSS

SEQ ID NO: 262 - protein, 119 aa, Artificial Sequence
  Description of Artificial Sequence Synthetic 3E3_9 (family approach) polypeptide
EVQLVESGGGLVQPGGSLRLSCETSRHTLDHYAIAWFRQAPGKEGEGVACISTSTYYADSVKGRFSISRDNAKNTVYLQMNDLKPEDTALYHCATTGSGYCTLRAFASWGQGTQVTVSS

SEQ ID NO: 263 - protein, 119 aa, Artificial Sequence
  Description of Artificial Sequence Synthetic 3E3_10 (family approach) polypeptide
EVQLVESGGGLVQPGGSLRLSCETSRHTLDHYAIAWFRQAPGKEGEGIACVSTSTYYADSVKGRFSISRDNAKNTVYLQMNDLKPEDTAVYHCATTGSGYCTLRAFDSWGQGTQVTVSS

SEQ ID NO: 264 - protein, 119 aa, Artificial Sequence
  Description of Artificial Sequence Synthetic 3E3_11 (family approach) polypeptide
EVQLVESGGGLVQPGGSLRLSCETSKHTLDHYAIGWFRQAPGKEREGVSCISTNTYYSDSVKGRFTISRDNAKNTVYLQMNSLKPEDTAVYYCATTGSGLCTLRAFASWGQGTQVTVSS

SEQ ID NO: 265 - protein, 119 aa, Artificial Sequence
  Description of Artificial Sequence Synthetic 3E3_12 (family approach) polypeptide
EVQLVESGGGFVQPGGSLRLSCETSRHTLDHYAIGWFRQAPGKEYEGISCISTSTYYADSVKGRFTISRDNAKNTVYLQMNSLKPEDTAVYYCATTGSGLCTLRAFGSWGQGTQVTVSS

SEQ ID NO: 266 - protein, 119 aa, Artificial Sequence
  Description of Artificial Sequence Synthetic 3E3_13 (family approach) polypeptide
EVQLVESGGGLVQPGGSLRLSCETSRHTLDHYAIGWFRQAPGKEGEGVACISTSTYYADSVKGRFSISRDNAKNTVYLQMNDLKPEDTAVYHCATTGSGLCTLRAFASWGQGTQVTVSS

SEQ ID NO: 267 - protein, 119 aa, Artificial Sequence
  Description of Artificial Sequence Synthetic 3E3_14 (family approach) polypeptide
EVQLVESGGGLVQPGGSLRLSCETSRHTLDHYAIGWFRQAPGKEYEGISCISSSTYYADSVKGRFTISRDNAKNTVYLQMNSLKPEDTAVYYCATTGSGYCTLRAFESWGQGTQVTVSS

SEQ ID NO: 268 - protein, 119 aa, Artificial Sequence
  Description of Artificial Sequence Synthetic 3E3_15 (family approach) polypeptide
EVQLVESGGGLVQPGGSLRLSCETSRHTLDRYAIGWFRQVPGKEGEGVACISTSTYYADSVKGRFSISRDNAKNTVYLQMNDLKPEDTAVYHCATTGSGYCTLRAFDSWGQGTQVTVSS

SEQ ID NO: 269 - protein, 119 aa, Artificial Sequence
  Description of Artificial Sequence Synthetic 3E3_16 (family approach) polypeptide
EVQLVESGGGLVQPGGSLRLSCETSRHTLDHYAIGWFRQAPGKEAEGVACISTSTYYADSVKGRFSISRDNAKNTVYLQMNDLKPEDAAVYHCATTGSGYCTLRAFASWGQGTQVTVSS

SEQ ID NO: 270 - protein, 372 aa, Artificial Sequence
  Description of Artificial Sequence Synthetic L9Cj3-Fc(WT) polypeptide
MGWSCIILFLVATATGVHSEVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 115 aa, Artificial Sequence
  Description of Artificial Sequence Synthetic germline Vt polypeptide
QVQLVESGGGLVQAGGSLRLSCAASGRTFSSYAMGWFRQAPGKEREFVAAISWSGGSTYYADSVKGRFTISRDNAKNTVYLQMNSLKPEDTAVYYCAANAEYDYWGQCTQVTVSS

SEQ ID NO: 272 - protein, 121 aa, Artificial Sequence
  Description of Artificial Sequence Synthetic L8Cj3 polypeptide
EVQLVESGGGLVQAGGFLRLSCELRGSIFNQYAMGWFRQAPGKEREFVAGMGAVPHYGEFVKGRFTISRDNAKSTVYLQMSSLKPEDTAIYFCARSKSTYISYNSNGYDYWGRCTQVTVSS

SEQ ID NO: 273 - protein, 121 aa, Artificial Sequence
  Description of Artificial Sequence Synthetic L8i5 polypeptide
EVQLVESGGGLVQAGGFLRLSCEPRGSIFNQYAMGWFRQAPGKEREFVAGMGAVAHYGEFVKGRFTISRDNAKNTVYLQMNSLKPEDTAIYFCARSKSTYISYNSNGYDYWGRCTQVTVSS

SEQ ID NO: 274 - protein, 8 aa, Artificial Sequence
  Description of Artificial Sequence Synthetic Germline CDR2 peptide
SWSGGSTY

SEQ ID NO: 275 - protein, 5 aa, Artificial Sequence
  Description of Artificial Sequence Synthetic L8Cj3 CDR2 peptide
GAVPH

SEQ ID NO: 276 - protein, 6 aa, Artificial Sequence
  Description of Artificial Sequence Synthetic CDR2 fill 1 peptide
SGAVPH

SEQ ID NO: 277 - protein, 7 aa, Artificial Sequence
  Description of Artificial Sequence Synthetic CDR2 fill 2 peptide
SWGAVPH

SEQ ID NO: 278 - protein, 8 aa, Artificial Sequence
  Description of Artificial Sequence Synthetic CDR2 fill 3 peptide
SWSGAVPH

SEQ ID NO: 279 - protein, 5 aa, Artificial Sequence
  Description of Artificial Sequence Synthetic L93E3 CDR2 peptide
SSSTY

SEQ ID NO: 280 - protein, 8 aa, Artificial Sequence
  Description of Artificial Sequence Synthetic CDR2 fill peptide
SSSDGSTY

SEQ ID NO: 281 - protein, 15 aa, Artificial Sequence
  Description of Artificial Sequence Synthetic peptide
GGGGSGGGGSGGGGS

SEQ ID NO: 282 - protein, 15 aa, Artificial Sequence
  Description of Artificial Sequence Synthetic peptide
GGGSSGGGSSGGGSS

SEQ ID NO: 283 - protein, 6 aa, Artificial Sequence
  Description of Artificial Sequence Synthetic 6xHis tag
HHHHHH

SEQ ID NO: 284 - protein, 5 aa, Artificial Sequence
  Description of Artificial Sequence Synthetic peptide
GGGGS

SEQ ID NO: 285 - protein, 126 aa, Artificial Sequence
  Description of Artificial Sequence Synthetic polypeptide
XVQLVESGGGLVQXGXSLXLSCXCSGFXXDXXAIGWFRXAPGKEREXXSXXXXXDGXXXXAXXXXGRFTIXXXXAXXTVYLXMXXLXXXDXXXYYCAAAXXGXXVXWLXXYXYXYWGQGTQVTVSS

SEQ ID NO: 286 - protein, 126 aa, Artificial Sequence
  Description of Artificial Sequence Synthetic polypeptide
XVQLVESGGGLVQXGGSLXLSCXXXGXXXXDYAIXWFXQAPGKEREGVXYXGXXDXXXYXXXXVKGRFTISXDXXXNTVYLQMNXLXXXDTAXYYCAAAXQWXTIRWXXXYXYXXXGQGTQVTVSS

SEQ ID NO: 287 - protein, 124 aa, Artificial Sequence
  Description of Artificial Sequence Synthetic polypeptide
XVQLVESGGGLVQAGGSLRLSCAXSGFTFDDYGIAWFRQAPGKEREGLACITRSGYTTYYLDSVXGRFTISSDNAKNTVYLQMNSLNPEDTXVYYCAADEPLXVWNCNGDFGSWGQGTQVTVSS

SEQ ID NO: 288 - protein, 123 aa, Artificial Sequence
  Description of Artificial Sequence Synthetic polypeptide
XXQLXESGGGLVXXGGSLRLSCXXSXXXLXXYXIXWFRQXPGKXXEGXXCXXXXDGSTYYXDSVKGXFXISRXNXKNTVYLQMXXLKPEDXAXYXCATXGXGXXCTLRXFXSWGQGTQVXVSS

SEQ ID NO: 289 - protein, 126 aa, Artificial Sequence
  Description of Artificial Sequence Synthetic polypeptide
EVQLVESGGGLVQPGGSLTLSCVASGFTLDGYAIGWFRQAPGKERESVSCITGRHGTIYYADSVMGRFTISRDNAKSTVYLQMNSLKPEDTAVYYCAADGFDTPCVAGTDWQYDYWGQGTQVTVSS

SEQ ID NO: 290 - protein, 126 aa, Artificial Sequence
  Description of Artificial Sequence Synthetic polypeptide
XVQLVESGGXLVQXGGSLXLSCXASGFTLDGYAIGWFRQXPGKEXEXXSCITGXXGTIYYADSVXGRXTISRDNXKXXVYLQMXSLKPEDTAVYXCAXDGFDTPCVAGTDWXYDYXGQGTQVTVSS

SEQ ID NO: 291 - protein, 124 aa, Artificial Sequence
  Description of Artificial Sequence Synthetic polypeptide
XVQLXESGGGLVQAGGXLRLSCXXXXXXXXXXAMXWFRQXPGKXREFVXXXSWSXXXXHYXXXVKGRFTISRXXAKXXXXLQMXXLKXEDTAXYXCAXSKXTXISYXSNXYDXWGXGTQVTVSS

SEQ ID NO: 292 - protein, 120 aa, Artificial Sequence
  Description of Artificial Sequence Synthetic polypeptide
XVQLVESGGXLVQXGXSLRLSCXASXNXXXIDAAXWXRQAPGXQREPVATILXXGXXXYADSVKGRFTISRDXXKNTVYLQMNSLKPEDTAVYXCYXPMXXYXGXXXDYXGQGTQVTVSS

SEQ ID NO: 293 - protein, 113 aa, Artificial Sequence
  Description of Artificial Sequence Synthetic polypeptide
QVQLVESGGGLVQPGGSLRLSCAASGFTLDDYAIGWFRQAPGKEREGVSCISSSDGSTYYADSVKGRFTISRDNAKNTVYLQMNSLKPEDTAVYYCAADFGSWGQGTQVTVSS

SEQ ID NO: 294 - protein, 126 aa, Artificial Sequence
  Description of Artificial Sequence Synthetic polypeptide
XVQLVESGGGLVQPGGSLRLSCAASGXTXDDYAIGWFRQAPGKEREGVSXIXXXDGXTYYAXSVKGRFTISXDXAKNTVYLQMNSLKPEDTAVYYCAAAAQWATIRWIHEYDXXXWGQGTQVTVSS

SEQ ID NO: 295 - protein, 113 aa, Artificial Sequence
  Description of Artificial Sequence Synthetic polypeptide
QVQLVESGGGLVQPGGSLRLSCAASGFTLDDYAIGWFRQAPGKEREGVSCISSSDGSTYYADSVKGRFTISRDNAKNTVYLQMNSLKPEDTAVYYCAAEYDYWGQGTQVTVSS

SEQ ID NO: 296 - protein, 125 aa, Artificial Sequence
  Description of Artificial Sequence Synthetic polypeptide
XVQLVESGGGLVQXGGSLRLSCXXSGFXXDXYAIGWFRQAPGKEREGVXXISXXDGSXXYADSVKGRFTISXDXAXNTVYLQMXSLKPEDTXVYYCAAALLGDYVLWLPEYXYXWGQGTQVTVSS

SEQ ID NO: 297 - protein, 113 aa, Artificial Sequence
  Description of Artificial Sequence Synthetic polypeptide
QVQLVESGGGLVQPGGSLRLSCAASGFTLDYYAIGWFRQAPGKEREGVSCISSSDGSTYYADSVKGRFTISRDNAKNTVYLQMNSLKPEDTAVYYCATDFGSWGQGTQVTVSS

SEQ ID NO: 298 - protein, 119 aa, Artificial Sequence
  Description of Artificial Sequence Synthetic polypeptide
EVQLVESGGGLVQPGGSLRLSCETSRHTLDHYAIGWFRQAPGKENEGVACISTSTYYADSVKGRFTISRDNAKNTVYLQMNSLKPEDTAVYHCATTGSGYCTLRAFASWGQGTQVTVSS

SEQ ID NO: 299 - protein, 120 aa, Artificial Sequence
  Description of Artificial Sequence Synthetic polypeptide
EVQLVESGGGLVQPGGSLRLSCAASQSTLESYAIGWFRQAPGKDSEGVACISSSTYYADSVKGRFSISRDNAKNTVYLQMESLKPEDTAVYHCATSGAGSYCTLRAFGSWGQGTQVTVSS

SEQ ID NO: 300 - protein, 123 aa, Artificial Sequence
  Description of Artificial Sequence Synthetic polypeptide
XVQLVESGGGLVQPGGSLRLSCXXSXXTLXXYAIGWFRQAPGKXXEGVXCISXXDXXTYYADSVKGRFXISRDNAKNTVYLQMXXLKPEDTAVYXCATXGXGSXCTLRXFXSWGQGTQVTVSS

SEQ ID NO: 301 - protein, 113 aa, Artificial Sequence
  Description of Artificial Sequence Synthetic polypeptide
QVQLVESGGGLVQAGGSLRLSCAASGRTFSSYAMGWFRQAPGKEREFVAAISWSGGSTYYADSVKGRFTISRDNAKNTVYLQMNSLKPEDTAVYYCAAEYDYWGQGTQVTVSS

SEQ ID NO: 302 - protein, 121 aa, Artificial Sequence
  Description of Artificial Sequence Synthetic polypeptide
EVQLVESGGGLVQAGGSLRLSCIVSESIFSRYAMGWFRQAPGKEREFVAGIGAVTHYGEFVKGRFTISRDSAKNTIYLQMNSLKPEDTAIYFCARSKNTYISYASNQYDVWGRGTQVTVSS

SEQ ID NO: 303 - protein, 121 aa, Artificial Sequence
  Description of Artificial Sequence Synthetic polypeptide
EVQLVESGGGLVQAGGFLRLSCELRGSIFNQYAMGWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 304 - protein, 124 aa, Artificial Sequence
  Description of Artificial Sequence Synthetic polypeptide
XVQLVESGGGLVQAGGXLRLSCXXXXXXFXXYAMGWFRQAPGKEREFVAXXSWSGXXXXYXXXVKGRFTISRDXAKXTXYLQMXSLKPEDTAXYXCAXSKXTYISYXSNXYDYWGXGTQVTVSS

SEQ ID NO: 305 - protein, 126 aa, Artificial Sequence
  Description of Artificial Sequence Synthetic polypeptide
XVQLVESGGGLVQXGGSLXLSCXASGXTXXXYAXGWFRQAPGKEXEXVXXIXXXXGXXYYADSVXGRFTISRDNAKXTVYLQMNSLKPEDTAVYYCAADGFDTPCVAGTDWXYDYWGQGTQVTVSS

SEQ ID NO: 306 - protein, 112 aa, Artificial Sequence
  Description of Artificial Sequence Synthetic polypeptide
QVQLVESGGGLVQPGGSLRLSCAASGSIFSINAMGWYRQAPGKQRELVAAITSGGSTNYADSVKGRFTISRDNAKNTVYLQMNSLKPEDTAVYYCNAEYDYWGQGTQVTVSS

SEQ ID NO: 307 - protein, 120 aa, Artificial Sequence
  Description of Artificial Sequence Synthetic polypeptide
XVQLVESGGGLVQPGGSLRLSCAASGXIXSIXAXGWXRQAPGKQREXVAXIXXGGXTNYADSVKGRFTISRDNAKNTVYLQMNSLKPEDTAVYYCXAPMIYYGGRXXDYWGQGTQVTVSS

SEQ ID NO: 308 - protein, 113 aa, Artificial Sequence
  Description of Artificial Sequence Synthetic polypeptide
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCAADFGSWGQGTQVTVSS

SEQ ID NO: 309 - protein, 124 aa, Artificial Sequence
  Description of Artificial Sequence Synthetic polypeptide
EVQLVESGGGLVQAGGSLRLSCAXSGFTFDDYXIXWFRQAPGKEREGXXCIXXSXXXTYYXDSVKGRFTISSDNAKNTVYLQMNSLNPEDTXVYYCAADEPLIVWNCNGDFGSWGQGTQVTVSS

SEQ ID NO: 310 - protein, 122 aa, Artificial Sequence
  Description of Artificial Sequence Synthetic polypeptide
XVQLVESGGGLVQAGGSLRLSCAASGFSFNDYAIGWFRQXPGKEREGVSCLSSSGXAHYADSVKGRFTIXXDNAKNTVYLQMNSLKPEDTAVYXCAXXPFRCGNWRTXMGSWGQGTQVTVSS

SEQ ID NO: 311 - protein, 122 aa, Artificial Sequence
  Description of Artificial Sequence Synthetic polypeptide
XVQLVESGGGLVQAGGSLRLSCXXSGFTFDDYXIGWFRQAPGKXREGXSCISXSGXAHYXDSVKGRFTISSDNAKNTVYLQMNSLKPEDTXXYYCAXXPFRCGNWRTXMGSWGQGTQVTVSS

SEQ ID NO: 312 - protein, 123 aa, Artificial Sequence
  Description of Artificial Sequence Synthetic polypeptide
EVQLVESGGGLVQAGGSLRLSCEASTSMFSIRAATWYRQAPGKQRELVANIDSEGTTGYSDSVKGRFTISRDNTKKTVYLQMNSLKPEDTAVYSCNAVVTYNMLVYDSVYDSWGQGTQVTVSS

SEQ ID NO: 313 - protein, 123 aa, Artificial Sequence
  Description of Artificial Sequence Synthetic polypeptide
XVQLVESGGGLVQAGGSLRLSCXASTSXFSIRAAXWYRQAPGKQRELVAXIDSEGTTGYXDSVKGRFTISRDNXKXTVYLQMNSLKPEDTAVYXCNAVVTYNXLVYDSVYDSWGQGTQVTVSS

(19) United States
(12) Patent Application Publication
(10) Pub. No.: US 2015/0158934 A1
(43) Pub. Date: Jun. 11, 2015

(54) BROADLY NEUTRALIZING VHH AGAINST HIV-1

(71) Applicants: Laura Ellen Fleet McCoy, London (GB); Lucy Rutten, Utrecht (NL); Nika Mindy Strokappe, Utrecht (NL); Cornelis Theodorus Verrips, Utrecht (NL); Benjamin Lucian John Webb, London (GB); Robert Anthony Weiss, London (GB)

(72) Inventors: Laura Ellen Fleet McCoy, London (GB); Lucy Rutten, Utrecht (NL); Nika Mindy Strokappe, Utrecht (NL); Cornelis Theodorus Verrips, Utrecht (NL); Benjamin Lucian John Webb, London (GB); Robert Anthony Weiss, London (GB)

(73) Assignees: UCL BUSINESS PLC, London (GB); UNIVERSITEIT UTRECHT HOLDING B.V., Utrecht (NL)

(21) Appl. No.: 14/343,430

(22) PCT Filed: Sep. 10, 2012
(86) PCT No.: PCT/NL2012/050636

§ 371 Date: Jan. 8, 2015

(30) Foreign Application Priority Data
Sep. 9, 2011 (EP) 11180823.4

(51) Int. Cl. C07K 16/10

(52) U.S. Cl. CPC C07K 16/1063; C07K 16/1045; C07K 2317/31; C07K 2317/565; C07K 2317/76

(57) ABSTRACT

The invention relates to a novel class of broadly neutralizing anti-HIV antibodies, more specifically to broadly neutralizing heavy chain variable domain antibodies (VHH) and variants and modifications thereof. The invention further relates to methods for producing these antibodies and to the use of the antibodies for diagnostic and therapeutic and/or prophylactic treatment of individuals that are infected with HIV, or are at risk of becoming infected.

### SEQUENCE LISTING

The instant application contains a Sequence Listing which has been submitted electronically in ASCII format and is hereby incorporated by reference in its entirety. Said ASCII copy, created on Jul. 10, 2014, is named 362346.00011_SL.txt and is 382,783 bytes in size.

### TECHNICAL FIELD OF THE INVENTION

The present invention relates to the field of virology. More specifically, the invention relates to single heavy chain variable domain antibodies which bind and neutralize a broad range of HIV subtypes. These antibodies are useful for the treatment of individuals infected with HIV and as prophylactic agent, for example as microbicide and as trapping antibodies in apheresis equipment.

### BACKGROUND

Neutralizing antibodies against the human immunodeficiency virus type 1 (HIV-1) are powerful tools not only for understanding the virus structure (Labrijn et al. (2003) J Virol 77(19): 10557-10565; Zhou et al. (2007) Nature 445(7129): 732-737; Liu et al. (2008) Nature 455(7209): 109-113) and the mechanism of cellular entry (Herrera et al. (2005) Virology 338(1): 154-172; Moore et al. (2006) J Viral 80(5): 2515-2528), but also for passive immunization (Trkola et al. (2005) Nat Med 11(6): 615-622; Trkola et al. (2008) J Viral 82(3): 1591-1599; Huber et al. (2008) J Virol 82(8): 3834-3842). Many monoclonal antibodies specific for HIV-1 envelope proteins, gp120 and gp41, have been isolated [http://www.hiv.lanl.gov/content/immunology] both from immunised animals and infected individuals. However, only a few of these are broad neutralizing. These rare antibodies, including b12, 2G12, 2F5, 4E10 and X5 (Burton et al. (2005) Proc Natl. Acad Sci USA 102(42): 14943-14948; Stamatatos et al. (2009) Nat Med 15(8): 866-870) have all been derived from HIV-1 subtype B infected patients and, beside 4E10, display limited activity against the globally most prevalent subtype C HIV-1 (Binley et al. (2004) J Virol 78(23): 13232-13252; Gray et al. (2006) PLoS Med 3(7): e255; Peeters M. (2001) Transfus Clin Biol 8(3): 222-225; Xu et al. (2001) J Hum Virol 4(2): 55-61). Recently new promising broadly neutralizing antibodies have been described, which were all selected from patients that had been infected for a long period of time, notably PG16, PG9 ((Pejchal et al. (2010) Proc Natl Acad Sci USA 107(25): 11483-11488; Walker et al. (2009) Science 326(5950): 285-289); VRC01-03 (Wu et al. (2010) Science 329:856-61) and the engineered variant NIH45-46 (Diskin et al. (2011) Science 334(6060): 1289-93) and although many recognize the CD4 binding site (CD4bs) relative small differences in the areas of interaction resulted in quite different neutralization potencies. Isolation and characterisation of further broadly neutralizing antibodies, with specific attention to non-subtype B viruses, may aid the design and development of immunogens capable of inducing a protective antibody immune response in a vaccine setting. Additionally, such antibodies might be developed as specific entry inhibitors for inclusion in HIV-1 microbicides (Chen and Dimitrov (2009) Curr Opin HIV AIDS 4(2): 112-117).

Llamas, likewise other Camelidae, possess conventional antibodies and heavy chain antibodies. The latter are devoid of light chains (Hamers-Casterman et al. (1993) Nature 363: 446-448) and the variable domain of the heavy chain antibodies (VHH) is therefore solely responsible for antigen recognition. The specificity and affinity of VHH are comparable to IgGs even though the size of a VHH is only approximately 15 kDa. On average, VHH have longer complementarity determining region 3 (CDR3) (Dumoulin et al. (2002) Protein Sci 11(3): 500-515; Muyldermans S. (2001) J Biotechnol 74(4): 277-302; Vu et al. (1997) Mol Immunol 34: 1121-1131) a feature that might facilitate binding into deeper cavities on the antigen surface and that is thought to be important for potent neutralization of HIV-1 via the envelope spike (Pejchal et al. (2010) Proc Natl Acad Sci USA 107(25): 11483-11488; Pancera et al. (2010) J Virol 84(16): 8098-8110). Grooves and cavities play a crucial role in multiple biological activities as these often form the specific interaction site between two molecules (Muyldermans S. (2001) J Biotechnol 74(4): 277-302). Moreover the small size of VHH may be an important property to inhibit transmission of HIV in viral synapsis (Anderson et al. (2010) AIDS 24(2): 163-187). The high stability (Dumoulin et al. (2002) Protein Sci 11(3): 500-515; Dolk et al. (2005) Appl Environ Microbiol 71(1): 442-450; Perez et al. (2001) Biochemistry 40(1): 74-83; Saerens et al. (2008) J Mol Biol 377(2): 478-488; van der Linden et al. (1999) Biochim Biophys Acta 1431(1): 37-46) and the often excellent expression yield of VHH in microbial fermentations (Hultberg et al. (2007) BMC Biotechnol 7: 58; Thomassen et al. (2005) J Biotechnol 118(3): 270-277) make VHH realistic candidates for the development of microbicides to protect against HIV infections.

Thus far, antibodies that neutralize different isolates of HIV have been isolated from individuals that were infected with HIV (Wu et al. 2010. Science 329: 856-861). We recently showed for the first time that neutralizing VHH can be raised in llamas immunized with gp120 of HIV-1_{CN54} (Forsman et al. (2008) J Virol 82(24): 12069-12081). Although the selected VHH exhibited neutralizing effects against HIV-1 primary isolates of subtype B and to a lesser extent subtypes C, they did not neutralize HIV-1 subtypes A, AIG and D.

### SUMMARY OF THE INVENTION

In this invention, Llama single heavy chain variable domain antibody fragments (VHH) have been raised in various lamas using prolonged immunization strategies and selection with traditional panning and sCD4 elution but also with a novel direct neutralization method. The thus isolated VHHs are broadly neutralizing and very potent with high binding affinities.

The selections resulted in the isolation of VHH that bind to amino acids of the CD4 binding site of HIV. The isolated VHH do not, or not to any significant degree, bind to amino acids located around this CD4 binding site. As the amino acids around the CD4 binding site can be mutated without destroying the potency of the virus to bind CD4, these amino acids are quite variable. Antibodies that rely on these residues to bind well to a particular HIV isolate will not achieve broad neutralization over a large number of different HIV isolates.

The invention therefore provides a broadly neutralizing anti-HIV single heavy chain variable domain antibody (VHH). A preferred single heavy chain variable domain antibody neutralizes at least 50% of individual viruses of at least 5 different subgroups (subtypes) of viruses. A further preferred single heavy chain variable domain antibody binds to a CD4-binding site on envelope gp120 protein of HIV. A preferred heavy chain variable domain antibody comprises a combination of CDR1, CDR2 and CDR3 amino acid sequences as depicted in table 3 or table 6. A further preferred heavy chain variable domain antibody comprises amino acid sequences as depicted in table 2. A further preferred heavy chain variable domain antibody neutralizes at least 50% of individual viruses of at least 5 different subgroups (subtypes) of viruses, binds to a CD4-binding site on envelope gp120 protein of HIV, and has a CDR2 region as defined by amino acid residues 52-58 (according to the Kabat numbering) that consists of 5 amino acids.

In addition, the invention also provides derivatives of the heavy chain variable domain antibodies, comprising alterations of the indicated amino acid sequence to increase its production efficiency, broadness and/or physical stability, and conservative derivatives of these antibodies. In addition, a de-immunized and/or humanized variant of a heavy chain variable domain antibody according to the invention is also provided.

A preferred heavy chain variable domain antibody according to the invention comprises a CDR2 region as defined by amino acid residues 52-58 that is characterized by a deletion of three amino acids, when compared to the sequences that are encoded by the corresponding germ line genes, and consists of 5 amino acids. The numbering of amino acid residues 52-58 (containing residue 52a) is according to Kabat (Kabat et al. (1991) Sequences of proteins of immunological interest (5th edn.). U. S. Department of Health and Human Services. Bethesda, Md., USA). The numbering according to Kabat has been integrated into a data base for immunoglobulins, developed by IMGT (Lefranc et al., 1999. Nucleic Acids Research, 27, 209-212), which allow insertion of two or three amino acid residues in the CDR2 region, dependent on the germ line sequence. This data base and the numbering therein are presently the standard. The CDR2 as defined herein corresponds to amino acid residues 57-66 according to the IMGT numbering.

The invention further provides an antibody comprising a broadly neutralizing single heavy chain variable domain antibody according to the invention that has been provided with means for prolonging the biological half life of the single heavy chain variable domain, and/or means for eliminating the bound virus or cells carrying HIV envelop proteins on their surface via antibody-dependent cell-mediated cytotoxicity (ADCC) routes and/or complement dependent cytotoxicity (CDC) routes. In one embodiment, a single heavy chain variable domain antibody according to the invention is fused to an immunoglobulin Fc region or functional part thereof, preferably a Fc region or functional part thereof from IgGl, IgG2, IgG3, IgG4), preferably IgG1. Said Fc region or functional part thereof preferably is a human Fc region or a humanized llama Fc region or functional part thereof.

The invention further provides a bi- or multispecific antibody comprising a heavy chain variable domain antibody according to the invention. Said bi- or multispecific antibody preferably comprises at least two non-competing and non-interfering monovalent anti-HIV heavy chain variable domain antibodies according to the invention. Said multispecific antibodies may further comprise a VHH that prolongs the half life time of the multispecific antibodies in the human body, in addition to a VHH according to the invention.

The invention further provides a nucleic acid molecule encoding an antibody according to the invention, and a method for producing an antibody of the invention, the method comprising expressing said nucleic acid molecule in a relevant cell and recovering the thus produced antibody from the cell. Further provided is a host cell comprising said nucleic acid molecule.

The invention further provides an antibody according to the invention for use as a medicament, preferably for prophylactic or therapeutic treatment of an individual infected with HIV. Provided is a microbicide comprising an antibody of the invention and an apheresis device comprising a broadly neutralizing anti-HIV antibody according to the invention. Further provided is an antibody according to the invention, preferably a labeled antibody, for use in diagnostic applications.

The invention further provides a composition, preferably a pharmaceutical composition comprising an antibody of the invention.

The invention additionally provides an antibody that binds to amino acid residues D368 and I371 or V371 (I/V371), often called the CD4-Phe-43 pocket, both within the CD4 binding site of HIV MB gp120 protein, and which interacts with less than 4, more preferable less than 3 amino acids outside of the CD4 binding site, preferably whereby the interactions with amino acids outside of the CD4 binding site are weak interactions.

### BRIEF DESCRIPTION OF THE DRAWINGS

FIG. 1: Dendrogram of the llama 8 (A) and 9 (B) VHH. The VHH were first aligned with use of ClustalX, the dendrogram was made with use of FigTree. The clones highlighted in black were selected for more extensive characterization.

FIG. 2. Defining preferred epitopes for broadly neutralizing VHH.

The left half of the figure shows the binding sites of CD4 and known antibodies to gp120. The right part of the figure shows our findings. A) The gp120 structure is shown as a surface representation. The residues involved in binding of the particular molecules are colored according to the color of the label of the figure. Contact amino acid residues, as defined by Kwong et al. 1998 (Kwong et al. 1998. Nature 393, 648-659), including the residues of gp120 that have lost solvent-accessible surface, but are not in direct contact, are 123, 124, 125, 126, 257, 278, 279, 280, 281, 282, 283, 364, 365, 366, 367, 368, 370, 371, 425, 426, 427, 428, 429, 430, 455, 456, 457, 458, 459, 469, 471, 472, 473, 474, 475, 476 and 477. In the figures with binding sites other than CD4, the black outline shows the location of the CD4 binding site. In the case of VRC01 the dark pink color represents the epitope for the heavy chain and the light pink color for the light chain. The known antibodies that overlap with the CD4bs, also bind considerably outside the CD4bs. In the case of b12 and 17b the single overlapping residue (R419) is shown in purple. L81H9 does not compete with CD4, but it competes with both b12 and 17b. Based on the cross-competition assay, 1B5 also binds in this region. B) The L93E3 model is represented as ribbons. Gp120 is grey. L93E3 is in cyan with the CRD3 in yellow and the CDR2 in red. The two key residues, Y100 and R100d that interact with the CD4-Phe-43 pocket and D368 respectively of gp120 are shown in all atom model. D368 of gp120 is also shown in all atom model and is colored magenta. C) The whole putative L93E3 binding site is colored cyan. Based on the cross competition assay, antibodies of the L8Cj3 family also bind to the L93E3 binding site. D) The putative L81H9 family binding site. This figure is rotated with respect to the figure at the left of it, by 90° clockwise, around the Y-axis. The yellow residues do not alter between gp120 of CA18, for which the affinity of L82B4F, a family member of L81H9, is very low, and another gp120 for which the affinity is high. The grey ones are not analyzed. The orange residues (363, 369 and 413) are different in a residue that has similar properties and the red ones (389 and 442) have larger differences. The purple residues (419 and 388) are the ones that result in a significant reduction in binding when replaced by an alanine in the alanine scan with gp120-ZM96. Residues 388, 389 and 419 are most likely part of the L81H9/L82B4F epitope (the most preferred epitope comprises residues 388 and 419). E) The preferred epitope that was defined is shown. The brown residues are the preferred residues (based on HXB2 numbering: 279, 281, 282, 367, 368, 371, 473, 474, 425, 430, 472, 476). The dark brown residues are the more preferred residues (279, 281, 282, 367, 368, 371, 473, 474) and the arrows indicate the most preferred: 368 and the CD4-Phe-43 pocket. The VHHs bind to this epitope and do not bind to more than four amino acids outside the CD4 binding site, i.e. 123, 124, 125, 126, 257, 278, 279, 280, 281, 282, 283, 364, 365, 366, 367, 368, 370, 371, 425, 426, 427, 428, 429, 430, 455, 456, 457, 458, 459, 469, 471, 472, 473, 474, 475, 476 and 477. Numbering is all according the HXB2 numbering.

FIG. 3: A) Overview of pTT5-hFc, the plasmid used to construct the VHH-hinge-Fc. B) the individual PCRs necessary to fuse the VHH to the hinge and Fc region.

FIG. 4: In-gel-Western detection with anti-HIV-1 VHH.

8% Acrylamide gel with purified HIV-1 derived gp140 protein from gp120 of HIV-1CN54 and HIV-1UG37 and human plasma loaded, incubated with IRDye labeled anti-gp140 VHH (L91F10) and with Coomassie Brilliant Blue. After extensive washing the gels were scanned with the LI-COR Odyssey at 700 and 800 nm. The 700 nm wavelength channel is shown in red and shows most of the marker bands. The 800 nm wavelength channel is shown in green and shows the IR-Dye800CW labeled anti-gp140 VHHs. The Coomassie stained protein bands are visible in the 700 nm channel, but are presented here in grey-scale.

FIG. 5: Construction of VHH-Fc fusions. A) Schematic representation of construction of VHH-Fc fusions. Two PCR products digested with the restriction enzymes BsmBI and NotI were ligated in the mammalian expression vector, which was digested with BsmBI and NotI as well. B) One letter codes of amino acids of the fusion between L8Cj3 with the Fc fragment of human IgG1, consisting of the hinge region and the CH2 and CH3 domains. The hinge region derived from human IgG1 is represented in bold case and is underlined. The sequence of VHH L8Cj3 is N-terminal of the hinge region and the sequence of wild type human IgG1 is C-terminal of the hinge region. L8Cj3-Fc(ADCC+) with enhanced ADCC function has two mutations compared to the wild type sequence, i.e. S239D and I332E, shown in white letters with a black background below the wild type sequence. L8Cj3-Fc(FD), which is deficient in ADCC function, has three mutations compared to the wild type sequence, i.e. E233P, L234V and L235A, in the hinge region shown in bold case below the wild type sequence. FIG. 5B discloses SEQ ID NO: 270. C) Schematic representation of VHH-Fc fusions with the monospecific VHH-Fc fusions at the left and the bispecific VHH-Fc fusion at the right. The table at the right shows all VHH-Fc fusion constructs that have been made and of which proteins have been produced and purified. At the right the four bispecific VHH-Fc fusions that were produced are shown (note that in the mixture both the homodimers and the heterodimers are present).

FIG. 6: Constructed L8Cj3 mutants and a family member, L8i5.

A) Alignment of amino acid sequences of the germ line Vt (SEQ ID NO: 271), L8Cj3 (SEQ ID NO: 207) and L8i5 (SEQ ID NO: 209). Amino acids with a grey background in L8Cj3 differ from the germ line sequence and the grey amino acids in L8i5 differ from L8Cj3. Framework regions are indicated by FR1, FR2, FR3 and FR4. CDR regions are indicated by CDR1, CDR2 and CDR3. Numbers in bold above the germ line sequence and number 12 below the L8i5 sequence indicates the 13 mutations that were made in L8Cj3 by site directed mutagenesis. Most mutations are reversals to the germ line indicated by bold font and underlined in the germ line sequence, except for mutants 8, 9 and 12.

B) L8Cj3 mutants and L8i5 are shown in the left column. Affinities of the L8Cj3 mutants to gp140 of UG037 and CN54 are shown in the middle two columns. 50% binding concentrations are given in μg/ml as K50 (Values below 1 μg/ml are marked in dark grey, between 1 and 10 μg/ml in medium grey, between 10 and 100 μg/ml in light grey). Neutralization of the L8Cj3 mutant of 92UG037 pseudovirus and CH181 virus as IC50 values in μg/ml are shown in the right two columns. Values below 1 μg/ml are marked in dark grey, between 1 and 10 μg/ml in medium grey, between 10 and 50 μg/ml in light grey. >50 means that the strain was not neutralized at the highest concentration tested.

C) The neutralization IC50 values of eight HIV-1 viruses and pseudoviruses in μg/ml of L8Cj3 compared with J3r. Values below 1 μg/ml are marked in dark grey and between 1 and 10 μg/ml are marked in medium grey.

D) Extensions of the CDR2s of L8Cj3 and L93E3. Three types of extensions of the CDR2 were constructed for L8Cj3. Firstly the insertion of the germ line S, secondly the insertion of the germ line SW and thirdly the insertion of the germ line SWS. All insertions abrogated binding to gp140UG037 and gp140CN54 and neutralization of HIV-1 92UG037 pseudovirus and CH181 virus. All values, the KD and IC50 values, are in μg/ml. FIG. 6D discloses SEQ ID NOS 274-278, respectively, in order of appearance.

E) The germ line DGS (or SDG) of the CDR2 were inserted in 3E3mod (containing the mutations V5Q, P14A, E82aN with respect to L93E3). 3E3mod binds to gp140 and neutralizes HIV-1, whereas 3E3mod with the extended CDR2 does not. FIG. 6E discloses SEQ ID NOS 280 and 279-280, respectively, in order of appearance.

F) L8Cj3 and L93E3 and their variants with extended CDR2s are shown in the left column. Affinities of the L8Cj3 mutants to gp140 of UG037 and CN54 are shown in the middle two columns. 50% binding concentrations are given in μg/ml as K50 (Values below 1 μg/ml are marked in dark grey, between 1 and 10 μg/ml in medium grey, between 10 and 100 μg/ml in light grey). Neutralization of the L8Cj3 mutant of 92UG037 pseudovirus and CH181 virus as IC50 values in μg/ml are shown in the right two columns. Values below 1 μg/ml are marked in dark grey, between 1 and 10 μg/ml in medium grey, between 10 and 50 μg/ml in light grey. >50 means that the strain was not neutralized at the highest concentration tested. ND stands for not determined.

FIG. 7: Schematic representation of the envelope spike (PDB IDs: 3DNN for the gp120 trimer, 2X7R for the gp41 hexamer). Gp41 is indicated in grey and on the top right, the three gp120 subunits are colored white, grey and dark grey and are on the bottom and left of the figure. The exact binding site of sCD4 is visible in one of the gp120 subunits and highlighted with a thin black line. The residues of gp41 to which the neutralizing antibody 4E10 binds are highlighted in black (top left) and those that 2F5 binds in white (also top left). The putative binding area of 1B5/1H9/2B4F is indicated with a dotted black oval (Left, in the middle), 1F10 with a grey oval (left bottom), 2E7/11F1F/1E2 with dashed black circle (in the middle) and 5B10D/9B6B/11F1B with a black circle (to the right of the middle).

FIG. 8: Immuno-fluorescent images using myc-tagged J3

Human Monocyte-Derived-Macrophages (MDM) were infected with HIV-1 Bal for 7 days. Stained J3-myc tagged with anti myc 4A6 murine antibody, goat anti-mouse IgG2 was labeled with Alexa 594 was used to visualize HIV, whereas 4C9 anti p17 mouse antibody was visualized using goat anti-mouse IgG2 labeled with Alexa. The merged colours are indicated in the upper right panel.

FIG. 9: Immuno-fluorescent images using myc-tagged 3E3

Human Monocyte-Derived-Macrophages (MDM) were infected with HIV-1 Bal for 7 days. 3E3-myc tagged was stained with anti myc 4A6 murine antibody, goat anti-mouse IgG2 was labeled with Alexa 594 was used to visualize HIV, whereas 4C9 anti p17 mouse antibody was visualized using goat anti-mouse IgG2 labeled with Alexa. The merged colours are indicated in the upper right panel.

FIG. 10: EM-images using myc-tagged J3

Human Monocyte-Derived-Macrophages (MDM) were infected with HIV-1 Bal for 7 days. HIV was detected using VHHJ3-with myc tag, 9E10 anti-myc murine antibody, visualized with rabbit anti-mouse antibody bound to gold-coated protein A. Gold particles are 10 nm gold particles.

FIG. 11: Surface representation of gp120. The darker grey area indicates the L8Cj3 epitope within the CD4 binding site (black outline).

### DETAILED DESCRIPTION OF THE INVENTION

### Definitions

The term “antibody” as used herein, refers to an antigen binding protein comprising at least a heavy chain variable region (Vh) that binds to a target epitope. The term antibody includes monoclonal antibodies comprising immunoglobulin heavy and light chain molecules, single heavy chain variable domain antibodies, and variants and derivatives thereof, including chimeric variants of monoclonal and single heavy chain variable domain antibodies.

The term “VHH”, as used herein, refers to single heavy chain variable domain antibodies devoid of light chains. Preferably a VHH is an antibody of the type that can be found in Camelidae or cartilaginous fish which are naturally devoid of light chains or a synthetic VHH which can be constructed accordingly.

As described herein, the amino acid sequence and structure of a heavy chain variable domain, including a VHH, can be considered—without however being limited thereto—to be comprised of four framework regions or ‘FR’, which are referred to in the art and herein as ‘Framework region 1’ or ‘FR1’; as ‘Framework region 2’ or ‘FR2’; as ‘Framework region 3’ or ‘FR3’; and as ‘Framework region 4’ or ‘FR4’, respectively; which framework regions are interrupted by three complementary determining regions or ‘CDR′s’, which are referred to in the art as ‘Complementarity Determining Region 1’ or ‘CDR1’; as ‘Complementarity Determining Region 2’ or ‘CDR2’; and as ‘Complementarity Determining Region 3’ or ‘CDR3’, respectively.

The amino acid residues of heavy chain variable regions, including VHH, are numbered according to the general numbering of Kabat (Kabat, et al. (1991) Sequences of Proteins of Immunological Interest, 5th edition. Public Health Service, NIH, Bethesda, Md.). For the purpose of this patent application, amino acid residues 26-33 of VHH are defined as CDR1, amino acid residues 52-58 of VHH are defined as CDR2, and amino acid residues 95-103 of VHH are defined as CDR3, with the amino acid residue numbering according to the Kabat numbering.

As also further described herein, the total number of amino acid residues in a VHH is typically in the region of 110-120, is preferably 112-115, and is most preferably 113.

The term ‘binding’ as used herein in the context of binding between an antibody, preferably a VHH, and an epitope of HIV as a target, refers to the process of a non-covalent interaction between molecules. Preferably, said binding is specific. The terms ‘specific’ or ‘specificity’ or grammatical variations thereof refer to the number of different types of antigens or their epitopes to which a particular antibody such as a VHH can bind. The specificity of an antibody can be determined based on affinity. A specific antibody preferably has a binding affinity Kd for its epitope of less than 10⁻⁷ M, preferably less than 10⁻⁸ M.

The term HIV refers to the human retroviral human immunodeficiency virus. The term HIV refers to HIV-2 and HIV-1 retroviruses, preferably HIV-1 retroviruses including groups O, N, P and M of HIV-1 and subgroups (also termed subtypes or clades) thereof.

The term epitope or antigenic determinant refers to a part of an antigen that is recognized by an antibody. The term epitope includes linear epitopes and conformational epitopes. A conformational epitope is based on 3-D surface features and shape and/or tertiary structure of the antigen.

The term neutralizing antibody refers to an antibody that, when bound to an epitope, interferes with at least one of the steps leading to the release of the viral genome into a host cell.

The term broadly neutralizing as used herein refers to neutralization of HIV viruses of different groups or subgroups (subtypes/clades). A broadly neutralizing antibody preferably neutralizes at least 65% of individual viruses that belong to at least 3 different groups or subgroups of HIV. Preferred subgroups include subgroups A, A/C, A/E, A/C/D, A/G, B, B/C, C, C/D, D, F, G, H, J and K2 of the M-group of HIV-1.

The term affinity refers to the strength of a binding reaction between a binding domain of an antibody and an epitope. It is the sum of the attractive and repulsive forces operating between the binding domain and the epitope. The term affinity, as used herein, refers to the dissociation constant, Kd.

The term gp140_{CN54} (subtype B′/C) refers to gp140 envelope glycoprotein from the HIV-1 group M CRF07-B/C-clade isolate 97CN54 (AF286226).

The term gp140_{UG37} (subtype A) refers to gp140 envelope glycoprotein from the HIV-1 group M A-clade isolate 92UG037 (AB253429).

The term CD4 binding site (CD4bs) refers to a region on the gp120 envelope protein of HIV that interacts with the CD4 receptor and which is conserved in different groups or subgroups of HIV. The binding surface of conventional neutralizing antibodies often includes amino acid residues of the CD4bs, but includes interaction with amino acid residues outside of this region.

The terms “strong interaction” and “strong binding” refers to the presence of salt bridges and cation-pi interactions between amino acid residues, as is known to the skilled person.

The terms “weak interaction” and “weak binding” refers to the presence of hydrogen bonds and non-bonded/hydrophobic interactions, as is known to the skilled person.

The term microbicide refers to products that contain active components that block transfer of viruses of other harmful living entities into the human body, in particular via the vagina and/or rectum.

The term acquired immunodeficiency syndrome (AIDS) refers a severe immunological disorder caused by HIV, resulting in a defect in cell-mediated immune response that is manifested by increased susceptibility to opportunistic infections and to certain rare cancers such as Kaposi's sarcoma.

The term “broadly neutralizing VHH”, as is used herein, refers to a VHH that neutralizes at least 50%, more preferred at least 65%, more preferred at least 75%, preferably at least 85%, even more preferred more than 90% and most preferred more than 92% of individual HIV viruses that belong to different groups or subgroups (subtypes/clades), such as the HIV species given in Table 1. Said individual HIV viruses preferably represent a broad range of viruses VHH (Seaman et al. 2010. J Virol 84: 1439-1452), which are used world wide to assess the broadness of HIV reagents.

### Broadly Neutralizing VHH

The present invention relates to a specific class of antibodies, namely single heavy chain variable domain antibody antibodies (VHH) which are capable of broadly neutralizing HIV. The term “broadly neutralizing” has been introduced in the literature by several groups (Corti D et al. 2010. PLoS One; 5(1): e8805; Scheid et al. 2011. Science 333: 1633-7; Walker et al. 2011. Nature 477: 466-70; Walker et al. 2009. Science 326: 285-9; Wu et al. 2010. Science 329: 856-61; Pejchal et al. 2010. Proc Natl Acad Sci USA. 107: 11483-8; Walker et al. 2010. PLoS Pathog; 6(8): e1001028; Hesse11 and Haigwood 2012. Curr HIV/AIDS Rep 9: 64-72; Burton and Weiss 2010. Science 29: 770-3). A panel of 109 HIV-1 Env pseudoviruses, representing a broad range of viruses, has been suggested to assess the breadth of the neutralizing capacity of antibodies, including VHH (Seaman et al. 2010. J Virol 84: 1439-1452). Reference panels for subgroup B (clade B) and subgroup C (clade C) viruses have been described by Li et al. 2005. J Virol 79: 10108-25 and Li et al. 2006. J Virol 80: 11776-90, respectively.

The heavy chain variable domain antibody antibodies were isolated from llamas that were immunized with mixtures of two different antigens, gp140_{CN54} (subtype B′/C) and gp140_{UG37} (subtype A) to promote development of broadly reactive VHH. The llamas were injected for a total of 7 times. The final boost injection was at 113 days after the first injection, where the final boost injection is normally provided at 35 days after the first injection. Immune phage display libraries were generated from these animals at 43 and 122 days after the first injection. These libraries were screened directly for neutralization of HIV. This resulted in the isolation of a large number of VHHs that revealed neutralizing activities against a panel of primary HIV-1 including A, B and C subtypes. Without wishing to be bound by theory, it is believed that the prolonged immunization period of the llamas, combined with panning and/or the direct screening for neutralization of HIV resulted in the isolation of this large number of broadly neutralizing VHHs. These VHHs serve as a powerful tool to improve treatment of individuals that are infected with HIV. The invention therefore provides a broadly neutralizing anti-HIV heavy chain variable domain antibody (VHH) that neutralizes at least 65%, more preferred at least 75%, more preferred at least 85%, more preferred at least 90%, more preferred at least 92%, of individual HIV viruses that belong to different groups or subgroups. A preferred broadly neutralizing antibody that is isolated according to the methods herein described is capable of neutralizing at least 50% of individual viruses, more preferred at least 70%, more preferred at least 75%, more preferred at least 80%, more preferred at least 85%, more preferred at least 90%, more preferred at least 95% of individual viruses that belong to at least 3 different groups or subgroups of HIV, more preferred at least 5 different groups or subgroups, more preferred at least 7 different groups or subgroups, more preferred at least 8 different groups or subgroups, more preferred at least 9 different groups or subgroups, most preferred at least 10 different groups or subgroups of HIV. A most preferred broadly neutralizing antibody according to the invention is capable of neutralizing at least 50% of individual viruses of more than 5 different groups or subgroups (subtypes/clades). This means that at least 50% of the tested viruses of each of the at least 5 clades, preferably at least 2, more preferred at least 3 different viruses of each subgroup (clade), are neutralized by the antibody. Said at least 2 different viruses of each subgroup are preferably selected from a tier 1 and a tier 2 strain, more preferred a tier 1 and a tier 3 strain, The term tier refers the classification of HIV-1 viral strains according to their pattern of sensitivity to antibody-mediated neutralization when tested against a panel of genetically diverse HIV-1-positive plasma pools. The four tier groups are defined as: those having very high (tier 1A), above-average (tier 1B), moderate (tier 2), or low (tier 3) sensitivity to antibody-mediated neutralization (Seaman, et al. 2010. J. Virol. 84(3): 1439-1452).

An anti-HIV heavy chain variable domain antibody according to the invention preferably has a binding affinity of at most 10⁻⁷ M, more preferred at most 10⁻⁸ M, more preferred at most 10⁻⁹ M, more preferred at most 10⁻¹⁰ M, more preferred at most 10⁻¹¹ M, most preferred at most 10⁻¹²M.

A VHH according to the invention preferably comprises CDR1, CDR2 and CDR3 amino acid sequences as depicted in table 3 or table 6, or a derivative, for example a conservative derivative, thereof. Preferred derivatives comprise alterations of the amino acid sequence of the VHH to increase its efficiency, broadness and physical stability as, for example, indicated in table 6. A further preferred VHH antibody comprises any of the variable domain amino acid sequences as depicted in table 2 or a derivative, for example a conservative derivative, thereof. A further preferred VHH antibody comprises any of the variable domain amino acid sequences as depicted in table 11 or a derivative, for example a conservative derivative, thereof. The term “conservative derivative” as used herein denotes the replacement of an amino acid residue by another, biologically similar residue. Examples of conservative derivatives include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like.

The isolated VHH can be grouped into 7 families termed L81H9, L91B5, L94D4, L93E3, L91F10, L8Cj3, and L92E7, according to the germ line V- and J-region of which the isolated VHH have been derived by selection and maturation (see table 3). In addition, individual VHH with broad neutralizing capabilities were isolated.

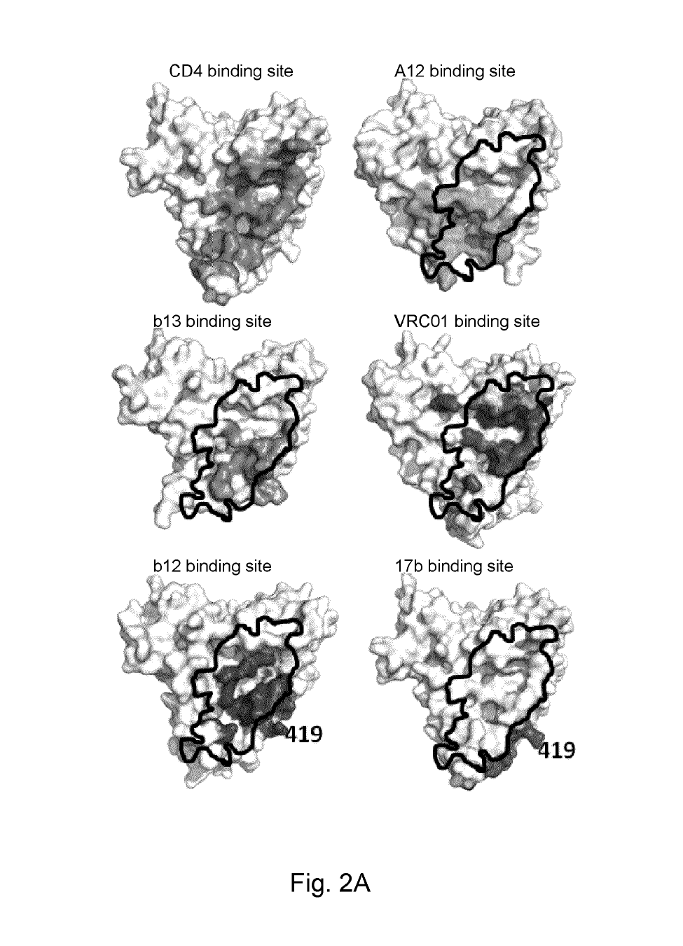

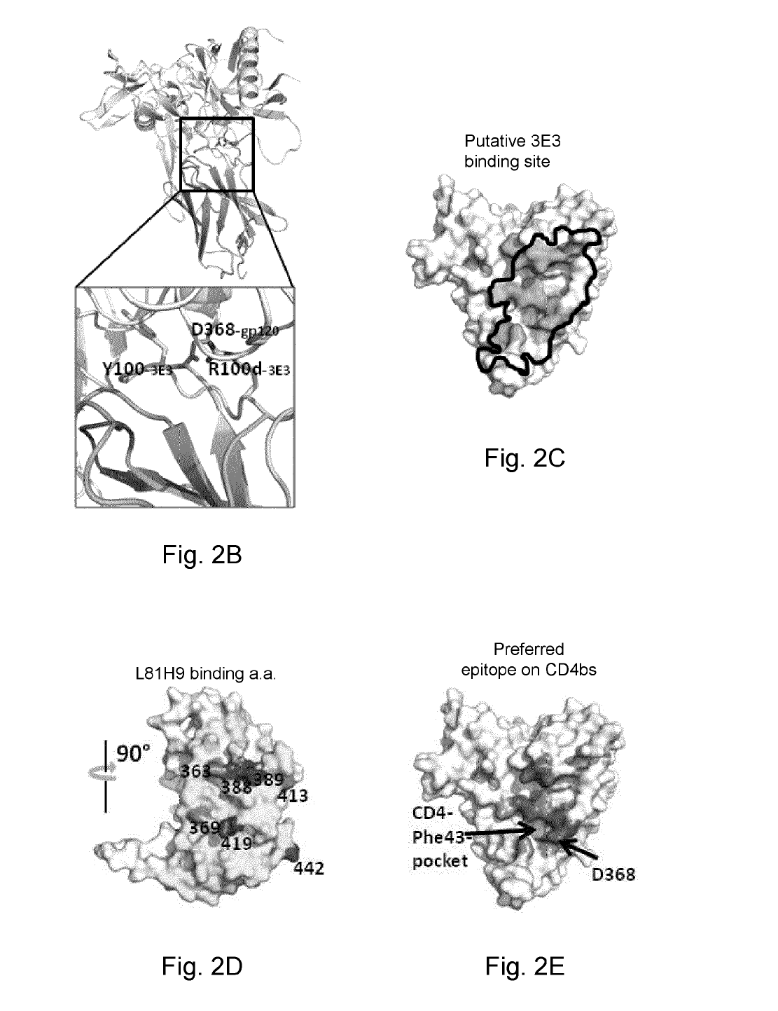

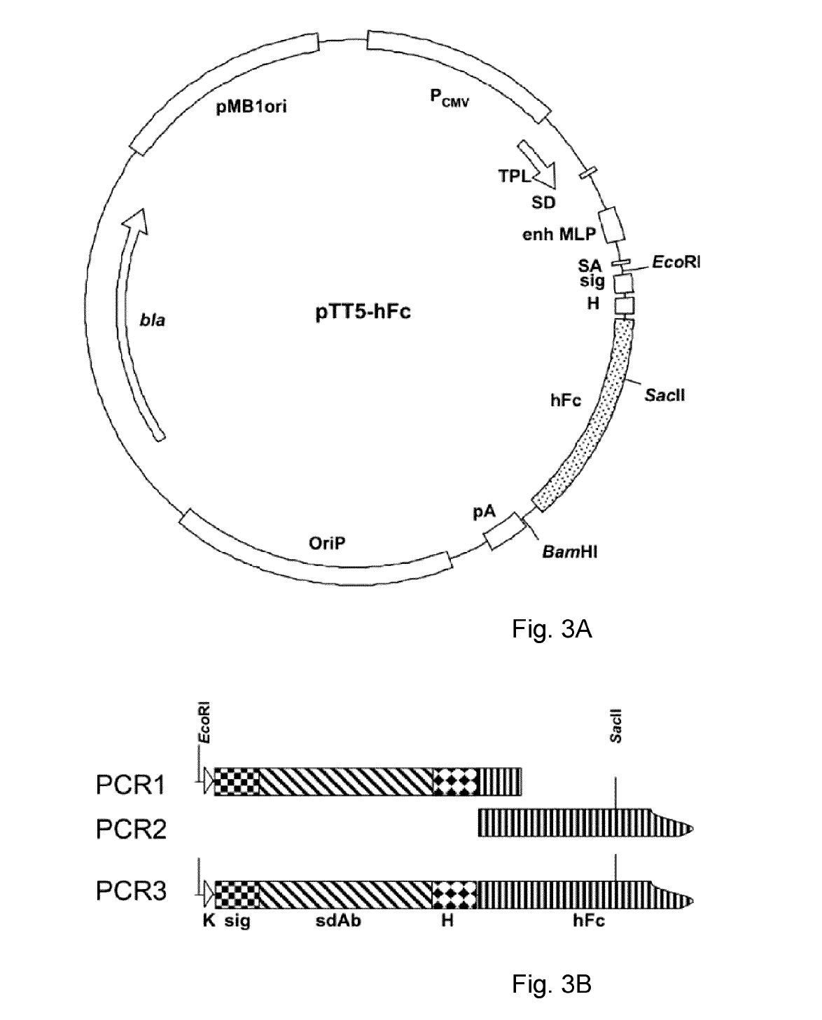

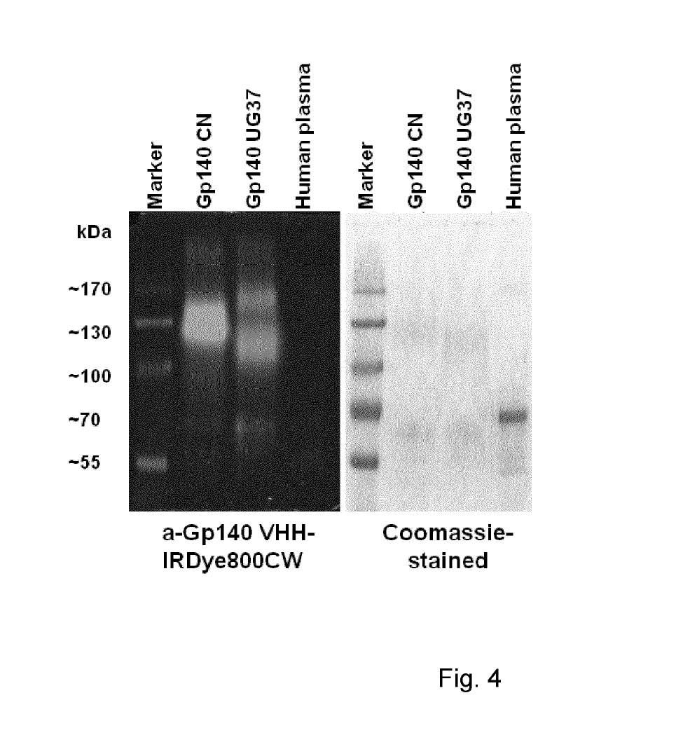

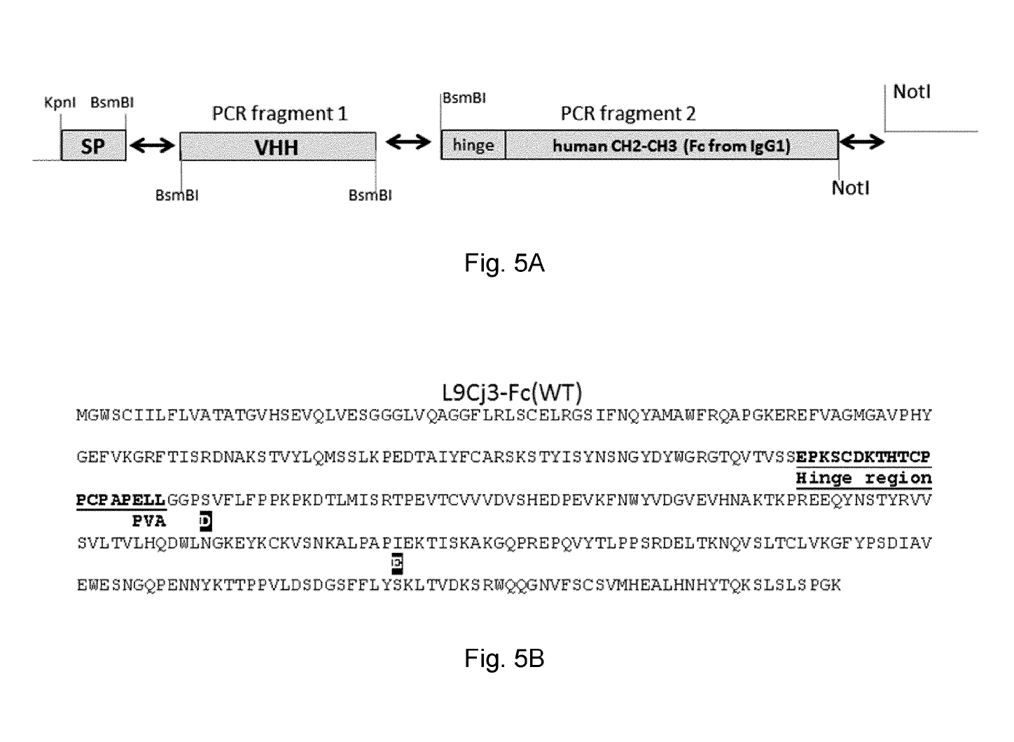

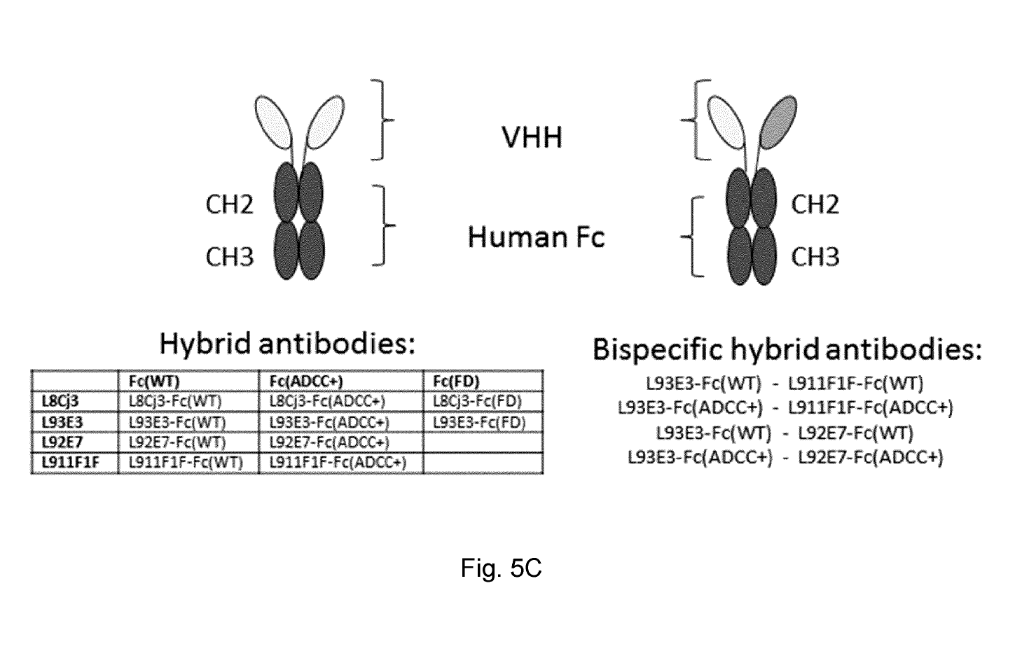

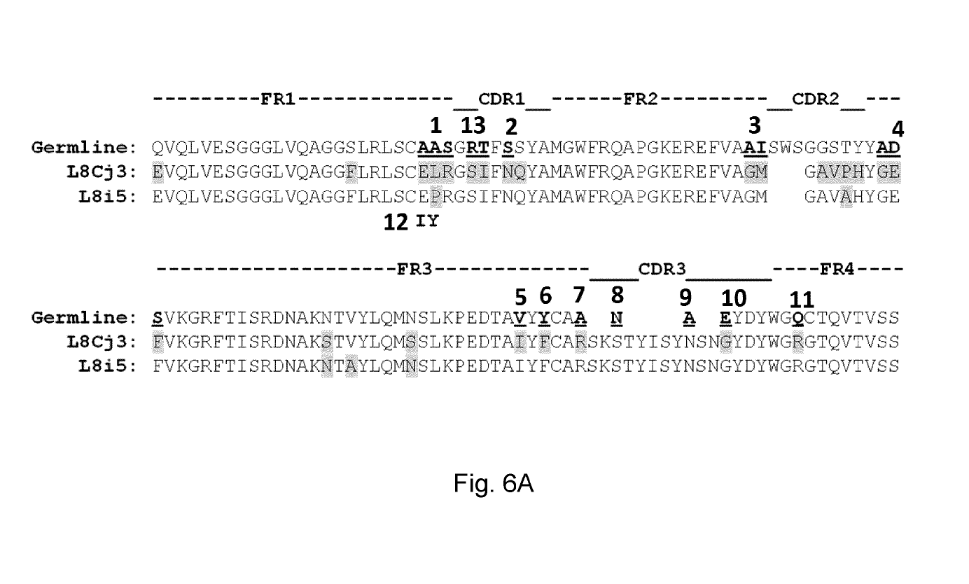

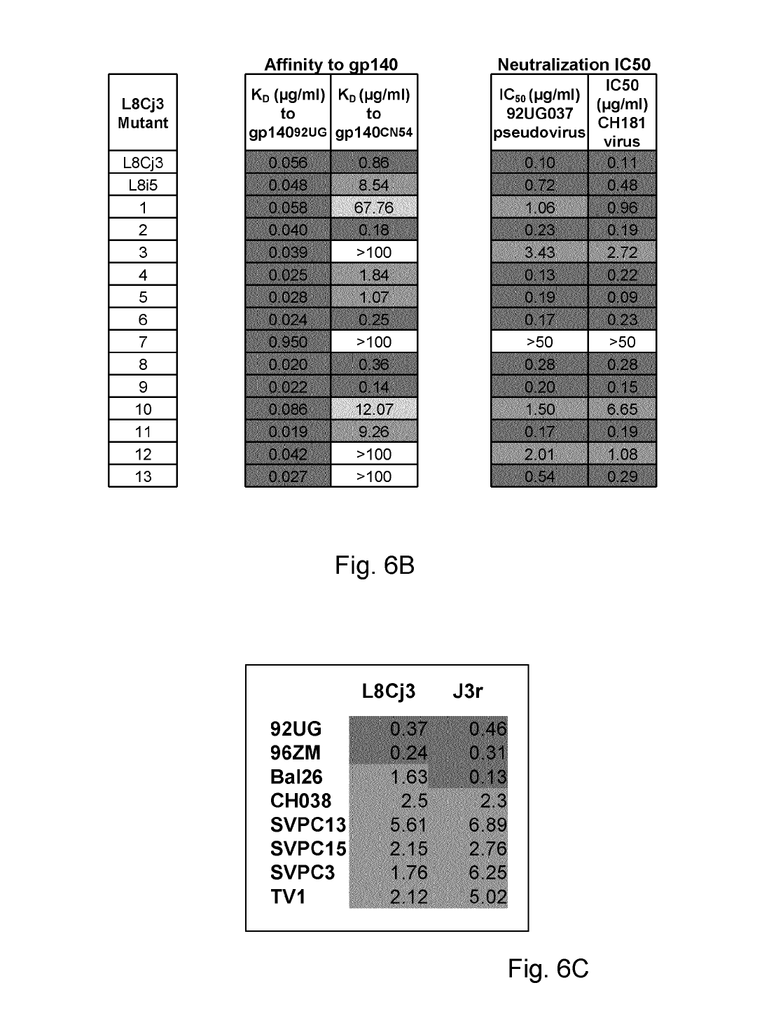

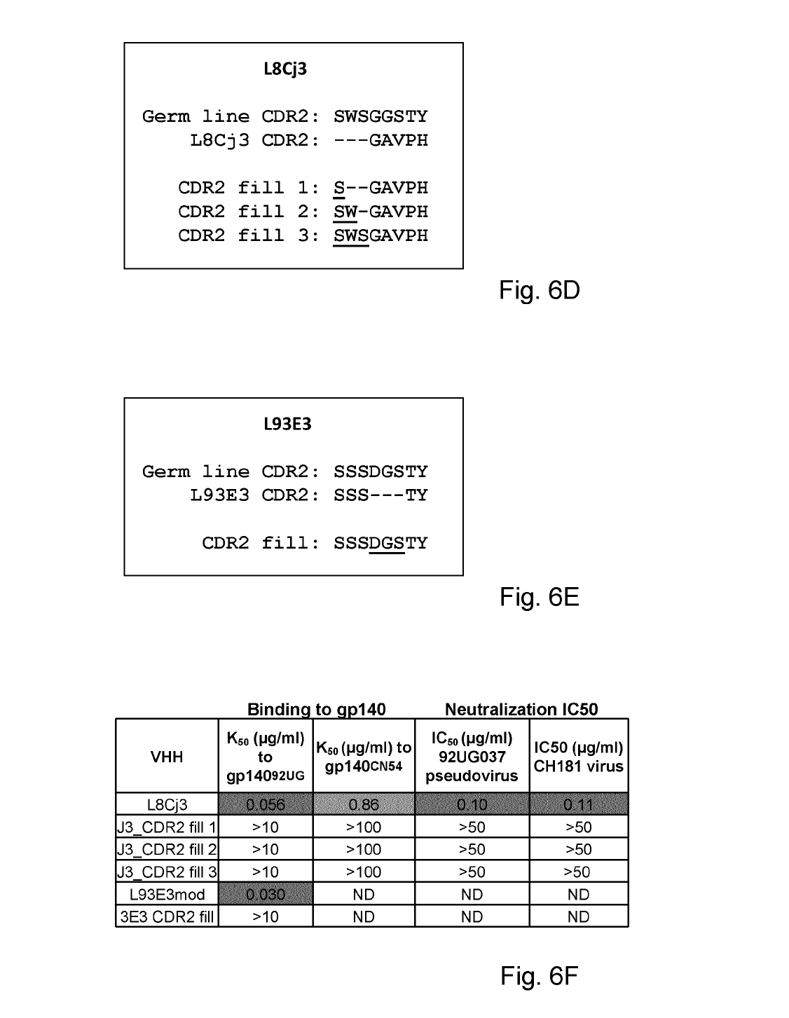

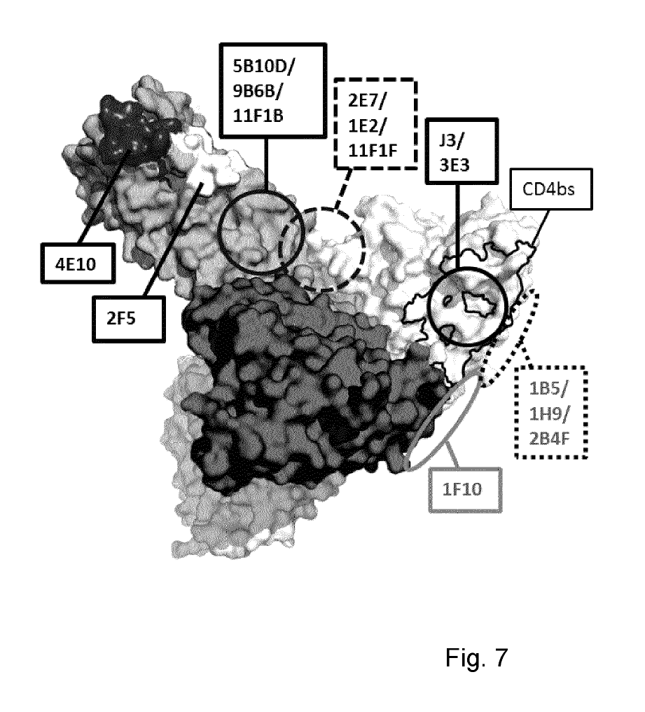

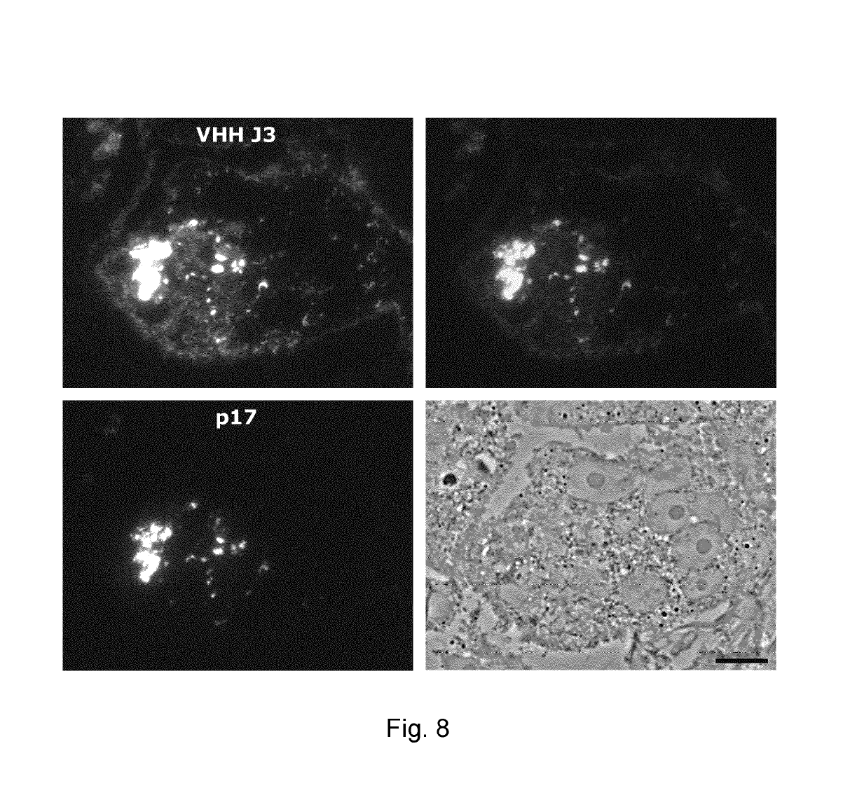

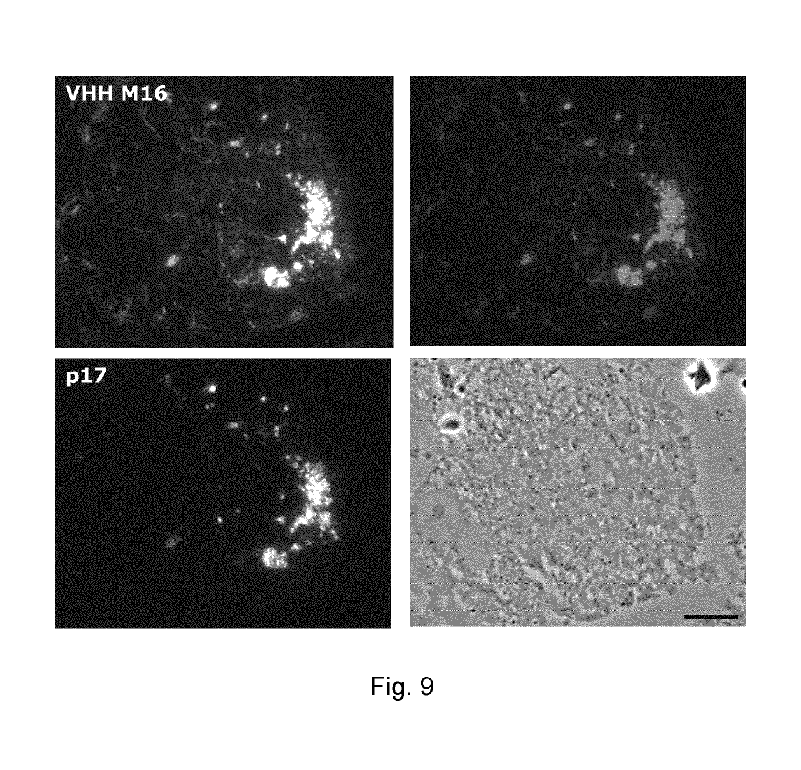

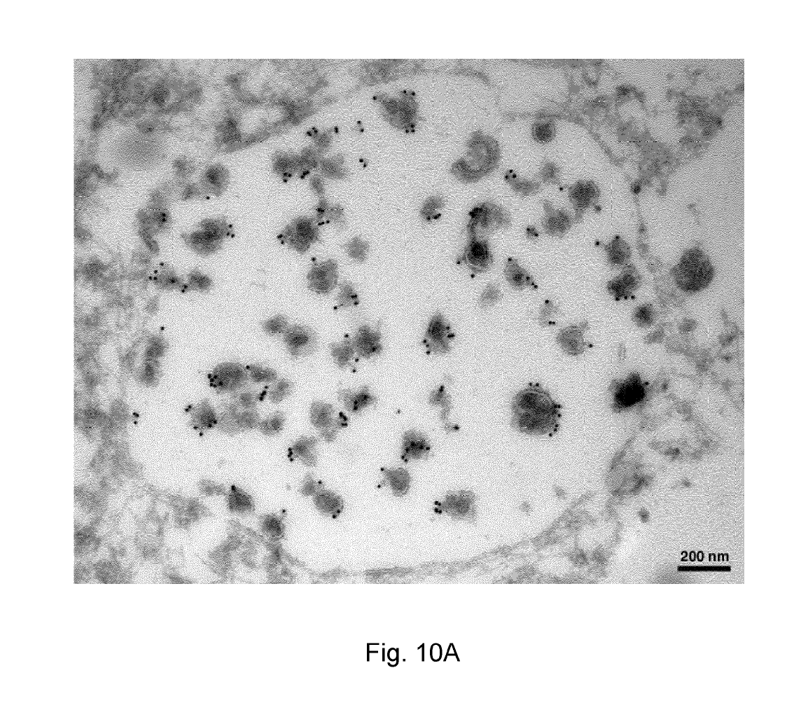

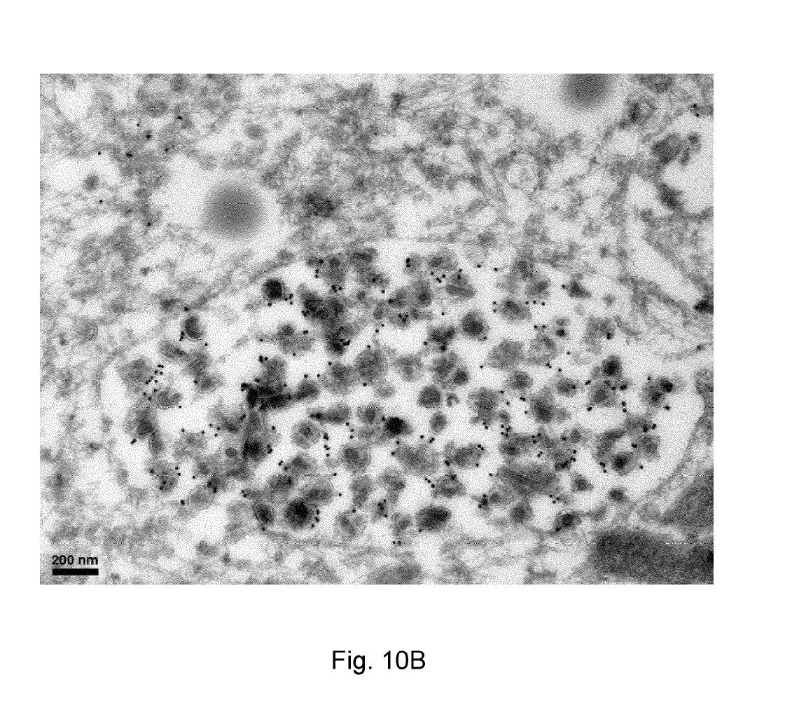

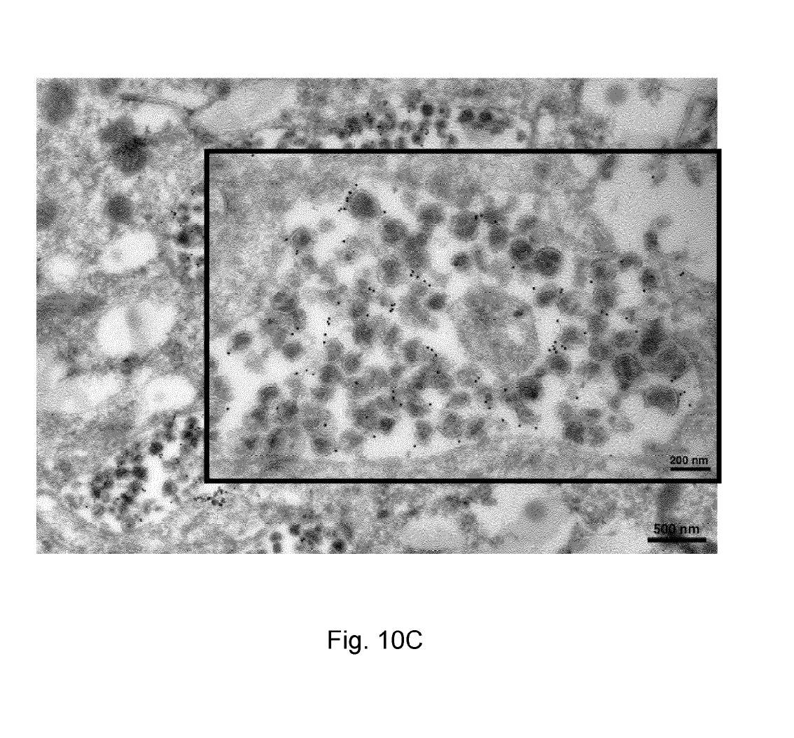

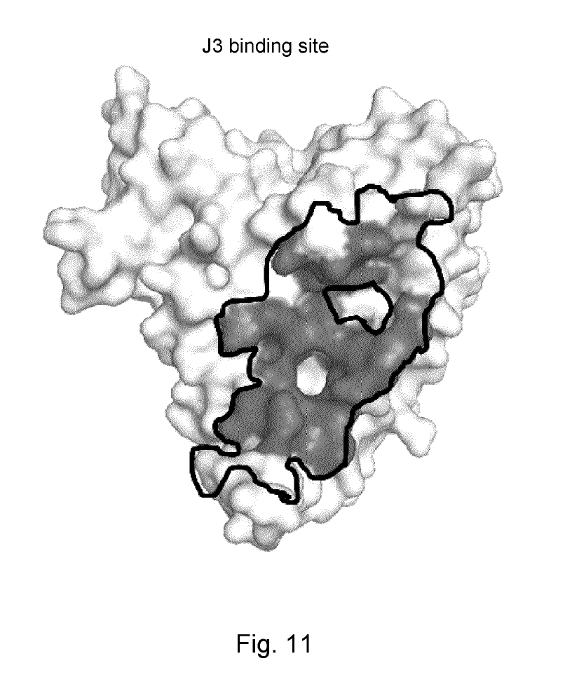

Two of the isolated families, L93E3 and L8Cj3, bind nearly exclusively to the CD4bs on gp120. The CD4bs is characterized by a low frequency of mutation and is essential for binding to CD4 and therefore for the survival of the HIV. The selected VHH interact with a conserved amino acid, D368, and a pocket comprising I/V371 within the CD4bs (see FIG. 2 and Table 9). The nearly perfect interaction between these VHH and these conserved residues result in high binding constants of the selected VHH to gp120/gp140. Interestingly, the interaction site is limited to the CD4bs. No strong interactions were identified between VHH that belong to the L93E3 and L8Cj3 families and amino acids outside the CD4bs (see FIG. 11), in contrast to conventional antibodies that are larger than VHH and also make contacts outside the CD4bs. The fact that the interaction site of VHH belonging to the L93E3 and L8Cj3 families is limited to the conserved CD4bs might at least in part explain the exceptionally broad neutralization capacity of these VHH, being more than 90% of different HIV-1 retrovirus species representing more than 10 different subgroups of HIV for members of the L8Cj3 family, and more than 85% of different HIV-1 retrovirus species representing more than 10 different subgroups of HIV for members of the L93E3 family.

Another remarkable finding was that these 2 families of VHH, which originated from different llamas and were derived from different V-genes, are both characterized by a deletion in CDR2, reducing it size from 8 to 5 amino acids. A shortened CDR2 in J3 is required for binding to its epitope and for neutralization, as reinsertion of one, two or all three germ line residues in the CDR2 abrogated binding to its epitope and neutralization of HIV (see example 8.3). Inspection of the interaction between these VHH and their cognate antigen showed that this deletion was essential to get precise binding of the VHH on D368 and I/V371 (CD4-Phe-43 pocket) and that this deletion reduces the sizes of the surfaces of these VHH that interact with the antigen. The invention therefore provides an anti-HIV heavy chain variable domain antibody, wherein the CDR2 region as defined by amino acid residues 52-58 is characterized by a deletion of 3 amino acids from the germ line encoded sequence and consists of 5 amino acids instead of 8 amino acids that are present in the germ line.

Using the same libraries and similar selection procedures, we also selected at least 2 families of VHH, L81H9 and L91B5, which do not recognize the CD4bs. This is surprising as soluble CD4 was used for elution. Even more surprising was the finding that these VHH are broadly neutralizing (>50%) although less potent than VHH that recognize the CD4bs. However their breadth makes them excellent partners for the VHH recognizing the CD4bs to construct very broad neutralizing, very potent bi-functional VHH. The probability that the HIV virus develops escape mutants against these bi-functional VHH is extremely small.

The remaining three families, L94D4, L91F10 and L92E7 are broadly neutralizing and do not compete with L93E3/L8Cj3 for binding to HIV gp120. It was confirmed that the families 1F10 and 4D4 and 2E7 do not to compete with the CD4bs.

VLP1_A14 and VLP1_b9, which are inter-related family members, and VLP3_b21 (see Table 15) were also found to neutralize at least 70% of individual viruses of at least 5 different subgroups (see Table 14). The CDR2 region of all three VHH, VLP1_A14, VLP1_b9, and VLP3_b21, comprises 7 amino acid residues. VLP1_A14 and VLP1_b9 have a deletion of 1 amino acid in the CDR2 region, as compared to the germ line sequences from which these VHH were derived. VLP1_A14, VLP1_b9, and VLP3_b21 bind to the CD4-binding site.

### De-Immunization and Humanization

A heavy chain variable domain antibody is small, does not or hardly aggregate and has a short half life when administered, for example, to humans. Therefore, VHH hardly induce an immune response after administration to humans. However, de-immunization and/or humanization may be required for use of the antibodies of the invention in pharmaceutical compositions. De-immunization is a preferred approach to reduce the immunogenicity of antibodies according to the invention. It involves the identification of linear T-cell epitopes in the antibody of interest, using bioinformatics, and their subsequent replacement by site-directed mutagenesis to non-immunogenic sequences or, preferably human sequences. Methods for de-immunization are known in the art, for example from WO098/52976, which is herein incorporated by reference.

A further preferred approach to circumvent immunogenicity of antibodies according to the invention when applied to humans involves humanization. Various recombinant DNA-based approaches have been established that are aimed at increasing the content of amino acid residues in antibodies that also occur at the same of similar position in human antibodies while retaining the specificity and affinity of the parental non-human antibody. Most preferred are amino acid residues that occur in antibodies as they are encoded by genomic germ line sequences. Humanization may include the construction of VHH-human chimeric antibodies, in which the VHH binding regions are covalently attached, for example by amino acid bonds, to one or more human constant (C) regions.

Further preferred methods for humanizing antibodies according to the invention include grafting of CDRs (Queen, C. et al. (1989) Proc. Natl. Acad. Sci. U.S.A. 86: 10029; Carter, P. et al. (1992) Proc. Natl. Acad. Sci. U.S.A. 89: 4285); resurfacing (Padlan, E. A., et. al., (1991). Mol. Immunol., 28: 489), superhumanization (Tan, P., D. A., et. al., (2002) J. Immol., 169: 1119), human string content optimization (Lazar, G. A., et. al., (2007). Mol. Immunol., 44: 1986) and humaneering (Almagro, J. C., et. al., (2008). Frontiers in Bioscience 13: 1619). These methods rely on analyses of the antibody structure and sequence comparison of the non-human and human antibodies in order to evaluate the impact of the humanization process into immunogenicity of the final product.

### Methods for Isolation and Selection of Broadly Neutralizing Antibodies

The invention further provides a method for obtaining broadly neutralizing antibodies. Said broadly neutralizing antibodies can be obtained from animals including rodents such as rats and mice, rabbits, birds, guinea pigs, pigs, goats and sheep. A preferred broadly neutralizing antibody comprises a VHH. A VHH may be derived from any immunoglobulin naturally devoid of light chains, such that the antigen-binding capacity and specificity is located exclusively in the heavy chain variable domain. Said heavy chain variable domains are preferably obtained from camelids (as described in WO 94/4678), especially Lamas (for example Lama glama, Lama vicugna (Vicugna vicugna) or Lama pacos (Vicugna pacos), or from Camelus (for example Camelus dromedaries or Camelus bactrianus). In another embodiment, said VHH is obtained from a cartilaginous fish. In a further preferred embodiment, said animal is a transgenic animal such as a transgenic mouse that is capable of expressing heavy chain antibodies such as, for example, a transgenic mouse as described in WO 02/085945 and in WO 04/04979.

In a preferred method for obtaining broadly neutralizing antibodies such as VHH, an animal is provided with a HIV particle, preferably an inactivated particle, or an immunogenic part thereof. A preferred part of a HIV particle comprises the envelope comprising the glycoproteins (gp) gp120 and gp41. A preferred part of a HIV particle comprises the glycoprotein precursor gp160, or a modified form thereof, gp140, in which the transmembrane domains from gp41 have been deleted. A further preferred part of a HIV particle is a part of gp120 that interacts with CD4, a receptor of HIV on cells.

Said HIV particle or immunogenic part thereof is preferably derived from HIV-2 or HIV-1, more preferred from group O, N, P or M of HIV-1, most preferred of a M-subtype or clade selected from A, A/C, A/E, A/C/D, A/G, B, B/C, C, C/D, D, F, G, H, J and K2. It is further preferred that the HIV particle or immunogenic part thereof comprises a mixture of particles or parts thereof from different groups and/or subgroups, such as from 2 groups and/or subgroups, from 3 groups and/or subgroups, from 4 groups and/or subgroups, or from 5 or more groups and/or subgroups. Said HIV particle or immunogenic part thereof is preferably derived from M-subgroups A and B, A and C, B and C, or A, B and C.

A preferred HIV particle or immunogenic part thereof comprises gp120_{IIIB} (subgroup B), gp140_{CN54} (subgroup B/C), gp140_{UG37} (subtype A), and/or gp120_{YU2} (subgroup B) and/or a modified variant thereof, gp120Ds2, which comprises additional cysteines at amino acid positions 109 and 428 of gp120, which form a cysteine-bridge to lock the protein in a preferred conformation. Said HIV particle or immunogenic part is preferably obtained from the AIDS Research and Reference Reagent Program (ARRRP), Division of AIDS, NIAID, NIH (USA).

Said HIV particle or immunogenic part thereof may be provided to an animal by any means known to the skilled person. A preferred mode of administration to provide an animal with said HIV particle or immunogenic part thereof is injection, preferably intramuscular injection. The amount that is provided to an animal depends on the size of the animal, as is well known to the skilled person. A preferred amount of a HIV particle or immunogenic part thereof that is administered to a larger animal is between 1 and 500 micrograms of each particle or immunogenic part thereof, more preferred between 2 and 250 micrograms. A most preferred amount for an animal such as a llama is about 100 microgram of each particle or immunogenic part thereof.

Said HIV particle or immunogenic part thereof is preferably provided as an immunogenic composition, in which HIV particle or immunogenic part thereof is admixed with an adjuvant and/or a carrier or other excipient known in the art. Suitable adjuvants include, but are not limited to, aluminium and calcium salts such as aluminium phosphate, aluminium hydroxide, aluminium potassium sulphate (alum) and calcium phosphate, organic adjuvants such as Squalene, lipopolysaccharide or another toll-like receptor stimulating agent, oil-based adjuvants such as complete and incomplete Freund's adjuvant, RIBI Adjuvant System consisting of monophosphoryl lipid A, synthetic trehalose dicorynomycolate, and cell wall skeleton, (Ribi Immunochem Research, Inc., USA)), TiterMax®, consisting of a block copolymer CRL-8941 (Hunter's Titermax; USA)) and Stimune (Specol®). A most preferred adjuvant is Stimune adjuvant, which can be obtained from CEDI Diagnostics, Lelystad, The Netherlands.

It is preferred that the provision of a HIV particle or immunogenic part thereof to an animal is repeated at least once, preferably at least twice, more preferred at least three times, such as three times, four times, five times, six times or more than six times. It is further preferred that the primary provision and the one or more secondary provisions (boosts) are provided to an animal at intervals of at least one week. A preferred mode of administration of a HIV particle or immunogenic part thereof to an animal comprises a primary provision, preferably an injection, at day 0, and a secondary provision, preferably injection, at day 7. In a most preferred embodiment, the secondary provision is repeated at least four times at regular or irregular intervals until a final boost is provided at 100 days or more after the primary provision. Without being bound by theory, the prolonged exposure of an animal to a HIV particle or immunogenic part thereof and/or the late timing of the final boost may help inducing a relative large amount of broadly neutralizing antibodies in the animal. An example of a preferred provision scheme comprises a first provision at day 0, and a boost provisions at day 7, day 14, day 21, day 28, day 35 and day 113. However, a deviation from this scheme is possible when a final boost is provided at 100 days or more after the first provision of a HIV particle or immunogenic part thereof.

The immunogenic composition that is used for the primary and secondary provisions may comprise the same or, preferably, different HIV particles or immunogenic parts thereof, preferably representing different clades and/or the same of a different adjuvant. In addition, the modes of administration may differ, for example the primary provision being an intramuscular injection and the secondary and further provisions being subcutaneous injections. The primary and secondary provisions preferably comprise the same or similar HIV particles or immunogenic parts thereof, the same or a similar adjuvant, and the same or a similar mode of administration.

After the final boost provision, the immunized animal is tested for the presence of HIV-binding and neutralizing antibodies. A preferred method includes the collection of blood of an immunized animal for the isolation of peripheral blood lymphocytes (PBLs). A cDNA library is generated from the PBLs using specific primers that result in amplification of gene fragments that encode antibody binding domains such as conventional and heavy-chain immunoglobulin gene fragments. This cDNA library is cloned in a vector that allows the display of a protein in connection with the genetic information that encodes the protein, such as ribosomal display and phage display (Groves et al. 2006. Journal of Immunological Methods 313: 129-139). In phage display, a library of antibody binding domains is expressed on the surface of filamentous bacteriophage particles. From these libraries, phages are selected by binding to an antigen of interest. This selection is preferably repeated at least once. The genetic information that encodes this antibody binding domain is subsequently expressed as a soluble antibody fragment from infected bacteria. The affinity of binding of a selected antibody binding domain can subsequently be improved by mutation (Winter, G., et. al. (1994). Annu. Rev. Immunol. 12: 433). The process mimics immune selection, and antibodies with many different binding specificities have been isolated using this approach (Hoogenboom, H. R., et. al. (2005). Nat. Biotechnol., 23, 1105),

In a preferred method for obtaining broadly neutralizing antibodies, the selection of an antibody or antibody binding domain using a display technique comprises a HIV virus neutralization assay as a first selection assay. Without being bound by theory, the selection of an antibody or antibody binding domain by affinity selection may result in a bias for antibodies or antibody binding domains that bind with high affinity to the HIV particle or immunogenic part thereof, but do not neutralize the virus. The introduction of a neutralization assay as a first selection assay prevents this bias, resulting in the selection of high amounts of neutralizing antibodies. A most preferred method of the invention therefore combines a prolonged exposure of an animal to a HIV particle or immunogenic part thereof by late timing of a final boost and the introduction of a neutralization assay as a first selection assay.

Neutralization of HIV is assessed by determining the loss of infectivity through reaction of the virus with an antibody or antibody binding domain. Virus and antibody or antibody binding domain are mixed under appropriate conditions and then provided to an indicator cell culture that is sensitive to HIV. The loss of infectivity is brought about by interference by the bound antibody or antibody binding domain with any one of the steps leading to the release of the viral components into the host cells. An antibody or antibody binding domain is preferably assayed for neutralization as a soluble antibody fragment. For efficiently assaying a large number of antibodies or antibody binding domains in a neutralization assay, a high throughout cloning step is preferably provided for the generation of soluble antibody fragment from infected bacteria. The high throughout cloning step preferably includes picking of individual clones with a robot, growing of clones in multiwell plates, for example 96 or 384 well plates, and harvesting of antibodies or antibody binding domains from the supernatant or a periplasmic extract by freeze thawing and subsequent filtration, for example through a 0.2 uM PDVF membrane. Neutralization is preferably measured using 200 50% tissue culture infective doses of virus in the TZM-bl cell-based assay developed by Wei et al. (2002, Antimicrobial Agents and Chemotherapy 46:1896-1905) with Bright-Glo luciferase reagent (Promega).

### Antibodies Comprising a Broadly Neutralizing VHH

The invention further provides an antibody, preferably a bispecific or multispecific antibody, comprising a broadly neutralizing single heavy chain variable domain that binds HIV. Said antibody preferably comprises means for prolonging the biological half life of the single heavy chain variable domain, for example after administration of the single heavy chain variable domain to an individual. Said antibody also preferably comprises means for eliminating the bound virus or cells carrying HIV envelop proteins on their surface via antibody-dependent cell-mediated cytotoxicity (ADCC) routes and/or complement dependent cytotoxicity (CDC) routes.

In a preferred embodiment, said antibody comprises an immunoglobulin Fc region or functional part thereof of an immunoglobulin heavy chain. The Fc region or functional part thereof is preferably derived from IgG1, IgG2, IgG3, IgG4, IgM, IgD, IgA or IgE. It is further preferred that the Fc region or part thereof is a human Fc region or part thereof or a camelid Fc region or part thereof, for example a llama Fc region or part thereof. Said camelid Fc region or part thereof preferably is humanized. The single heavy chain variable domain is preferably connected to an Fc region or functional part thereof via a hinge region. A preferred hinge region is the hinge region of a camelid or human immunoglobulin heavy chain molecules from IgG1, IgG2, IgG3, IgG4, IgM, IgD, IgA or IgE, most preferred from IgG1. A hinge region of a camelid immunoglobulin heavy chain molecule preferably is humanized.

A preferred part of an Fc region is the region comprising the C2 domain, the C3 domain, or the C2 and C3 domains of IgGs, preferably IgG1 or IgG3, most preferably C2 and C3 domains of human IgG1.

A further preferred antibody is a bi- or multivalent antibody comprising a broadly neutralizing single heavy chain variable domain according to the invention. Said bi- or multivalent antibody preferably is a bispecific or multispecific antibody comprising two or more different single heavy chain variable domains that recognize different epitopes on the surface of HIV particles. A bi- or multispecific antibody preferably comprises two or more single heavy chain variable domains that bind HIV, of which at least one is a broadly neutralizing single heavy chain variable domain. Said two or more anti-HIV single heavy chain variable domains are preferably 2 non-competing and non-interfering monovalent anti-HIV VHH. Table 7 provides non-limiting examples of combinations of different non-overlapping anti-HIV VHH. However, a VHH of the invention may be combined with other, preferably non-competing and non-interfering anti-HIV VHH.

A preferred bi- or multispecific antibody according to the invention comprises a VHH that binds to the CD4 binding site, for example selected from L9Cj3 and L93E3, and at least one VHH that is selected from one of the non-competing families L81H9, L91B5, L94D4, L91F10 and L92E7. A further preferred bi- or multispecific antibody comprises a VHH that binds to the CD4 binding site, for example selected from J3 and 3E3, and at least one VHH that binds to the CCR5 binding site, such as, for example, L1719A12, L1720C1, L1720E4 and L2320F9.

Said two or more single heavy chain variable domains are preferably fused to the Fc region or part thereof, preferably comprising the C2 and C3 domains of IgGs, preferably IgG1 or IgG3, most preferably human C2 and C3 domains. The constant region that is fused to the single heavy chain variable domains preferably comprises a dimerization or multimerization motif. Alternatively, a bi- or multispecific antibody may be generated by chemical cross-linking or by a heterologous dimerization or multimerization domain comprising, for example, a leucine zipper or jun-fos interaction domain (Pack and Plückthun, (1992) Biochemistry 31, 1579-1584; de Kruif and Logtenberg, (1996) JBC 271: 7630-7634).

A further preferred bi- or multispecific antibody is a bihead or a multihead VHH, for example a trihead VHH, as described in EP1002861, which is herein incorporated by reference. The bihead or multihead antibodies preferably comprise a linking group which provides conformational flexibility so that each of the single heavy chain variable domains can interact with its epitope. A preferred linker group is a linker polypeptide comprising from 1 to about 50 amino acid residues, preferably from 10 to about 30 amino acid residues, most preferred about 20 amino acid residues such as 15 amino acid residues, 16 amino acid residues, 17 amino acid residues, 18 amino acid residues, 19 amino acid residues, 20 amino acid residues, 21 amino acid residues, 22 amino acid residues, 23 amino acid residues, or 24 amino acid residues. Some preferred examples of such amino acid sequences include gly-ser linkers, for example of the type (gly x ser y) z, such as (for example (gly 4 ser) 3 (SEQ ID NO: 281) or (gly 3 ser 2) 3 (SEQ ID NO: 282), as described in WO 99/42077, which is hereby incorporated by reference, and the GS30, GS15, GS9 and GS7 linkers described in, for example WO 06/040153 and WO 06/122825, both of which are hereby incorporated by reference, as well as hinge-like regions, such as the hinge regions of naturally occurring heavy chain antibodies or similar sequences (such as described in WO 94/04678; which is hereby incorporated by reference). A preferred bihead or multihead comprises at least two non-competing and non-interfering monospecific anti-HIV VHH.

A bihead or a multihead is preferably provided with means to extend the half life of the antibody after administration to a human individual. For example, a preferred bihead or multihead comprises at least one anti-HIV VHH according to the invention and a VHH that interacts with an abundant antigen on the surface of cells, preferably such that it does not result in internalization of the bi-head or multihead. For this, VHH may be used that are directed against, for example, contents of extracellular matrix such as fibronectin and laminin. A further preferred bihead or multihead comprises an IgG1 interacting VHH or a VHH that recognizes human serum albumin (HSA). In addition, a bihead or multihead according to the invention is preferably coupled to an immunoglobulin Fc region, or functional part thereof such as a C2 domain and/or C3 domain of preferably IgG1, to activate intrinsic immunological mechanisms.

### Methods to Produce Broadly Neutralizing Antibodies Comprising a VHH

An antibody according to the invention, for example a single heavy chain variable domain or an antibody comprising a single heavy chain variable domain, may be produced using antibody producing prokaryotic cells or eukaryotic cells, preferably mammalian cells such as CHO cells or HEK cells, or fungi, most preferably filamentous fungi or yeasts such as Saccharomyces cerevisiae or Pichia pastoris, or mouse ascites. An advantage of a eukaryotic production system is that folding of the protein results in proteins that are more suitable for treating a human individual. Moreover, eukaryotic cells often carry out desirable post translational modifications that resemble posttranslational modifications that occur in mammalian cells.

Production of VHH in filamentous fungi is preferably performed as described by Joosten et al. (2005). J Biotechnol 120:347-359, which is included herein by reference. A preferred method for producing VHHs in Saccharomyces cerevisiae is according to the method as described by v. d. Laar et al, (2007), Biotech Bioeng 96, 483-494; or Frenken et al. (2000). J Biotechnol 78:11-21, which are included herein by reference. Another preferred method of VHH production is by expression in Pichia pastoris as described by Rahbarizadeh et al. (2006) J Mol Immunol 43:426-435, which is included herein by reference.

A further preferred method for production of therapeutic VHH comprises mammalian cells such as a fibroblast cell, a Chinese hamster ovary cell, a mouse cell, a kidney cell, a retina cell, or a derivative of any of these cells. A most preferred cell is a human cell such as, but not limited to, Hek293 and PER.C6. A further preferred cell line is a cell line in which alpha-(1,6)-fucosyltransferase has been inactivated, for example the •FUT8 CHO cell line, as described in Yamane-Ohnuki et al 2004, Biotechnol. Bioeng 87, 614-622. It was found that antibodies that are produced in •FUT8 cells enhance the ADCC route.

An antibody according to the invention is preferably produced by the provision of a nucleic acid encoding said antibody to a cell of interest. Therefore, the invention further provides a nucleic acid encoding an antibody according to the invention. Said nucleic acid, preferably DNA, is preferably produced by recombinant technologies, including the use of polymerases, restriction enzymes, and ligases, from the single heavy chain variable domains that were isolated from the immunized animal, as is known to a skilled person. Alternatively, said nucleic acid is provided by artificial gene synthesis, for example by synthesis of partially or completely overlapping oligonucleotides, or by a combination of organic chemistry and recombinant technologies, as is known to the skilled person. Said nucleic acid is preferably codon-optimised to enhance expression of the antibody in the selected cell or cell line. Further optimization preferably includes removal of cryptic splice sites, removal of cryptic polyA tails and/or removal of sequences that lead to unfavourable folding of the mRNA. The presence of an intron flanked by splice sites may encourage export from the nucleus. In addition, the nucleic acid preferably encodes a protein export signal for secretion of the antibody out of the cell into the periplasm of prokaryotes or into the growth medium, allowing efficient purification of the antibody.

The invention further provides a vector comprising a nucleic acid encoding an antibody according to the invention. Said vector preferably additionally comprises means for high expression levels such as strong promoters, for example of viral origin (e.g., human cytomegalovirus) or promoters derived from genes that are highly expressed in a cell such as a mammalian cell (Running Deer and Allison (2004) Biotechnol Prog 20: 880-889; U.S. Pat. No. 5,888,809). The vectors preferably comprise selection systems such as, for example, expression of glutamine synthetase or expression of dihydrofolate reductase for amplification of the vector in a suitable recipient cell, as is known to the skilled person.

The invention further provides a method for producing an antibody, the method comprising expressing a nucleic acid according to the invention in a prokaryotic or eukaryotic cell and recovering the thus produced antibody from the cell. The nucleic acid, preferably a vector comprising the nucleic acid, is preferably provided to a cell by transfection or electroporation. The nucleic acid is either transiently, or, preferably, stably provided to the cell. Methods for transfection or electroporation of cells with nucleic acid are known to the skilled person. A cell that expresses high amounts of the antibody is selected. This cell is grown, for example in roller bottles, in fed-batch culture or continuous perfusion culture. An intermediate production scale is provided by an expression system comprising disposable bags and which uses wave-induced agitation (Birch and Racher (2006). Advanced Drug Delivery Reviews 58: 671-685). Methods for purification of antibodies are known in the art and are generally based on chromatography, such as protein A affinity and ion exchange, to remove contaminants. In addition to contaminants, it may also be necessary to remove undesirable derivatives of the product itself such as degradation products and aggregates. Suitable purification process steps are provided in Berthold and Walter (1994). Biologicals 22: 135-150.

The invention additionally provides a host cell comprising a nucleic acid or vector according to the invention. Said host cell may be grown or stored for future production of an antibody according to the invention.

### Use of an Antibody Comprising a VHH.

The invention further relates to a product or composition containing or comprising at least one broadly neutralizing anti-HIV antibody as described herein. Therefore, the invention provides an antibody according to the invention for use as a medicament. The antibodies of the invention are preferably used for prophylactic administration or therapeutic administration in both humans and other animals that are infected with HIV or closely related viruses like SHIV. Thus, the antibodies according to the invention are administered to high-risk individuals in order to lessen the likelihood of developing AIDS in these individuals or to lessen the severity of the disease, or administered to subjects already evidencing active HIV infection.

The administration of an antibody of the invention is preferably provided in an effective amount. An effective amount of an antibody of the invention is a dosage large enough to produce the desired effect in which the symptoms of the HIV infection are ameliorated or the likelihood of infection is decreased. A therapeutically effective amount preferably does not cause adverse side effects, such as hyperviscosity syndrome, pulmonary edema, congestive heart failure, and the like. Generally, a therapeutically effective amount varies with the subject's age, condition, and sex, as well as the extent of the disease in the subject and can be determined by one of skill in the art. The dosage may be adjusted by the individual physician or veterinarian in the event of any complication. A therapeutically effective amount may vary from about 0.01 mg/kg to about 500 mg/kg, preferably from about 0.1 mg/kg to about 200 mg/kg, most preferably from about 0.2 mg/kg to about 20 mg/kg, in one or more dose administrations daily, for one or several days. Preferred is administration of the antibody for 2 to 5 or more consecutive days in order to avoid “rebound” of virus replication from occurring.

The antibodies of the invention can be administered by injection or by gradual infusion over time. The administration of the antibodies preferably is parenteral such as, for example, intravenous, intraperitoneal, or intramuscular. Preparations for parenteral administration include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

An antibody according to the invention may be linked (chemically or otherwise) to one or more groups or moieties that extend the half-life (such as PEG or BSA or HSA), so as to provide a derivative of an amino acid sequence of the invention with an increased effective half life in vivo after administration to a patient. General methods for coupling of antibodies are described in WO 2011/049449, which is hereby incorporated by reference. Methods for pegylating an antibody are known in the art, for example from U.S. Pat. No. 7,981,398, which is hereby incorporated by reference.

A preferred therapeutic use of a broadly neutralizing anti-HIV antibody of the invention is as a trapping antibody in apheresis equipment, offering the opportunity to reduce high viral titers and/or high levels of infected cells of the immune system. The term apheresis refers to treatment methods whose therapeutic effects are based on the extra-corporeal elimination of pathogenic particles, pathogenic proteins, protein-bound pathogenic substances, free pathogenic substances or pathogenic cells of the blood. Preferred methods are selective whole blood apheresis methods, in which HIV retrovirus proteins are specifically adsorbed directly from the non-pretreated blood without plasma separation. A further preferred apheresis method is termed cytapheresis, in which cells that are infected with HIV such as leukocytes, erythrocytes, thrombocytes, granulocytes or even stem cells are removed from the blood. A broadly neutralizing anti-HIV antibody according to the invention is preferably coupled to a solid carrier capable of being contacted with the blood or plasma flow in an apheresis device. The invention therefore also provides an apheresis device comprising a broadly neutralizing anti-HIV antibody according to the invention that is coupled to a solid carrier capable of being contacted with blood or plasma flow. Such carriers, methods or devices have been described for example in WO97/48483 and in U.S. Pat. Nos. 5,476,715, 6,036,614, 5,817,528 and 6,551,266, which are all herein incorporated by reference.

A preferred prophylactic use of a broadly neutralizing anti-HIV antibody according to the invention is provided by a microbicide. The term microbicide refers to a product that is used topically, preferably vaginally or rectally, to prevent infection. A microbicide offers the potential for women to protect themselves and their sexual partners from HIV. For other anti-infective use, they may also be applied to the skin, mucous membranes, and orally. Preferred microbicides are inexpensive, affordable, stable at ambient temperature, preferably at temperatures above 35° C., more preferably at temperatures above 40° C., compatible and active after mixture with cosmetically acceptable formulations, non-toxic and non-damaging to vulvar, vaginal, cervical, penile or other epithelium. A microbicide preferably further comprises a base or carrier, such as a foam, cream, wash, gel, suppository, ovule, lotion, ointment, film, tablet, foaming tablet, tampon, vaginal spray, aerosol, or other base or carrier as would be apparent to a skilled person. Said microbicide is preferably coupled to a support, for example a vaginal ring, for providing sustained protection against HIV, as described, for example in Wahren et al. 2010. Journal of Translational Medicine 8:72,

The invention further provides a composition comprising an antibody according to the invention. Said composition preferably is a pharmaceutical composition. A pharmaceutical composition preferably comprises a pharmaceutically acceptable carrier. A carrier, as used herein, means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts buffers, stabilizers, solubilizers, and other materials which are well known in the art.

An anti-HIV antibody of the invention may be labeled by a variety of means for use in diagnostic applications. There are many different labels and methods of labeling known to those of ordinary skill in the art. Examples of the types of labels which can be used in the present invention include enzymes, radioisotopes, fluorescent compounds, colloidal metals, chemiluminescent compounds, infra red dyes, and bioluminescent compounds. For in vivo diagnosis, radioisotopes may be bound to immunoglobulin either directly or indirectly by using an intermediate functional group. Intermediate functional groups which are often used to bind radioisotopes which exist as metallic ions are the bifunctional chelating agents such as diethylenetriaminepentacetic acid (DTPA) and ethylenediaminetetra-acetic acid (EDTA) and similar molecules. Typical examples of metallic ions which can be bound to the anti-HIV antibodies of the invention are .sup.111In, .sup.97Ru, .sup.67Ga, .sup.68Ga.sup.72As, .sup.89Zr and .sup.201Tl. The antibodies of the invention can also be labeled with an infrared dye or with a paramagnetic isotope for purposes of in vivo diagnosis as in, for example, magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include 157Gd, .sup.55Mn, .sup.162Dy, .sup.52Cr and .sup.56Fe. Those of ordinary skill in the art will know of other suitable labels for binding to the antibodies of the invention, or will be able to ascertain such, using routine experimentation. Furthermore, the binding of these labels to the antibodies of the invention can be done using standard techniques common to those of ordinary skill in the art.

Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

The antibodies of the invention may further be used in vitro, for example, in immunoassays in which they can be utilized for detection of antigens in liquid phase or bound to a solid phase carrier. The antibodies in these immunoassays can be detectably labeled in various ways. Examples of types of immunoassays which can utilize the antibodies of the invention are competitive and non-competitive immunoassays. The assays either comprise a direct or an indirect format and include radioimmunoassay (RIA) and the sandwich assay. HIV present in biological fluids and tissues can be detected by the antibodies of the invention. A sample can be a liquid such as urine, saliva, cerebrospinal fluid, blood serum or the like; a solid or semi-solid such as tissues, feces, or the like; or alternatively, a solid tissue such as those commonly used in histological diagnosis.

### Epitopes

The broadly neutralizing anti-HIV heavy chain variable domain antibodies bind to different epitopes on the surface of the envelope gp120 protein of HIV. A preferred epitope resides in the CD4bs of HIV, and which is bound by antibodies of families L93E3 and L8Cj3. Said epitope contains at least the amino acid residues D368 and I/V371, and preferably contains D279, A281, K282, G367, D368, I/V371, G473 and G474. The epitope preferably comprises less than 4, more preferable less than 3 amino acids of the more hypervariable region outside of the CD4bs, in contrast to the neutralizing antibodies b12, VRC01 and A12. The surface adjacent to the CD4bs is characterized by a high variability—due to high mutation rate—and this variability provides an escape to the virus from neutralization: escape mutants are created by changing the adjacent surface. Therefore, the smaller epitope that is recognized by the antibodies of the present invention results in more potent and broadly neutralizing antibodies. The invention therefore also provides an antibody that binds to amino acid residues D368 and I/V371 within the CD4 binding site of HIV IIIB gp120 protein, and which interacts with less than 4, more preferable less than 3 amino acids of a more hypervariable region outside of the CD4bs. Said interactions with amino acids of the hypervariable region are preferably weak interactions.

A further preferred epitope is outside of the CD4bs and comprises amino acid residues T388 and R419 of gp120, which is bound by antibodies of families L81H9 and L91B5. This epitope was not known before to be involved in binding to broadly neutralizing antibodies. The neutralizing antibodies b12 and 17b bind to R419 but not to T388. Therefore, the invention further provides an antibody that binds to amino acid residues T388 and R419 of gp120 outside the CD4 binding site of HIV IIIB gp120 protein. The antibodies, including the broadly neutralizing anti-HIV heavy chain variable domain antibodies identified herein, provide excellent tools for generating bi- or multispecific antibodies in combination with antibodies that bind to amino acid residues D368 and I/V371 within the CD4 binding site of HIV IIIB gp120 protein.

The invention further provides an antibody that effectively competes with an anti-HIV antibody of families L94D4, L91F10 and L92E7 for binding to its epitope on HIV particles. The term effectively is used to indicate that the competing antibody binds with substantially the same affinity to the epitope, when compared to the antibody of the invention. The term substantially is used to indicate that the difference in affinity between an antibody of the invention and a competing antibody is preferably less than 10-fold, more preferred less than 5-fold, more preferred less than 2-fold, more preferred less than 1.5 fold. A preferred competing antibody is capable of effectively competing with an antibody of the invention when the competing antibody lowers the affinity of the observed binding of an antibody of the invention to its epitope about 2-fold using the same molar amount of competing antibody. Assays for measuring competition are known in the art and include, for example, competitive ELISA.

### EXAMPLES

### Example 1

### Immunization and Generation of Display Libraries

1 Immunisation of Lama glama

All llama immunizations were approved and performed according to the guidelines of the Utrecht University Animal Ethical Committee (DEC number: 2007.III.01.013).

1.1a Immunisation of Lama glama with gp140UG37 and gp140CN54

Two Lama glama [designated as llama 8 and 9] were injected intramuscularly with mixture of gp140CN54 and gp140UG37, 50 μg of each protein was used in commercially available Stimune adjuvant (CEDI Diagnostics, Lelystad, The Netherlands). First boosting was given on day 7, with the same immunogen doses as in the first injection. The following booster injections were given on days 14, 21, 28, 35 and 113 with a mixture containing 25 μg of each gp140. Ten millilitres blood samples were collected at days 0 (before injection), 21 and 113. To construct immune libraries, 150 ml blood samples were collected at day 43 and 122.

To assess the llamas' immune response, MaxiSorp microtitre plates were coated with 50 μL gp140CN54, gp140UG37 or gp120IIIB [5 μg/mL] overnight at 4° C. After blocking with 200 μL 4% Marvel in PBS (MPBS) serial dilutions of pre-immune and immune sera were incubated for 1 h. Detection of bound llama single chain antibodies was performed by incubation with monoclonal antibody (mAb) 8E1 followed by peroxidase-conjugated goat anti-rabbit IgG (1:5,000 in 1% MPBS; Jackson Immunoresearch, West Grove, Pa., USA) all in 50 μL.

1.1b Immunisation of Lama glama with gp120Yu-2 109-428

Two Lama glama [designated as llama 17 and 23] were injected intramuscularly with 100 μg gp120Yu-2 109-428 in commercially available Stimune adjuvant. In total 4 injections were given on days 0, 14, 28 and 35 respectively. On days 0 and 28, 10 ml of blood was taken from both llamas to determine the immune response. 150 ml of blood was collected on day 45 for the construction of the libraries. The immune response was tested in the same way as described above, only the coating was done with gp120Yu-2 109-428.

1.2 Phage Library Construction

To construct immune libraries, 150 ml blood samples were collected on day 45 (gp120Yu-2 109-428 immunisation) or on day 122 (gp140UG37 and gp140CN54 immunisation), and peripheral blood lymphocytes (PBLs) were purified by Leucosep (cat 227290, Greiner Bio-One BV, The Netherlands). Total RNA was extracted from PBLs as described by (Chomczynski and Sacchi 2006, Nature Protocols 1 (2):581-585) and random primed complementary DNA (cDNA) was synthesised using SuperScript™ III First-Strand Synthesis System for RT-PCR (Invitrogen, cat. 18080-051). After purification of the cDNA with a QIAquick PCR Purification Kit (Qiagen, cat 28106), the cDNA was used as template for PCR using the combination of the leader and CH2 based primers (Verheesen et al. 2006, Biochimica Et Biophysica Acta 1764 1307-1319) which resulted in an amplification of the conventional and heavy-chain IgG repertoire gene fragments. Due to the lack of CH1 regions in heavy-chain antibodies, the amplified gene fragments of conventional and heavy-chain antibodies were separated on agarose gel. Subsequently, a SfiI restriction site was introduced upstream of FR1 in a nested PCR using the gel purified heavy chain amplicon as template. Since a BstEII restriction site naturally occurs in 90% of the FR4 of llama heavy-chain antibodies genes (Roovers et al. 2007, Cancer Immunology, Immunotherapy: CII 56:303-317) the repertoire of PCR-amplified genes was cut with BstEII and SfiI and the resulting 300-400 base pair (bp) fragments were purified from agarose gel. Finally cDNA fragments were ligated into a phagemid vector for display on filamentous bacteriophage (De Haard et al. 2005, Journal of Bacteriology 187 4531-4541) and electroporated in Escherichia coli (E coli)TG1 (K12, (lac-pro), supE, thi, hsdD5/F′traD36, proA+B+, ladq, lacZ_M15; Zymo Research, Irvine, Calif.). The rescue with helper phage VCS-M13 and polyethylene glycol precipitation was performed as described previously (Marks et al. 1991, Journal of Molecular Biology 222 (3) (December 5):581-597). Phage stock containing around 5×10¹¹ pfu/ml was prepared and used for subsequent biopanning. The libraries from all llamas, designated as Libraries 8, 9, 17 and 23 had sizes larger than 10⁷ individual clones and more than 90% inserts. Therefore these libraries can be considered as good.

### Example 2

### Isolation of anti-HIV VHH

2. The phage libraries were used for two different approaches to find VHHs that can neutralize HIV. We have developed a direct neutralization assay [2.1] and used the binding/selection strategy described before [2.2]

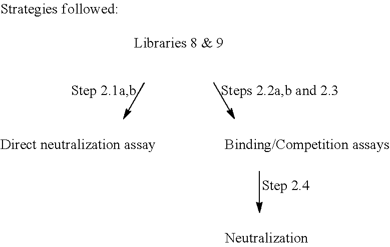

2.1a Isolation of Anti-HIV-1 VHH Through Direct HIV-1 Neutralization Screening.

Bacteria expressing the cloned VHH repertoire were plated onto agar containing 100 •g/ml ampicillin, 2% synctial stain (1 g methylene blue, 0.33 g basic fuschin in 200 ml methanol) and individual clones were picked using a Norgren CP7200 colony picker. Individual clones were expressed in TG1 E coli cells in a 96-well plate format. Each clone was expressed in 1 ml of 2×TY medium containing 100 •g/ml ampicillin and 0.1% glucose, followed by induction of VHH production with 0.1 mM isopropyl-β-D-thiogalactopyranosid (IPTG). The periplasmic extract was harvested from each well by filtration through a 0.2 uM PDVF membrane and screened for the ability to neutralize HIV-1. To enable high-throughput screening and characterization of VHH, neutralization was measured using 200 50% tissue culture infective doses of virus in the TZM-bl cell-based assay developed by Wei et al. (2002, Antimicrobial Agents and Chemotherapy 46:1896-1905) with Bright-Glo luciferase reagent (Promega). DNA from individual VHH which neutralized 2-6 viruses to less than 20% of RLU seen with a non-HIV specific VHH control was purified, sequenced and recloned into the pCAD51 expression vector followed by transformation into TG1 cells for purification and further characterization.

2.1b VHH Purification and Neutralization Profiling.

Expression from the pCAD51 vector incorporates a C terminal myc and 6-His tag (SEQ ID NO: 283) to the VHH and removes the bacteriophage gene III product. VHH were purified by means of the attached 6His-tag (SEQ ID NO: 283) using TALON Metal Affinity Resin (Clontech). The neutralization activity of the VHH was assayed in duplicate at either UCL or WMC laboratories. No virus inactivation was observed with a negative control VHH or with a pseudovirus bearing a rabies virus G-protein envelope or murine leukaemia virus envelope. VHH 50% inhibitory concentration (IC50) and IC90 titers were calculated using the XLFit4 software (ID Business Solutions).

This direct neutralization assay resulted in the detection of 24 neutralizing clones out of the 2816 tested VHH for library 8 and around 100 neutralizing clones out of 1056 tested VHH from library 9, demonstrating the quality of the libraries constructed and the excellent performance of the method developed. Examples of broadly neutralizing clones found by this method are L8Cj3 and L9Bm16 (clone of L93e3 to be described later). The broadness and the efficiency of one of the VHHs against infection by a number of different HIVs are given in Table 1. The amino acid sequence of this VHH and all other VHH are given in Table 2.

2.2a Selection of Clones Competing with sCD4 for Binding to gp140

To select phages that specifically bind to the CD4 binding site (CD4bs) of 0140 the modified competitive elution method (Forsman et al. 2008, Journal of Virology 82 12069-12081) using sCD4 as selective eluant was applied. Wells of MaxiSorp microtitre plates were coated with 100 μL gp140CN54 [2.5 or 0.5 μg/mL] in PBS overnight at 4° C. Blocking was performed with 4% Marvel in PBS (MPBS). After washing the plate with PBS, 5×109 phages, which were preincubated in 2% MPBS for 30 min at RT, were added to the wells and incubated for 2 hours at RT. Next, the coated wells were extensively washed with PBS. Subsequently, 100 μL sCD4 [30 μg/mL] or 100 μL triethylamine (TEA) 100 mM was added and the plates were incubated for 30 min at RT. The eluates were removed, the TEA eluted phage was neutralized with half volume of 1M Tris pH 7.5, and subsequently 10-fold serial dilutions in PBS were prepared. Ten microlitres of each dilution was used for infection of 190 μL log-phase E coli TG1. After infection at 37° C. for 30 min without shaking, 5 of bacterial suspension was spotted on LB agar plates supplemented with 100 μg/mL ampicillin and 2% glucose (LB/Amp100/Glu2%) to determine the enrichment of the first round. Moreover, 75 μL of eluate was used for infection 0.5 mL log-phase E coli TG1 to rescue phages (Marks et al. 1991, Journal of Molecular Biology 222 581-597), and were subsequently applied for second round of selection. The conditions of the following selection round were identical to the first one. These selections resulted in a large number of binders, notably VHH L91C2, L91F10, L91E1, L91B5, L91H9, L92D4, L91E2 and L92E7. These VHH were subsequently analyzed in enzyme-linked immunoabsorbent assay (ELISA) tests, sequences were determined (Table 2) and neutralization studies performed (see section 2.4).

2.2b Selection of Clones Using Modified gp120 Protein

In this selection process two rounds of selection were carried out. For the first round of selection, a MaxiSorp plate was coated overnight at 4° C. with 100 μl of serially diluted gp120Yu-2 109-428 (Chen et al. 2009, Science 326 (5956) 1123-1127) in sterile PBS and as a control only PBS was coated. The following concentrations were used 5 μg/ml, 1.67 μg/ml and 0.56 μg/ml. The plate was blocked with 4% MPBS for 1 hour. PEG precipitated phages, displaying VHH from library 8, 9, 17 or 23 were preincubated 1:10 in 2% MPBS, for 30 minutes before adding them to the blocked MaxiSorp plate which was washed with PBS/0.05% Tween 20 (PBST) for 3 times. The phages were allowed to bind to the Yu-2 109-428 for 2 hours while shaking at room temperature, before the plate was washed for 40 times with PBST to remove unbound phages. The plate was washed an additional 3 times with PBS to remove all remaining tween. Bound phages were eluted with TEA and rescued by infecting log phase TG1 bacteria.

The rescued output from the 5 μg/ml coated libraries was used to produce phages for the input of the second round. To purify the phages, PEG precipitation was performed. The method used in the second round of selection was similar to that of the first round, with a few exceptions, the plates were coated with 2 and 0.4 μg/ml gp120Yu-2 109-428 and the phages were preincubated 1:100 instead of 1:10. Next to a high pH elution, a competitive elution was done with a 50 times molar excess (compared to the coating) of b12, sCD4 or 17b. Also part of the plates were washed overnight with PBST at 4° C. and eluted for 6 hours. Examples of the clones found with this selection method are L81H9, L93E3 (clone of L9Bm16 isolated by direct neutralization assay) and L94D4. The selected clones were subsequently analyzed in ELISA, sequenced and tested in neutralization studies.

Interestingly, clones have been isolated that may bind to the CCR5 binding site (CCR5bs). It is well known that CD4 and b12 induce significant conformational changes of the gp120 envelope proteins (Chen et al. 2009, Science 326 (5956) 1123-1127). The Yu 109-428 gp120 has been developed using the knowledge of these conformational changes, in particular, so that the cavity below the bridging sheets is no longer assessable for antibodies, not even the small VHH. On the other hand the CCR5bs is more easily accessible in these gp120 and therefore the use of Yu-2 for selection may result in VHH recognizing the unchanged part of CD4bs and the CCR5bs. In the procedures applied herein 3 different wash strategies have been followed during the selection step, the non-specific TEA and the specific CD4 and b12. Surprisingly, the methods followed resulted in VHH that bind to the CCR5bs, i.e. L1719A12, L1720C1, L1720E4 and L2320F9. The sequences of these VHH are given in Table 2.

2.3. Screening ELISA

At the end of the second round, 100 μL serially diluted infected E coli TG1 were plated on LB/Amp100/Glu2% agar plates and single colonies were picked and grown in 2×YT broth containing 100 μg/mL ampicillin and 2% glucose (2×YT/Amp100/Glu2%) in 96-well microtitre plate format. Expression of the VHH from single clones was performed in 96 deep-well plates (cat.AB-0932, Westburg B.V, The Netherlands) according to the modified method described by (Marks et al. 1991, Journal of Molecular Biology 222:581-597). Briefly, 1 mL of 2×YT/Amp100/Glu0.1% broth was inoculated with 10 μL overnight culture and grown with shaking at 37° C. until OD600=1 was reached. Expression of the protein was induced by adding IPTG (final concentration of 1 mM) and the cultures were grown for an additional 4 hours shaking at 37° C. After harvesting bacteria by centrifugation for 15 min at 4566×g and freezing pellets overnight at −20° C., bacteria were resuspended in 100 μL PBS and shaken for 2 h at 4° C. Next, spheroplasts were harvested by centrifugation for 15 min 4566×g at 4° C. and supernatants (i.e. periplasmic fractions) containing VHH were taken for screening assays.

The clones that were selected with sCD4 elution from gp140UG37 were screened in the following setup. Periplasmic fractions were screened for their ability to interfere with binding of mAb b12 to gp140CN54 by direct competitive ELISA. This approach was chosen because of the weak interaction between gp140CN54 and sCD4 in our ELISA setup that prevented screening of individual clones for competition with sCD4. For this purpose, wells of MaxiSorp microtitre plates were coated with 50 μL b12 [2 μg/mL] in PBS overnight at 4° C. Next, the b12-coated wells were blocked with 4% MPBS for 1 hour. In the meantime, mixtures of 5-fold diluted periplasmic fractions and 1 μg/mL gp140CN54 (final concentration) in 1% MPBS were prepared and incubated for 1 hour at room temperature. Then 50 μL of each mixture was transferred into blocked, b12-coated wells and incubated for an additional hour. To detect bound, non-inhibited gp140CN54 biotinylated concanavalin A (ConA) was used at concentration 2 μg/mL in 1% MPBS followed by addition of HRP conjugated streptavidin (Jackson, Cat. No: 016-030-084). Complexes were detected as described above. Positive clones, which gave a low signal in the b12 competition assay, were selected and one-way sequencing was performed by application M13Rev primer (Verheesen et al. 2006, Biochimica Biophysica Acta 1764 1307-1319) (ServiceXS, Leiden, The Netherlands). VHH L91C2, L91F10, L91E1, L91B5, L91H9, L92D4, L91E2 and L92E7 were chosen for further characterisation. Therefore their VHH genes were recloned into the E coli (production vector and after expression the VHH were purified by means of immobilised metal affinity chromatography (IMAC) as it has been described by Verheesen et al. (Verheesen et al. 2003, Biochimica Biophysica Acta 1624 21-28).

The clones that were selected from the gp120 construct were screened in the following setup: MaxiSorp plates were coated overnight at 4° C. with 100 ng gp120Yu-2 109-428 in sterile PBS. The next day, the plates were blocked with 4% MPBS, shaking at room temperature, to prevent any non-specific binding. After three washes with PBST, 25 μl of VHH containing periplasmic fraction together with 25 μl 2% MPBS was added to each well and allowed to bind for 1 hour, shaking at room temperature. After 3 more washes with PBST, 50 μl of a set concentration sCD4, b12 or 17b (previously determined in a titration assay) was added to the plates, depending on the elution method used in the corresponding selection. The detection of sCD4 was done by 1:10.000 L120 (NIBSC, Cat. No: ARP35) followed by 1:5.000 HRP conjugated Donkey anti Mouse (Jackson, Cat. No: 715-036-151). b12 and 17b were detected with 1:10.000 Rabbit anti Human IgG (Dako, Cat. No: 0424), followed by 1:5.000 HRP conjugated Donkey anti Rabbit (Jackson, Cat. No: 711-036-152), or by 1:5.000 HRP conjugated Goat anti Human (Jackson, Cat. No: 109-035-088).

For all steps, the added proteins were allowed to bind for 1 hour, shaking at room temperature before washing three times with PBST. After the last step, the plate was washed with PBS, to remove all Tween remnants and the amount of HRP conjugated antibody was visualized by adding 50 μl o-Phenylenediamine which was substituted with 0.03% H2O2. Clones that gave a signal less than 30% of the signal of the control, were chosen for further characterization. These clones included VHH L81H9 for competition with b12, L93E3 for competition with sCD4 and L1719E1 for competition with 17b.

2.4. HIV Neutralization Assay

The HIV-1 neutralizing activity of the VHH were assessed in the TZM-bl cell based assay, as described previously (Forsman et al. 2008, Journal of Virology December; 82(24):12069-81). Briefly, 3-fold serial dilutions of purified VHH starting from 50 •g/mL were performed in duplicate in 10% (v/v) fetal calf serum (FCS) supplemented DMEM growth medium (Invitrogen, Paisley, UK). 200 TCID50 of virus was then added to each well and the plates were incubated for 1 hour at 37° C. TZM-bl cells were subsequently added (1×10⁴ cells/well) in growth medium supplemented with DEAE-dextran (Sigma-Aldrich, St Louis, Mo., USA) at a final concentration of 11 •g/mL. Assay controls included replicate wells of TZM-bl cells alone (background control), and TZM-bl cells with virus assayed (virus control). No virus inactivation was observed with a negative control VHH. Following 48 hours incubation at 37° C., all 100 •L of the assay medium was removed and 100 μL of Bright-Glo luciferase reagent (Promega, Madison, Wis., USA) was added to each well. The cells were allowed to lyse for 2 minutes, and the luminescence was then measured using a luminometer. The 50% inhibitory concentration (IC50) titres was calculated as the VHH concentration that achieved a 50% reduction in relative luminescence units (RLU) compared to the virus control RLU, after subtraction of the background control RLU from both values. The calculations were performed using the XLFit4 software (ID Business Solutions, Guildford, UK). The results of the neutralization studies of the selected VHH are given in Table 1. The amino acid sequences are given in Table 2.

### Example 3

### Analysis of Isolated VHH

3.1. In total 186 different sequences of VHHs that neutralize or recognize the CD4 binding site have been selected via the routes 2.1 and 2.2a,b-2.4. From these VHH the DNA sequences were determined according to standard techniques. These analyses resulted in the amino acid sequences given in Table 2.

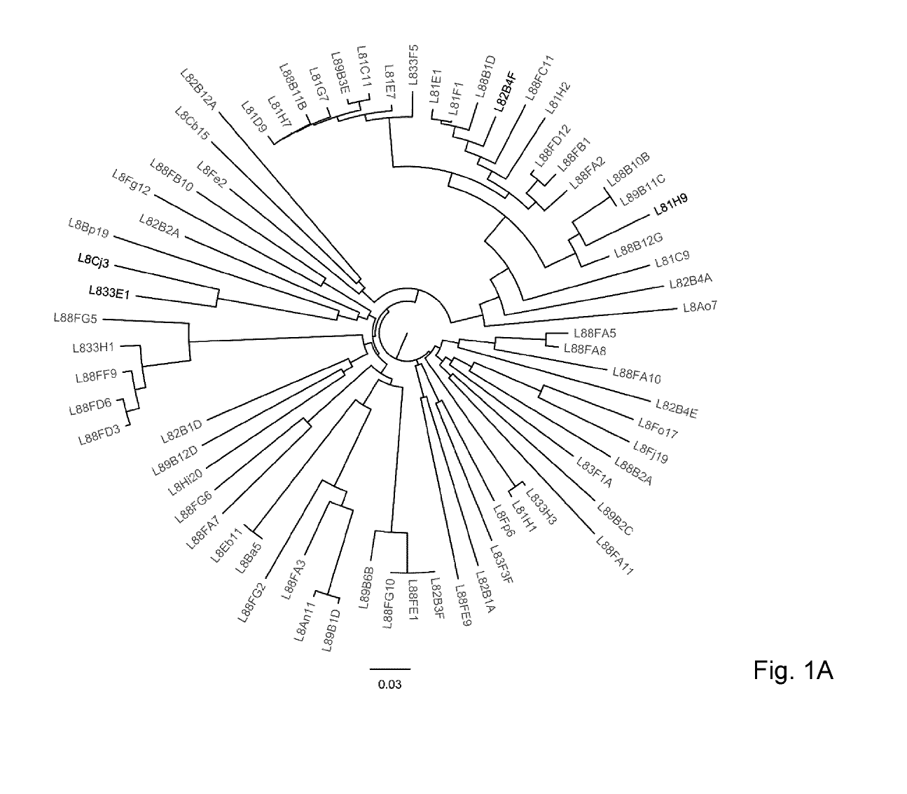

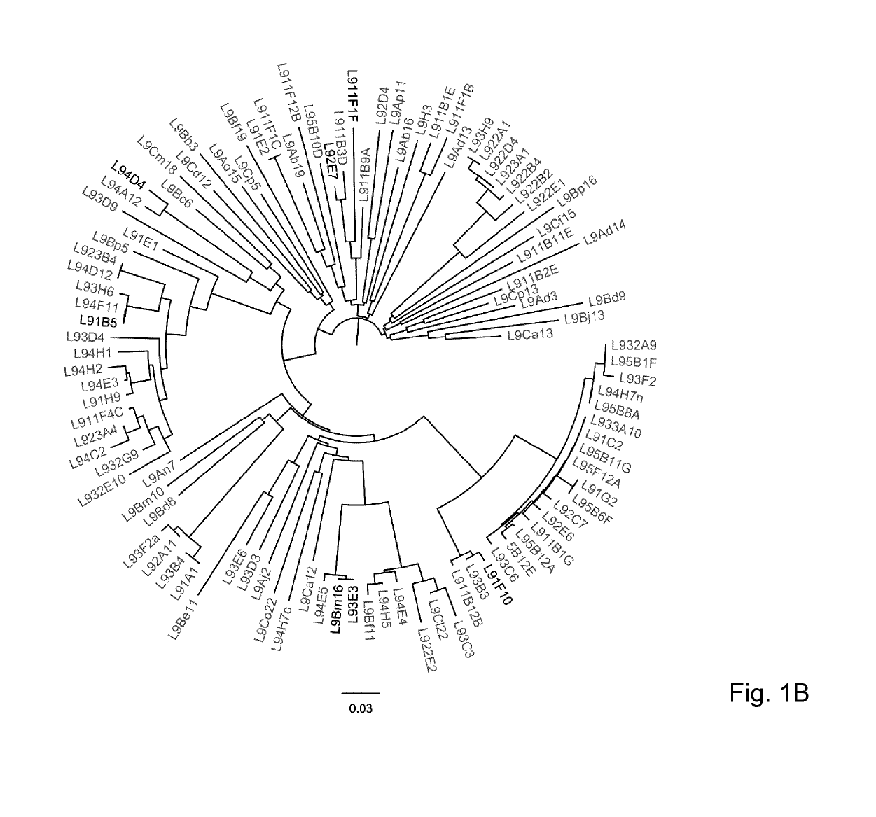

3.2 Most of these VHHs have been clustered using ClustalX analysis and the result of this clustering is given in a dendrogram, constructed by FigTree software (see FIG. 1).

3.3. Whereas the Clustal analysis resulting in the dendrogram depicted in FIGS. 1A and 1B simply aligned the sequences as accurately as possible, such clustering does not provide any information on the V-, D- and J genes from which these VHHs are matured. Based on literature data and DNA analysis of the area on which the V-, D- and J genes encoding VHHs are located on the Lama glama genome, the origin of the selected VHHs could be determined, as well which amino acids at a certain positions are generated during the maturation process. Although maturation is not a simple process resulting in ever better VHH, analysis of the amino acids that deviate from their germ line sequences is very informative to understand the structure/function relation of the VHH. Table 6 gives a maturation of a sub-set of the selected VHH that recognize the CD4bs of HIV.

Using the known V-, D-, J-genes of the Lama glama and Lama pacos genomes, the origin and maturation of the selected VHH were determined. The residues outside the complementarity determining regions (CDRs) that deviate from their germline sequence counterparts, and that have thus been altered during the maturation process, may play important roles in the function of the VHH. Table 6 shows a sequence alignment of a sub-set of the selected VHH that recognize the CD4bs of HIV, with the deviated residues outside the CDRs highlighted in light grey, and those inside CDR1 and 2 in dark grey.

3.4. Furthermore, inspection of the maturation process resulted in the observation that two sub-families have shortened CDR2s. Remarkably the family derived from Vs-t (Vt as determined on the DNA level) and the family derived from Vc-f (Ve as determined on the DNA level) genes, referred to as J3 and 3E3 family, respectively, all having CDR2s of 8 amino acids, resulting in these short CDR2s (Table 3). Therefore, this shortening was an active process during maturation of the VHHs. Inspection of about 1000 sequences of VHH showed that such deletions are extremely rare (1 in 1014 VHH selected against 30 different antigens). Finding two different families from two different llamas with such deletions has an even lower probability. This unexpected finding strongly indicates that these deletions are essential for the functionality of these VHH in the process of gp120 binding and subsequent neutralization of HIV.

3.5. The most important results of the bioinformatic analyses of all VHH (Table 2 and 3) resulted in the definition of 7 families of particular interest. The amino acid sequences of the CDR of these 7 families are given in Table 3. It is well known that during maturation the framework residues of the VHH are also mutated. In fact, a variability entropy analysis can be performed. It is obvious that the framework regions of the selected VHH described in this invention could be mutated using the variability entropy data (Lutje Hulsik et al 2009, Thesis Utrecht University Chapter 4 ISBN 978-90-393-5032-2).

### Example 4

### Isolation of Related Anti-HIV VHH by Family Approach

4. Family Approach to Enlarge the Number of VHH with Desirable Neutralization Properties

Having selected effectively neutralizing VHH and with the knowledge of their nucleotide sequences, it is feasible to analyze whether, in the RNA sample obtained from lymphocytes (see 1.1a and 1.1b), nucleotide sequences are available with the same 5′ and similar 3′ sequences (Koh et al 2010, Journal of Biological Chemistry, 285(25):19116-24).

In short the method used to construct L92E7 family library is as follows: VHH fragments that show similarity to L92E7 were amplified from the total library 9 with M13rev and a specially designed degenerative reverse primer* that recognizes the C terminus of the VHH, extending at least 5 amino acids into the CDR3. PCR was setup as follows, 2 min at 94° C., then 25 cycles of 30 s at 94° C., 1 min at 52° C., 2 min at 72° C., followed by a 7 min extension period at 72° C. The PCR product was run on a 1% TBE-ethidium bromide agarose gel and the VHH fragment corresponding 700 bp band was cut and extracted using QIAquick gel extraction kit (Qiagen, Cat. No: 28706). The VHH fragments were BstEII and SfiI digested, followed by another gel extraction in which the 350 bp band was cut and extracted with QIAquick gel extraction kit. The digested fragments are ligated overnight at 16° C., into a SfiI/BstEII digested pUR8100 phagemid vector with use of T4 ligase (Promega, Cat, No: M1801) and subsequently electroporated into E coli TG1 (K12, (lac-pro), supE, thi, hsdD5/F′traD36, proA+B+, lacIq, lacZ_M15). The transformed bacteria were titrated and plated onto LB-agar plates substituted with 2% glucose and 100 μg ampicillin to determine the library size (around 106) and to perform a colony PCR to determine the insert frequency of the VHH fragment (above 95%).

* M13rev forward primer:
(SEQ ID NO: 1)
5′-GAGCGGATAACAATTTCACACAGG
L92E7 specific reverse primer:
(SEQ ID NO: 2)
5′-TGAGGAGACGGTGACCTGGGTCCCCTGGCCCCAGTAGTC
NGARTANCG.

The same method was used to produce L8Cj3 and L93E3 family libraries, using the following primers:

L8Cj3 specific reverse primer:
(SEQ ID NO: 3)
5′-TGAGGAGACGGTGACCTGGGTCCCCYGGCCCCAGWMGTC
ATACYSATTTG.A
L93E3 specific reverse primer:
(SEQ ID NO: 4)
5′-TGAGGAGACGGTGACCTGGGTCCCCTGGCCCCAGGAMSC
AAADKCACGGAG.

The annealing temperature of the PCR reaction can be changed from 1 min at 52° C. to 60° C. to increase the specificity of the reaction and reduce the risk of finding unrelated VHH back in the library.

Table 10 shows neutralization data of L8Cj3 (J3) family members (Table 10A) and neutralization data of L93E3 (3E3) family members (Table 10B). Individual sequences of L8Cj3 (J3) family members are depicted in Table 11A. Individual sequences of L93E3 (3E3) family members are depicted in Table 11B.

### Example 5

### Isolation of Related Anti-HIV VHH by Molecular Evolution

5. Directed Evolution

In addition to the family approach, the directed evolution approach (Stemmer, 1994. Nature 370: 389-391; Stemmer, 1994. Proc. Natl. Acad. Sci. USA 91, 10747-10751) is undertaken to generate additional improved VHH, as was demonstrated by v. d. Linden et al 1999 (Thesis Utrecht University, Chapter 5 ISBN 90-646-4637-6). In particular, this method is useful if a large number of related VHH genes against a particular epitope are available, such as in this invention for the L81H9, L91B5, L91F10 and L93E3 families

### Example 6

### Identification of Binding Epitopes

6.1 Construction of 3D Model of One of the VHH of Table 2 and Determination of its Interaction with gp120

We determined the CD4 binding site by determining all residues that are buried by binding of CD4. In more detail, the surface accessibility of the gp120 structure alone was compared with the gp120-CD4 complexed structure. The obtained CD4 binding site differs slightly from the binding site derived from literature (Kwong et al. 1998. Nature 393: 648-659), because the GP120 was obtained from a virus of another subgroup.

We generated a 3D model of VHH L93E3 interacting with gp120 based on published crystal structures of classical antibodies in complex with gp120, and using our data concerning L93E3 (clone of L9Bm16, isolated simultaneously by methods 2.1 and 2.2b). L93E3 is a very broadly neutralizing VHH that competes with CD4 binding. L93E3 has an additional cysteine bridge to the one present in all VHHs, between the CDR3Cys100a and the framework Cys50, which stabilizes the conformation of the CDR3 loop of L93E3. Therefore the structure of L93E3 can be modeled better than the very broad neutralizing VHH L9Cj3 having a large flexible CDR3. A model of L93E3 was generated with the program Modeller 9.9 using the VHH D7 (Hinz et al., 2010, PLoS One Volume 5 Issue 5, e10482 1-7) as a template structure. This template structure does not have an additional cysteine bridge as in L93E3, so this bridge was not formed in the model either. Using the molecular building program Coot, the CDR3 loop was bent, so that the cysteine bridge was formed.

Prior to docking on gp120 using the program HADDOCK v2.1 (Dominguez, et al. (2003). J. Am. Chem. Soc. 125: 1731-1737), the geometry of the model of L93E3 was refined with CNS scrips, which are available in the HADDOCK program. The restraints in HADDOCK given were based on published data. In all antibody-gp120 complex crystal structures, two interactions seen in CD4 binding to gp120, seem to be key in binding of antibodies as well. Firstly, D368 always makes an interaction with a basic residue. Secondly, in the CD4-Phe43 binding pocket either a phenylalanine or a tyrosine binds at an equivalent position. We reasoned that L93E3, based on our biochemical and neutralization data, should also bind in a similar fashion. The CDR3 of L93E3 contains an arginine, a tyrosine and a phenylalanine. In HADDOCK two restraints were given. One restraint was that there had to be an interaction between the R100d in CDR3 and D368 of gp120. The other was that either Y100 or F100f should be in the CD4-Phe43 binding pocket. The best model in terms of HADDOCK scores had the Y100 in the Phe43-binding pocket and the arginine made a salt bridge to D368 of gp120. In the model of L93E3-gp120, L93E3 covers much of the previously determined epitope within the CD4bs (the black outline in FIG. 2C) and only minor parts are outside this site.

### Example 7

### Competition Assays

7. Competition Assays

The sequence analysis showed that a large number of quite different VHH have been selected. This analysis resulted in a number of families and sub-families on basis of their sequences. Subsequently we used competition assays to group the various sub-families of VHH.

7.1 Purified VHH were titrated against sCD4, b12 and 17b, to determine whether their respective epitopes overlap. For the competition assay, MaxiSorp plates were coated, overnight at 4° C., with 100 ng/well of gp140UG37, gp120IIIB or gp140CN54. The next day, the plates were blocked with 4% MPBS, shaking at room temperature, to prevent any non-specific binding. After three washes with PBST, 50 μl serially diluted VHH in 1% MPBS was added to the wells and allowed to bind for 1 hour, shaking at room temperature. After 3 more washes with PBST, 50 μl of a set concentration sCD4, b12 or 17b (previously determined in a titration assay) was added to the plates. The detection of sCD4, b12 or 17b was done as stated in section 2.3.

7.2 Subsequently, VHH that either came out of the direct neutralization studies (2.1) or binding (2,2b) were included in a large competition experiment among the VHH themselves to determine whether they had overlapping epitopes. To be able to detect only 1 of the two VHH that will be present during this assay, part of them had to be biotinylated. To do this a 10:1 molar ratio of NHS-LC-LC-biotin:VHH was used (Thermo scientific, Cat. No: 21343). This mix was incubated at room temperature for 1 hour. To remove any unbound biotin, the mix was dialyzed against PBS three times. To measure the optimal concentration of biotinylated VHH in the competition assay, these VHH were titrated for their binding to gp140UG37, gp120IIIB and gp140CN54. For the competition assay, MaxiSorp plates were coated, overnight at 4° C., with 100 or 250 ng/well of gp140UG37, gp120IIIB or gp140CN54. The next day, the plates were blocked with 4% marvel in PBS (MPBS), shaking at room temperature, to prevent non-specific binding. After three washes with PBST, 50 μl 100 μg/ml of the competing (non-biotinylated) VHH in 1% MPBS was added to the wells and allowed to bind for 1 hour, shaking at room temperature. This amount of VHH should be enough to cover all available molecules of the antigen, but to make sure that the competing VHH is actually binding, a binding assay in which the VHH was detected by anti Myc-tag (9E10) and HRP conjugated donkey anti mouse was performed in parallel. 10 μl of the biotinylated VHH was added to all of the wells, reaching the final concentration that was determined by the previously described titration. After an additional 1 hour incubation at room temperature, shaking, the plate was washed three times with PBST before 50 μl HRP conjugated Streptavidine (Strep-HRP) was added to the plates to detect the binding of the biotinylated VHH to the antigens. After washing three times with PBST followed by PBS, the amount of Strep-HRP was visualized by adding 50 μl o-Phenylenediamine which was substituted with 0.03% H2O2. The measured values from this assay were converted to percentages, in which the competition with itself was seen as the minimal binding (maximal competition), 0%, and the competition against an irrelevant VHH as the unhindered binding, 100%. An excerpt of the results of the competition assay is provided in Table 4.

### Example 8

### Construction of Mutant VHH

8.1 Construction of Mutant VHH to Increase their Efficiency, Broadness and Physical Stability.

Using the results of the maturation studies, comparison of various VHHs selected by any of the methods described in 2a-2d and a data bank of over 2000 VHH sequences and their physical properties like stability and the efficiency of their production in lower eukaryotes a number of mutations starting from a representative of each sub-family of VHHs that recognize the core CD4bs have been constructed using standard protocols. As example, an alanine scan was performed on residues V29, Y98, R100b, Y100c, Y102 of L92E7 by site-directed mutagenesis with application of the QuikChange® Site-Directed Mutagenesis Kit (Stratagene, Cat. No: 200518) according to the instructions of the manufacturer. The introduced mutations were verified by plasmid sequencing (ServiceXS, Leiden, The Netherlands). The mutated L92E7 are named L2E7, A1-A5 respectively and their sequences are provided in Table 2. The neutralization results of these 5 mutants were done on 4 viral strains and shown in Table 5. Further mutations will be made in various clones, like the R105Q for clones L8Cj3 and L833E1, which most likely will lead to better production. All mutations are shown in Table 6. A scheme for single mutations is given in Table 6. However it is clear that any combination of these single mutations can be made using the same protocol.

8.2 Construction of Mutant VHH to Study the Importance of Matured Residues and to Increase their Efficiency, Breadth and Physical Stability.

The L8Cj3 residues that deviate from the germ line that L8Cj3 is derived from were mutated back into the residues present in the germ line with the QuickChange Site-Directed Mutagenesis kit (Stratagene) according to the manufacturer's protocol. Thirteen mutations were produced (FIG. 6A) of which most were “reversal to germ line” mutations, except mutant 12, 8 and 9. Mutant 12 is the mutation of J3 from EL to IY in FR1. Some reversal to germ line mutations were already present in L8i5, an L8Cj3 family member, which neutralizes equally well as L8Cj3, therefore, these mutants were not made independently. J3r (see below) has some additional reversal to germ line mutations. Binding and neutralization of the purified L8Cj3 mutants were assessed (FIG. 6B). Mutations in the pre-CDR1 region impair binding to the subtype BC CN54 gp140 Env used to immunize the llama. A mutation in the pre-CDR2 region abolishes binding to the CN54 Env, impairs binding to the subtype A 92UG Env used to immunize the llama, and impairs neutralization of subtype A 92UG037 and subtype BC CH181 pseudoviruses (CN54 virus grows only as a primary isolate and stocks were not sufficient for testing, therefore the related BC CH181 virus was used). Most mutations in the framework 3 region impair binding to the CN54 gp140 Env. A mutation in the pre-CDR3 region (R95A) impairs binding to 92UG and CN54 Env trimers, and abolishes neutralization of 92UG037 and CH181 pseudoviruses.

A custom made gene for J3r was ordered from GeneArt/Life technologies and contained a few mutations with respect to L8Cj3 for cloning purposes and to increase the stability and the expression level of the VHH in Saccharomyces cerevisiae. These mutations are E1D, V5Q, F17S, S82aN and importantly R105Q (all numbering according to the Kabat numbering. The neutralization activity of J3r was not substantially different from that of L8Cj3 against a range of HIV subtypes (FIG. 6C).

8.3 Extending the CDR2 of L8Cj3 and L93E3 According to the Germ Lines Abrogate Binding to gp140 as Well as Neutralization on HIV-1

The QuickChange Site-Directed Mutagenesis kit (Stratagene) was used according to the manufacturer's protocol to introduce insertions in the CDR2 region of L8Cj3. Insertions in the CDR2 region of S, SW and SWS of L8Cj3 destroy the ability of the VHH to bind Env and neutralize pseudovirus (FIG. 6D).

Custom made genes for 3E3mod and 3E3mod-long-CDR2 were ordered at GeneArt/Life technologies. 3E3mod contains mutations V5Q, P14A, E82aN with respect to L93E3. 3E3mod-long-CDR2 contains the same mutations as 3E3mod with additionally the CDR2 filled in according to the germ line sequence (FIG. 6E). Reinsertion of the germ line sequences in the CDR2 of L93E3 abrogated binding to its epitope, similar to reinsertion of the germ line sequences in the CDR2 of L8Cj3 (see FIG. 6F).

### Example 9

### Construction of Bispecific Biheads

9. Construction of Bispecific Biheads

For microbicides it is important to contain very broad and efficacious neutralizing agents. Although the monovalent VHH described in this invention are extremely broad and neutralize potently, both characteristics can be further improved by constructing biheads made from 2 non-competing monovalent VHH. Having more than one binding domain in one molecule may increase its potency significantly, therefore it is necessary to have more than one VHH capable of recognizing the antigen, however they must not interfere with each other's binding. Table 4 provides the information about which VHH recognize different, non-overlapping epitopes and therefore can be used to construct bispecific VHH. As controls, we will also construct bispecific VHH consisting of VHH that recognize (partially) overlapping epitopes. Another reason to construct biheads is that from the four viral strains that are not neutralized by L8Cj3 (620345.C1; CAP45.2.00.G3; Du172.17; X2160.c25), at least two are being neutralized by a L92E7 family member (L911F1F) and at least one is neutralized very potently by L81H9 (X2160.c25 is only tested for L8Cj3). Linking L8CjJ3 to L911F1F or L81H9 may even increase the breadth of the already extremely broad VHH. Using methods well described in the literature a number of bispecific bi-heads have been constructed. In order to generate bispecific VHH that are connected with a 15 amino acid 4Gly/Ser (SEQ ID NO: 284) linker, the N and C terminal fragments need to be generated in separate PCR reactions:

For the N Terminal Fragments:

A PCR was performed using the DreamTaq green (Fermentas, Cat. No: EP0712), 0.5 μl of the bacterial glycerol stock (VHH in pAX51 vector) was used as DNA template, 2.5 μl of a 5 μM stock of primers M13 rev forward primer and R5GSBam reverse primer was used. The following PCR setup was used: 5 min at 95° C., then 34 cycles of 30s at 94° C., 30s at 55° C., 45s at 72° C., followed by a 10 min extension period at 72° C. The PCR product was cleaned using the NucleoSpin® Extract II kit, PCR clean-up protocol, (Machery-Nagel, Cat. No: 740609.250) and eluted in 26 μl. 3 μl Buffer Green3 and 1 μl SfiI was added and the mix was incubated at 50° C. for 90 min. 1 μl BamHI was added and the mix was incubated an additional 90 minutes at 37° C.

For the C Terminal Fragments:

The protocol used for the C terminal fragments is similar to that of the N terminal ones, except that primers F10GSBam forward primer and M13for reverse primer were used. Further, the elution from the PCR clean-up was done in 22 μl and 6 μl of buffer Tango3 was added together with 1 μl BamHI and 1 μl Eco91I and incubated at 37° C. for 3 hours. The digested fragments were run on a 1% agarose gel and the 350 bp band that corresponds with the VHH was excised and purified using NucleoSpin® Extract II kit, gel extraction protocol. The N and C terminal fragments were ligated for 1 hour at room temperature in a 1:1 ratio into SfiI/NcoI/Eco91I digested pAX51 vector using T4 DNA Ligase (Fermentas, Cat. No: EL0011). The construct was then transformed into chemically competent E coli TG1 and subsequently plated onto LB agar plates substituted with 2% glucose and 100 μg ampicillin. In the above protocol the linker length can be varied by the use of different primers that anneal to the C terminus of the N terminal fragment and the N terminus of the C terminal fragment. The linker can be as long as 35 amino acids.

M13rev forward primer:
(SEQ ID NO: 1)
5′-GAGCGGATAACAATTTCACACAGG
R5GSBam reverse primer:
(SEQ ID NO: 5)
5′-AGTAGGATCCGCCACCTCCTGAGGAGACCGTGACCTGGG
TCCC

All digestion enzymes and buffers were from Fermentas. BamHI, Cat. No. ER0051; Eco91I, Cat. No. ER0391; SfiI, Cat. No. ER1821; The buffers came with the enzymes.

F10GSBam forward primer:
(SEQ ID NO: 6)
5′-TCTTGGATCCGGCGGGGGAGGTAGTGGGGGTGGGGGCTC
AGAGGTGCAGCTGGTGGAGTCTGGG
M13for reverse primer:
(SEQ ID NO: 7)
5′-GCCAGGGTTTTCCCAGTCACGA

The above protocol is exemplified by the use of L93E3 as N terminal fragment and L81H9, L91F10, L92E7 and L94D4 as C terminal fragment with use of the 15GS linker.

Table 7 gives a survey of the bispecific VHH that are or can be constructed.

Preferred bispecific biheads are constructed from two non-competing monovalent VHH. Groups of non-competing VHH are indicated in FIG. 7.

### Example 10

### Construction of Therapeutic Agents Comprising the Selected VHH

10. Construction of Therapeutic Agents Using Some of the Selected VHH as the Binding Domain of these Agents

Whereas for microbicides mono- or bi-head VHH are most suitable and economically feasible (Gorlani et al., (2012). AIDS Research and Human Retroviruses, 28, 198-205), for therapeutic use of the selected VHH, other criteria are very important. VHH have several disadvantages compared to conventional antibodies when used as therapeutic agents. One of the disadvantages is the relative short plasma half life (about 6 hours) in humans. It has been demonstrated that bi- and tri-heads have longer half lives, but still it is much less than the half live of IgG1, which is about 2 weeks. One of the improvements is the construction of bi-heads that interact with the abundant antigens on the surface of cells, provided that such interaction does not result in internalization of the bi-head, or that it facilitate an interaction with IgG1.

Another improvement that could extend the half life of VHH is to couple them to the Fc part of human IgGs, in particular the Fc region of IgG1. Another considerable disadvantage of VHH is the lack of interaction with the human immune system. As has been shown convincingly for mAb b12 (Moldt B et al., 2011. J Virol 85, 10572-81; Moldt B et al., 2012. J. Virol. 86, 6189-6196) that the constant domains of conventional (human) antibodies are necessary to neutralize viruses in the blood of patients. In that way the effector functions utilize intrinsic immunological mechanisms to destroy target cells and free viruses. Adding an IgG1 interacting VHH into a bi- or tri-head, or coupling an Fc part to the VHH or a bi- or trihead comprising the VHH, will make sure that the intrinsic immunological mechanisms are also activated. Therefore we have used molecular biological techniques, together with the sequence data of human and llama conventional antibodies to make the constructs depicted in FIG. 3 and FIG. 5.

10.1 in Short the Construction of One of these Chimeric Molecules, Using VHH L93E3 is Carried Out as Follows:

L93E3 was directly fused to the llama Fc of IgG2 or of IgG3 (hinge-CH2-CH3) via the method described in patent application WO 2005/037989, but avoiding the inserted residues AspGln (DQ) by using splicing-by-overlap extension PCR (see FIG. 3B). In this way heavy chain antibody encoding constructs were made in pcDNA3.1 or comparable vectors for transient expression in HEK293(E) cells or other mammalian cells like CHO or COS. Alternatively a chimeric heavy chain construct can be generated by using the llama hinge region and the Fc of human IgG1 (hinge-CH2-CH3) as was described by Zhang et al. (Zhang et al. 2009, Protein expression and purification 65(1):77-82). The vector system used by Zhang and colleagues is shown in FIG. 3. The same procedure was followed to construct fusions with L8Cj3, L81H9, L91B5, L92E7, L911F1F and L91F10

It is well described in the literature that the effector function can be improved (Kubota et al 2009, Cancer Science 9, 1566-1572). The constructs depicted in FIG. 3 are optimized accordingly. Moreover, to enhance the ADCC route, the •FUT8 CHO cells (Yamane-Ohnuki et al 2004, Biotechnol. Bioeng 87, 614-622) are used to produce these chimers. Using similar routes the CDC function mediated by Fc domains can also be enhanced.

10.2.1

In addition, L8Cj3, L93E3, L92E7 and L911F1F were directly fused to wild type human Fc of IgG1 including the hinge and CH2 and CH3 domains via the method described in patent application WO 2005/037989 (see also FIGS. 3 and 5). In addition, L8Cj3, L93E3, L92E7 and L911F1F were fused to the Fc of IgG1 with enhanced ADCC function (IgG1 Fc containing the mutations S239D and I332E (see FIG. 5). The ADCC effector function was improved according to the methods described in Kubota et al. 2009, Cancer Science 9, 1566-1572. The sense primer, prVHH(s), 5′-CGGTCGTCTCACTCTGAGGTGCAGCTGGTGGAGTC-3′ (SEQ ID NO: 8) and the anti sense primer prVHH(as) 5′-CGGTCGTCTCTGGTTCTGAGGACACGGTGACCTG-3′ (SEQ ID NO: 9) were used to produce the PCR fragment of the VHH. PCR fragments were ligated into a mammalian expression vector containing a signal sequence. Large plasmid preparations were performed using the QIAGEN Plasmid Maxi Kit according to the manufacturer's protocol. In this way heavy chain antibody encoding constructs were made for transient expression in cells. All VHH-Fc were produced by transient transfection in HEK293(E) and purified using Protein A affinity chromatography. The binding of the VHH-Fc fusions to gp140-UG037 was confirmed by titration ELISA and the binding of the VHH-Fc to the FcγRIII (CD16) was tested with ELISA. As expected, the VHH fusion with wild type Fc and with ADCC enhanced Fc bound well to the FcγRIII, whereas the VHH fusion with ADCC deficient Fc did not bind.

10.2.2 Measurement of ADCC Potency of VHH-Fc Fusions.

The potency of ADCC of all of the above described Fc fusions, as well as ADCC silenced versions, was determined in a luciferase-based NK killing assay adapted from Moldt et al. (Moldt et al., 2011. J Virol 85, 10572-81). In short: target CD4+ T cells were purified and infected with HIV encoding Renilla luciferase (Edmonds et al., 2010. Virology 408: 1-13). After 4 days of infection, the cells were mixed with VHH-fc fusions or VHH at different concentrations and with purified NK CD16+ effector cells from the same donor at an effector-to-target cell ratio of 5:1. After 7 hours incubation, the percentage of cells killed was calculated by measuring the luminescence expressed within live but infected target cells. The potency was determined as the concentration of antibody giving 50% of maximal degree of cell killing. Typically the potency of cell killing was better for antibody versions containing the glyco-engineered Fc or containing mutations responsible for high affinity binding to CD16 (Fc gamma IIIa receptor), whereas the Fc fusions from human and llama IgG1 gave intermediate potencies and lower levels of cell killing, and the VHH-Fc constructs with silencing mutations only gave minimal to no cell killing as indicated in Table 12.

10.3 ADCC Mediated Killing of gp120 Expressing Cell as Determined in Ex Vivo Studies

The capability of clearing HIV infected cells, which express the viral envelope protein gp120 on their surface, was measured with full blood samples from HIV positive donors. In addition blood samples from healthy volunteers were taken and spiked with (10⁴ to 10⁵ cells) CHO cells expressing gp120 on the cell membrane. Heparinized whole blood (150 μL) was incubated with a concentration range of VHH-Fc fusions (or neutralizing mAb b12) for 2 days at 37° C. and 5% CO2 in a final volume of 250 μL. Target cells were stained with FITC labeled anti-gp120 and with other antibodies labeled with another fluorescent dye by incubating for an additional 30 minutes. Following lysing of erythrocytes, the number of target cells was determined in FACS. Their depletion as a consequence of ADCC induced by VHH-Fc was determined in relation with the used concentration of the Fc fusion. In this way the potency of killing gp120 expressing CHO cells was determined using the blood samples of healthy individuals and compared with the capacity of the VHH-Fc constructs to induce killing of the gp120 positive cells in blood of HIV positive individuals.

### Example 11

### Construction of Diagnostic and Prophylactic Agents Comprising the Selected VHH

11. Imaging and Apherese Applications of the Selected VHH (Brummelhuis et al. (2010). Shock 34(2):125-32)

There are a number of applications besides the usage of the selected VHH as prophylactic or therapeutic agent. Some examples are given below.

11.1 The determination of binding kinetics and binding constants of therapeutic agents to proteins encoded by HIV is very important. This can be assessed by surface plasmon resonance (SPR) and the results of SPR experiments with a sub-section of the VHH are summarized in Table 8, including the extremely high binding constant (KD) of individual VHH for HIV-1 envelope proteins. Briefly, these experiments were carried out as per the following method: 100 ug/mL stocks of VHH, mAb and CD4 were prepared in [symbol] IgG2 labeled with Alexa 594. 4C9 anti p17 mouse antibody was visualized using goat anti-mouse IgG2 labeled with Alexa. The labels of anti-p17 antibodies and anti env J3 VHH clearly overlap with each other. The same observation was made using 3E3 VHH.

Electron-microscopy images of HIV-1 Bal infected MDM cells are provided in FIG. 10. At various magnifications the gold labeled HIV particles are clearly located in cell organelles, probably Trans Golgi structures.

### Example 12

### L8CjJ3 Neutralizes all Chimeric Simian-HIV (SHIV) Strains Tested

### Materials and Methods

Molecularly cloned SHIV 89.6p and HIV-1 89.6 were obtained from J. Sodroski (Dana-Farber Cancer Institute, Boston, Mass.) through the NIH AIDS Research and Reference Reagent Program. HIV1157ipEL-p and a molecular clone of SHIV1157ipd3N4 were provided by R. Ruprecht (Dana-Farber Cancer Institute), and SHIVSF162p3 was a gift of C. Cheng-Mayer (Aaron Diamond AIDS Research Center, New York, N.Y.). SHIVSF162P4 was obtained from the Division of AIDS, MAID, NIH. Pseudotyped SHIV viruses were prepared by E. J. Verschoor and Z. Fagrouch (Biomedical Primate Research Centre IBPRC), Rijswijk, Netherlands) essentially as described by Wei et al. (Wei et al. 2002. Antimicrob Agents Chemother 46: 1896-1905). In short, the full-length env genes were amplified from molecularly cloned viruses or from virRNA, and the PCR products were cloned into the expression plasmid pcDNA3.1 (Life Technologies). Individual clones were sequenced and selected for their suitability to produce pseudoviruses in a small-scale infection assay on TZM-bl indicator cells before performing neutralization assays (Montefiori, 2005. Current Protoc Immunol Chapter 12: 12-15). For this purpose, small stocks of pseudotyped viruses were prepared by transfection of 293T cells with a mixture of the pcDNA-env plasmid and the pSG3denv plasmid, which contains an Env-deficient molecular clone of HIV-1 SG3 (Kirchherr et al. 2007. J Virol Methods 143: 104-111). After incubation, cell-free virus stocks were produced by low-speed centrifugation, followed by filtration through a 45-μm filter, and used to infect TZM-bl cells. Viruses that induce luciferase activity were selected for the pseudovirus neutralization assay. The neutralization activity of the VHH was assayed in duplicate at the BPRC laboratory in the TZM-bl cell-based assay as described (Derdeyn et al. 2000. J Virol 74: 8358-8367; Wei et al. 2002. Antimicrob Agents Chemother 46: 1896-1905; and Li et al. 2005. J Virol 79: 10108-10125), containing 15 μg/ml DEAE-Dextran, and assayed with Britelite Plus Reagent (PerkinElmer) according to manufacturer's instructions using a Victor light plate reader (PerkinElmer). VHH IC50 titers were calculated using the Luc5 Samples02-NotProtected.xls program (courtesy of D. Montefiori). The derivation of SHIV11571PD3N4 and SHIV11571P EL-p was described in detail in Humbert et al. (Humbert et al. 2008. Retrovirology 5: 94). The subtype B SHIVs assays were undertaken once, and the subtype C SHIVs were assayed in two independent experiments.

### Results

L8Cj3 was found to potently neutralize six SHIV pseudoviruses from subtypes B and C, with IC₅₀ values all <0.5 μg/ml (see Table 13). The Glade C SHIVs were in fact potently neutralized with IC₅₀ values of <0.02 μg/ml. The strains assayed included one derived from SHIV11571P EL-p, a Glade C SHIV strain which has been used in recent mucosal challenges in NHPs (Humbert et al. 2008. Retrovirology 5: 94), and SHIV11571PD3N4 (Table 13), a highly replication-competent, mucosally transmissible Glade C R5SHIV which rapidly induces abnormalities in immune parameters and could therefore be used to assess post-acute viremia levels as readout parameters of vaccine or microbicide efficacy (Song et al. 2006. J Virol 85: 8954-8967).

### Example 13

### Selection of Three Neutralizing VHH, VLP1_A14, VLP3_b21 and VLP1_b9, from a Llama Immunized with DNA and VLPs

Two llamas (#1 and #3) were immunized via intramuscular injections, according to schedule 1. Genes encoding gp145 of the HIV-1 strains R2 and of 96ZM were cloned into the mammalian expression vector pcDNA3.1. The gene encoding R2 was inserted between the NheI and PmeI restriction sites and the gene encoding 96ZM was inserted between the NheI and XhoI restriction sites. A large scale DNA preparation was performed to obtain at least 60 mg of DNA. 200 μg of R2 virus like particles (VLPs) and 200 ug of 96ZM VLPs were made. These pseudotyped chimeric VLPs were made as described by Ludwig and Wagner (2007) Current Opinion in Biotechnology 18(6), 537-545. Purified gp120 from R2 and from 96ZM were mixed with Stimune commercially available Stimune adjuvant (CEDI Diagnostics, Lelystad, The Netherlands).

To construct immune libraries, 150 ml blood samples were collected. Phage library construction was performed as described in Example 1.2. VHH were isolated through direct HIV-1 neutralization screening as described in Example 2.1a. VLP1_A14 and VLP1_b9, which are inter-related family members, were isolated from the library from llama #1, and VLP3_b21 was isolated from the library from llama #3. Neutralization was tested against a range of HIV subtypes. All three neutralized over 70% of all tested subtypes (Table 14).

Schedule 1

| Day | Immunogen | Adjuvant | Sample |
|---|---|---|---|
| 0 | intramuscular injection of plasmid DNA | No | 10 ml |
| | encoding R2 and 96ZM gp145 in PBS | | blood |
| | (7.5 mg of DNA) | | |
| 14 | intramuscular injection of plasmid DNA | No | |
| | encoding R2 and 96ZM gp145 in PBS | | |
| | (7.5 mg of DNA) | | |
| 28 | Intramuscular injection of plasmid DNA in | No | 10 ml |
| | PBS (7.5 mg of DNA) | | blood |
| | & VLPs bearing R2 and 96ZM envelope | | |
| | proteins in PBS (protein content 50 μg) | | |
| 35 | Intramuscular injection of plasmid DNA in | No | |
| | PBS (7.5 mg of DNA) | | |
| | & VLPs bearing R2 and 96ZM envelope | | |
| | proteins in PBS (protein content 50 μg) | | |
| 42 | VLPs bearing R2 and 96ZM envelope | No | 10 ml |
| | proteins in PBS (protein content 50 μg) | | blood |
| 49 | VLPs bearing R2 and 96ZM envelope | No | |
| | proteins in PBS (protein content 50 μg) | | |
| 54 | n/a | n/a | 150 ml |
| | | | blood |
| 131 | Intramuscular injection of ZM96 & R2 gp120 | Stimune | 10 ml |
| | protein (50 μg each) | | blood |
| 145 | Intramuscular injection of ZM96 & R2 gp120 | Stimune | |
| | protein (50 μg each) | | |
| 159 | Intramuscular injection of ZM96 & R2 gp120 | Stimune | 10 ml |
| | protein (50 μg each) | | blood |
| 166 | Intramuscular injection of ZM96 & R2 gp120 | Stimune | |
| | protein (50 μg each) | | |
| 174 | n/a | n/a | 150 ml |
| | | | blood, |
| | | | used for |
| | | | library |
| | | | gener- |
| | | | ation |

### Tables

TABLE 1
Summary of neutralization data.

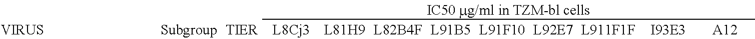

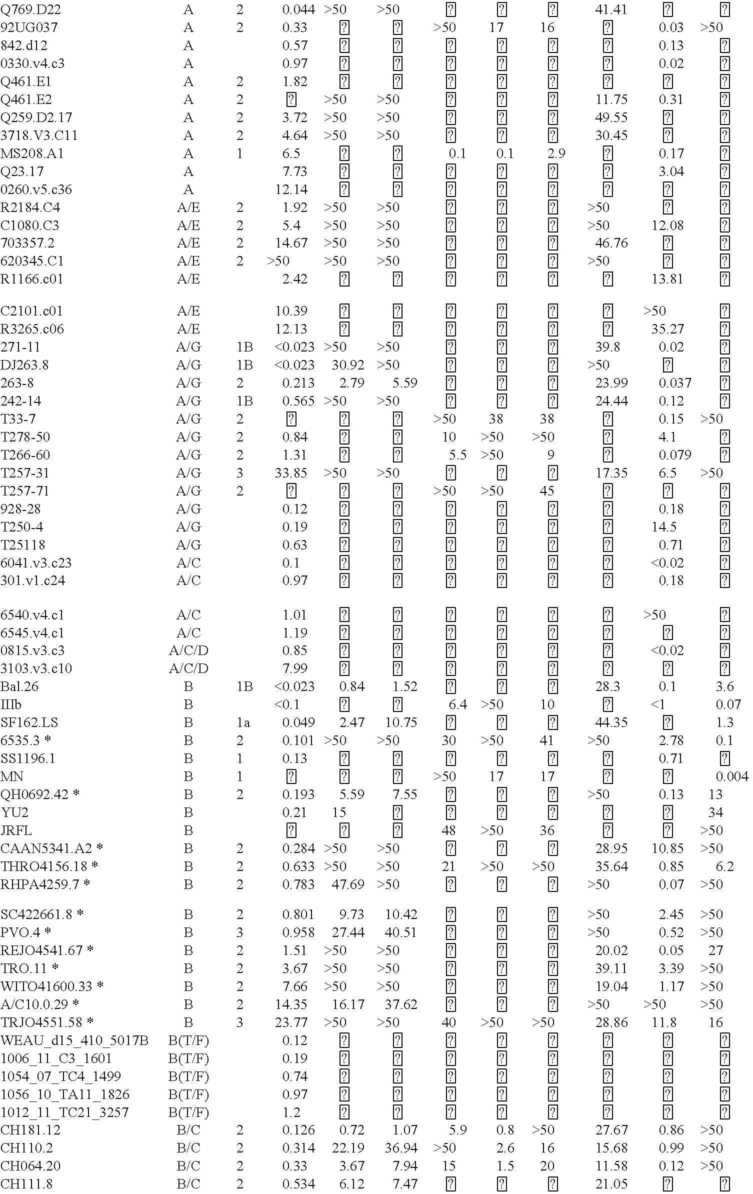

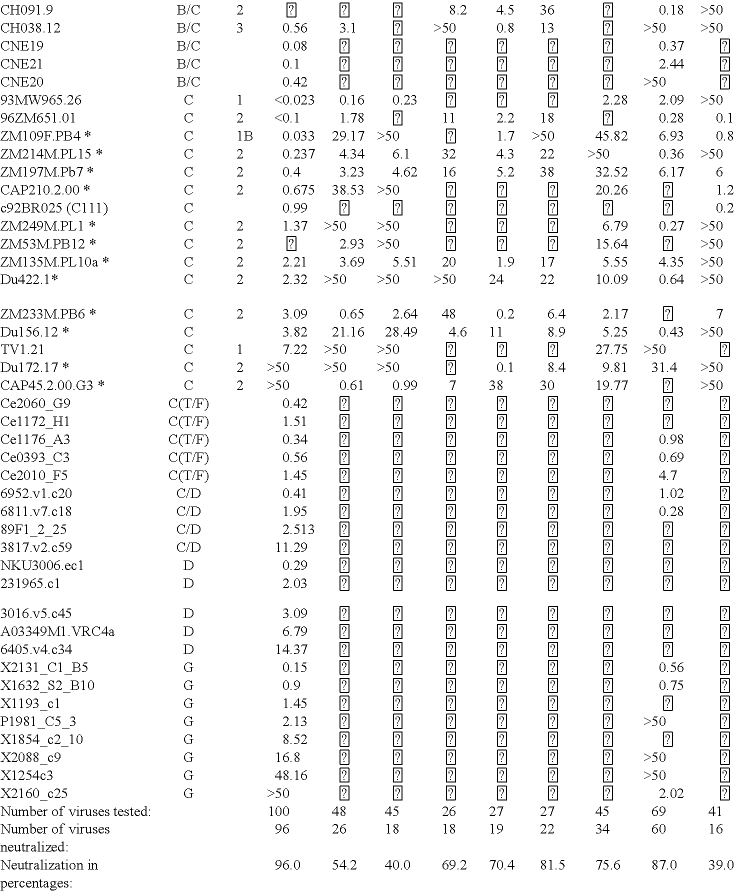

IC50 values from VHH clones against various HIV-1 strains given in μg/ml.
Names of the viral strains are given, as well as the subgroup and the tier category (if known) they belong to.
Viruses belonging to the B and C reference panels (Li, et al. (2005) J Virol. 79(16): 10108-25. and Li et al. (2006) J Virol. 80(23): 11776-90) are marked with *.
Values below 1 μg/ml are marked in dark grey, between 1 and 10 μg/ml in medium grey, between 10 and 50 μg/ml in light grey.
>50 means that the strain was not neutralized at the highest concentration tested, • means that the combination was not tested.

TABLE 2
Amino acid sequences of all
anti-HIV VHH. List of amino acid sequences
of all anti-HIV VHH as describe
herein. The members of the 7 most
important families are grouped at
the top of the list, followed by
the sequences derived from the family
approach and the mutational studies.
The other VI-LH are shown underneath.

L81H9 family
>L81D9
(SEQ ID NO: 10)
EVQLVESGGGLVQAGGSLGLSCAASGFTSDDYAIGWFRQAPGKE
REGVSYISHEDGSTYVADSVRGRFTISYDYAKNTVYLQMNSLKP
EDTAVYYCAAAILGNSVLWLWEYEYRYWGQGTQVTVSS
>L81H7
(SEQ ID NO: 11)
EVQLVESGGGLVQAGGSLRLSCAASGFTSDDYAIGWFRQAPGKE
REGVSYISHEDGSTYVADSVRGRFTISYDYAKNTVYLQMNSLKP
EDTAVYYCAAAILGNSVLWLWEYEYRYWGQGTQVTVSS
>L88B11B
(SEQ ID NO: 12)
EVQLVESGGGLVQAGESLRLSCAASGFTSDDYAIGWFRQAPGKE
REGVSYISHEDGSTYVADSVRGRFTISYDYAKNTVYLQMNSLKP
EDTAVYYCAAAILGNSVLWLWEYEYRYWGQGTQVTVSS
>L81G7
(SEQ ID NO: 13)
EVQLVESGGGLVQPGGSLRLSCAASGFTSDDYAIGWFRQAPGKE
REGVSYISHEDGSTYVADSVRGRFTISYDYAKNTVYLQMNSLKP
EDTAVYYCAAAILGNSVLWLWEYEYRYWGQGTQVTVSS
>L89B3E
(SEQ ID NO: 14)
EVQLVESGGGLVQAGGSLRLSCAASGFTSDDYAIGWFRQAPGKE
REGVSYISHEDGSTYVADSVRGRFTISYDYAENTVYLQMNSLKP
DDTAVYYCAAAILGNSVLWLWEYEYRYWGQGTQVTVSS
>L81C11
(SEQ ID NO: 15)
EVQLVESGGGLVQAGGSLRLSCAASGFTSDDYAIGWFRQAPGKE
REGVSYISHEDGSTYVADSVGGRFTISYDYAENTVYLQMNSLKP
DDTAVYYCAAAILGNSVLWLWEYEYRYWGQGTQVTVSS
>L81E7
(SEQ ID NO: 16)
EVQLVESGGGLVQAGGSLRLSCAASGFTSDDYAIGWFRQAPGKE
REGVSYISHEDGSTYVADSVRGRFTISSDYAKNTVYLQMNSLKP
EDTAVYYCAAAILGNSVLWLWEYEYRYWGQGTQVTVSS
>L833F5
(SEQ ID NO: 17)
EVQLVESGGGLVQAGGSLRLSCAASGFTSDDYAIGWFRQAPGKE
REGVSYISLEDGSTYVADSVRGRFTISYDYAKNTVYLQMNSLKP
EDSAVYYCAAAILGNSVLWLWEYDYRYWGQGTQVTVSS
>L81E1
(SEQ ID NO: 18)
EVQLVESGGGLVQAGGSLRLSCAASGFTSDDYAIGWFRRAPGKE
REGVSYVSIEDGSTYTADSVKGRFTISSDNAENTVYLQMNSLKP
EDTAVYYCAAAILGDYVRWLQEYDYDYWGQGTQVTVSS
>L81F1
(SEQ ID NO: 19)
EVQLVESGGGLVQAGGSLRLSCAASGFTSDDYAIGWFRRAPGKE
REGVSYISIEDGSTYTADSVKGRFTISSDNAKNTVYLQMNSLKP
EDTAVYYCAAAILGDYVRWLQEYDYDYWGQGTQVTVSS
>L88B1D
(SEQ ID NO: 20)
EVQLVESGGGLVQTGGSLRLSCAASGFTSDDHAIGWFRRAPGKE
REGLSYISIEDGSTYTADSVKGRFTISSDNAKNTVYLQMNSLKP
EDTAVYYCAAAILGDYVRWLQEYDYDYWGQGTQVTVSS
>L82B4F
(SEQ ID NO: 21)
EVQLVESGGGLVQAGGSLRLSCAASGFTSDDYAIGWFRRAPGKE
REGVSYISIEDGSTYSADSVKGRFTISSDYAKNTVYLQMNGLKP
EDTAVYYCAAAILGDSVRWLQEYDYDYWGQGTQVTVSS
>L88FC11
(SEQ ID NO: 22)
EVQLVESGGGLVQPGGSLRLSCAASGFTSDDYAIGWFRRAPGKE
REGVSYISNEDGSTYHADPVKGRFTIYSDNAKRTVYLQMNSLKP
EDTAVYYCAAATLGDYVRWLSEYDYNYWGQGTQVTVSS
>L81H2
(SEQ ID NO: 23)
EVQLVESGGGLVQAGGSLRLSCAASGFTSDDYAIGWFRRAPGKE
REGVSYISIEDGSMYYANSVKGRFTISSDNAKKTVYLQMNSLKV
EDTAIYYCAAAIFGDSVRWLSEYEYNYWGQGTQVTVSS
>L88FD12
(SEQ ID NO: 24)
EVQLVESGGGLVQAGGSLRLSCAASGFTSDDYAIGWFRQAPGKE
REGVSYISAEDGSTYNADSVKGRFTISSDNAKNTVYLQMNSLKP
EDTAVYYCAAALLGDSVRWLSEYDYDYWGQGTQVTVSS
>L88FB1
(SEQ ID NO: 25)
EVQLVESGGGLVQAGGSLRLSCAASGFTSDDYAIGWFRQAPGKE
REGVSYISAEDGSTYNADSVKGRFTISSDNAKNTVYLQMNSLKP
EDTGVYYCAAALLGASVRWLSEYDYDYWGQGTQVTVSS
>L88FA2
(SEQ ID NO: 26)
EVQLVESGGGLVQAGGSLRLSCAASGFTSDDYAIGWFRQAPGKE
REGVSYISNEDGSTYVADSVKGRFTISSDNAKNTVYLQMNSLKP
EDTAAYYCAAAILGNSVRWLSEYDYDYWGQGTQVTVSS
>L88B10B
(SEQ ID NO: 27)
EVQLVESGGOLVQAGGSLRLSCACSGFTSDDYAIGWFRQAPGKE
REGVSYISREDGSRHYADSAKGRFTISSDYAKNTVYLQMNSLEP
EDTAVYYCAAALLGDYVLWLPEYEYKYWGQGTQVTVSS
>L81H9
(SEQ ID NO: 28)
EVQLVESGGGLVQAGGSLRLSCVCSGFSSDEYAIGWFRQAPGKE
REGVSYISREDGSRHYADSVKGRFTISTDYARNTVYLQMDSLKP
EDTGVYYCAAALLGDYVLWLPEYPYNYWGQGTQVTVSS
>L88B12G
(SEQ ID NO: 29)
EVQLVESGGGLVQAGGSLRLSCACSGFTSDDYAIGWFRQAPGKE
REGVSYISREDGGRYYADSVKGRFTISSDNAKNTVYLQMNSLKP
EDTAVYYCAAALLGDYVRWLPEYEYNYWGQGTQVTVSS
>L81C9
(SEQ ID NO: 30)
EVQLVESGGGLVQAGGSLRLSCAASGFTLDDYAIGWFRQAPGKE
REKVSFMFTSDGSGWYADSVKGRFTISSNNAKNTVYLEMNRLKP
EDTAVYYCAAAYAGELVLWLPDYDYNYWGQGTQVTVSS
L91B5 family:
>L94C2
(SEQ ID NO: 31)
EVQLVESGGGLVQAGGSLRLSCAVSGGTFDDYAIGWFRQAPGKE
REGVSYIGCNDGSTYNAGSVKGRFTISCDYAKNTVYLQMNNLNS
EDTAVYYCAAAAQWATIRWIQEYDYDYWGQGTQVTVSS
>L923A4
(SEQ ID NO: 32)
EVQLVESGGGLVQAGGSLRLSCAVSGGTFDDYAIGWFRQAPGKE
REGVSYIGCNDGSTYNAGSVKGRFTISCDYAKNTVYLQMNNLKP
EDTAVYYCAAAAQWATIRWIQEYDYDYWGQGTQVTVSS
>L911F4C
(SEQ ID NO: 33)
EVQLVESGGGLVQAGGSLRLSCAVSGGTFDDYAIGWFRQAPGKE
REGVSYIGCNDGSTYYANSVKGRFTISCDYAKNTVYLQMNNLKP
EDTAVYYCAAAAQWATIRWIQEYDYDYWGQGTQVTVSS
>L932G9
(SEQ ID NO: 34)
EVQLVESGGGLVQAGGSLRLSCAVSGGTFDDYAIGWFRQAPGKE
REGVSYIGCNDGSTYHADSVKGRFTISCDYAKNTVYLQMNNLKP
EDTAVYYCAAAAQWATIRWIQEYYYDYWGQGTQVTVSS
>L932E10
(SEQ ID NO: 35)
EVQLVESGGGLVQAGGSLRLSCAVSGGTFDDYAIGWFPQAPGKE
REGVSYIGCNDGSTYYADSVKGRFTISCDYPKNTVYLQMNHLKP
EDTAAYYCAAAAQWATIRWIQQYHYDYWGQGTQVTVSS
>L94H1
(SEQ ID NO: 36)
EVQLVESGGGLVQAGGSLRLSCAVSGGTFDDYAIAWFRQAPGKE
REGVSYIGCNDGSTYYAGSVKGRFTISCDNAKNTVYLQMNSLKP
DDTAVYYCAAAAQWATIRWIQEYDYDYWGQGTQVTVSS
>L91B5
(SEQ ID NO: 37)
EVQLVESGGGLVQPGGSLRLSCAASGATFDDYAIGWFRQAPGKE
REGVSYIGCNDGATYYAGSVKGRFTISCDYAKNTVYLQMNSLKP
EDTAVYYCAAAAQWATIRWIHEYDYNIWGQGTQVTVSS
>L93H6
(SEQ ID NO: 38)
EVQLVESGGGLVQPGGSLRLSCAASGATFDDYAIGWFRQAPGKE
REGVSYIGCNDGATYYSGSVKGRFTISCDYAKNTVYLQMNSLKP
EDTAVYYCAAAAQWATIRWIHEYDYNIWGQGTQVTVSS
>L94E3
(SEQ ID NO: 39)
EVQLVESGGGLVQAGGSLRLSCAVSGATFDDYAIGWFRQAPGKE
REGVSYIGCNDGSTYYAGSVKGRFTISCDYAKNTVYLQMNSLKP
EDTAVYYCAAAAQWATIRWIQEYDYDTWGQGTQVTVSS
>L94H2
(SEQ ID NO: 40)
EVQLVESGGGLVQAGGSLRLSCAVSGATFDDYAIGWFRQAPGKE
REGVSYIGCNDGSIYYAGSVKGRFTISCDYAKNTVYLQMNSLKP
EDTAVYYCAAAAQWATIRWIQEYDYDTWGQGSQVTVSS
>L91H9
(SEQ ID NO: 41)
EVQLVESGGGLVQAGGSLRLSCAVSGATFDDYAIGWFRQAPGKE
REGVSYIGCNDGSTYYAGSVKGRFTISCDYAKNTVYLQMNSLKP
EDTAVYYCAAAAQWATIRWIHEYDYDTWGQGTQVITSS
>L94D12
(SEQ ID NO: 42)
EVQLVESGGGLVQAGGSLRLSCAAFGFTFDDYAIGWFRQAPGKE
REGVSYIGCNDYSTYYSDSVKGRFTISCDYAKNTVYLQMNNLKP
EDTAVYYCAAAAQWATIRWIHEYDYNYWGQGTQVTVSS
>L93D4
(SEQ ID NO: 43)
EVQLVESGGGLVQAGGSLRLSCAASGFTFDDYAIGWFRQAPGKE
REGVSYIGCNDGSTYIADSVKGRFTISCDYAENTVYLQMDSLKS
EDTAVYYCAAAAQWATIRWIQEYDYDYWGQGTQVTVSS
>L9Bp5
(SEQ ID NO: 44)
EVQLVESGGGLVQTGGSLRLSCVASGGTLDDYAIGWFRQAPGKE
REGVSYIGSNDGSTYYAGAVKGRFTISSDHAKNTVYLQMNSLKP
EDTAVYYCAAAAQWGTIRWIHEYDYEYWGQGTQVTVSS
>L91E1
(SEQ ID NO: 45)
EVQLVESGGGLVQAGGSLRLSCAASGGTLYDYAIGWFRQAPGKE
REGVAYLGASDGATYYADSVKGRFTISSDNAKNTVYLQMNSLKP
EDTAVYYCAAAAQWATIRWIQEYDYDTWGQGTQVTVSS
L94D4 family:
>L94A12
(SEQ ID NO: 46)
EVQLVESGGGLVQAGGSLRLSCAASGFTFDDYGIAWFRQAPGKE
REGLACITRSGYTTYYLDSVRGRFTISSDNAKNTVYLQMNSLNP
EDTAVYYCAADEPLVVWNCNGDFGSWGQGTQVTVSS
>L94D4
(SEQ ID NO: 47)
EVQLVESGGGLVQAGGSLRLSCATSGFTFDDYGIAWFRQAPGKE
REGLACITRSGYTTYYLDSVKGRFTISSDNAKNTVYLQMNSLNP
EDTGVYYCAADEPLIVWNCNGDFGSWGQGTQVTVSS
L93E3 family:
>L94H5
(SEQ ID NO: 48)
EVQLVESGGGLVQPGGSLRLSCETSRHTLDHYAIGWFRQAPGKE
NEGVACISTSTYYADSVKGRFSISRDNAKNTVYLQMNSLKPEDT
AVYYCATTGSGYCTLRAFASWGQGTQVTVSS
>L9Bf11
(SEQ ID NO: 49)
EVQLVESGGGLVQPGGSLRLSCETSRHTLDHYAIGWFRQAPGKE
NEGVACISTSTYYADSVKGRFSISRDNVKNTVYLQMNSLKPEDT
AVYYCATTGSGYCTLRAFASWGQGTQVTVSS
>L933D10
(SEQ ID NO: 50)
AISSGDGFGRTLGGSLRLSCETSRHTLDHYAIGWFRQAPGKDNE
GVACITTSTYYADSVKGPFSISRDNAKNTVYLQMNSLKPEDTAV
YYCATTGSGYCTLRAFASWGQGTQVIVSS
>L93C3
(SEQ ID NO: 51)
RWQLVESGGGLVQPGGSLRLSCETSRHTLDHYAIGWFRQAPGKG
GEGVACISTNTYYADSVKGRFSISRDNAKNTVYLQMNDLKPEDT
AVYHCATTGSGYCTLRAFASWGQGTQVTVSS
>L9Cl22
(SEQ ID NO: 52)
EVQLVESGGGLVQPGGSLRLSCETSRHTLDHYAIGWFRQAPGKE
GEGVACISTNTYYADSVKGRFSISRDNAKNTVYLQMNDLKPEDT
AVYHCATTGSGYCTLRAFASWGQGTQVTVSS
>L922E2
(SEQ ID NO: 53)
EVQLVESGGGLVQPGGSLRLSCETSRHTLDHYAIAWFRQAPGKE
GEGIACVSTSTYYADSVKGRFSISRGNAKNTVYLQMNDLKPEDT
AVYHCATTGSGYCTLRAFASWGQGTQVIVSS
>L94E4
(SEQ ID NO: 54)
EVQLVESGGGLVQPGGSLRLSCETSRHTLDHYAIGWFRQAPGKE
GEGVACISTSTYYADSVKGRESISRDNAKNTVYLQMNSLKPEDT
AVYYCATTGSGLCTLRAFASWGQGTQVITSS
>L93E3
(SEQ ID NO: 55)
EVQLVESGGGLVQPGGSLRLSCAASQFTLESYAIGWFRQAPGKD
SEGVACISSSTYYADSVKGRFTISRDNAKNTVYLQMESLKPEDT
AVYHCATSGAGSYCTLRAFGSWGQGTQVTVSS
>L9Bm16
(SEQ ID NO: 56)
EVQLVESGGGLVQPGGSLRLSCAASQFTLESYAIGWFRQAPGKD
SEGVACISSSTYYADSVKGRFTISRDNAKNTVYLQMESLKPEDT
AVYHCATSGAGSYCTLRAFGSWGQGTQVTVSS
>L94E5
(SEQ ID NO: 57)
EVQLVESGGGLVQPGGSLRLSCAASQSTLESYAIGWFRQAPGKD
SEGVACISSSTYYADSVKGRFTISRDNAKNTVYLQMESLKPEDT
AVYHCATSGAGSYCTLRAFGSWGQGTQVTVSS
L91F10 family
>L932A9
(SEQ ID NO: 58)
EVQLVESGGGLVQPGGSLRLSCAASGFTLDGYAIGWFRQAPGKE
REAISCITTRGTIYYADSVKGRVTISRDNAKNTVYLQMTSLKPE
DTAVYLCAADGFDTPCVAGTDWGYDYEGQGTQVTVSS
>L93F2
(SEQ ID NO: 59)
EVQLVESGGSLVQPGGSLRLSCAASGFTLDGYAIGWERQAPGKE
REAISCITTRGTIYYADSVKGRVTISRDNAKNTVYLQMTSLKPE
DTAVYLCAADGFDTPCVAGTDWGYDYEGQGTQVTVSS
>L95B8A
(SEQ ID NO: 60)
EVQLVESGGGLVQPGGSLRLSCAASGFTLDGYAIGWFRQAPGKE
REAISCITTRGTIYYADSVKGRVTISRDNAKNTVYLQMNSLKPE
DTAVYLCAADGFDTPCVAGTDWGYDYEGQGTQVTVSS
>L91C2
(SEQ ID NO: 61)
EVQLVESGGGLVQPGGSLRLSCAASGFTLDGYAIGWFRQAPGKE
REAISCITTAGTIYYADSVKGRVTISRDNAKNTVYLQMNSLKPE
DTAVYLCAADGFDTPCVAGTDWGYDYEGQGTQVTVSS
>L92C7
(SEQ ID NO: 62)
EVQLVESGGGLVQPGGSLRLSCAASGFTLDGYAIGWFRQTPGKE
REAISCITTAGTIYYADSVKGRVTISRDNAKNTVYLQMNSLKPE
DTAVYLCAADGFDTPCVAGTDWGYDYEGQGTQVTVSS
>L92E6
(SEQ ID NO: 63)
EVQLVESGGGLVQPGGSLRLSCAASGFTLDGYAIGWFRQAPGKE
REAISCITTAGTIYYADSVKGRVTISRDNAKNTVYLQMNSLKPE
DTAVYLCAVDGFDTPCVAGTDWGYDYEGQGTQVTVSS
>L91G2
(SEQ ID NO: 64)
EVQLVESGGGLVQPGGSLRLSCAASGFTLDGYAIGWFRQAPGKE
REAISCITTAGTIYYADSVKGRVTISRDNTKNMVYLQMNSLKPE
DTAVYLCAADGFDTPCVAGTDWGYDYEGQGTQVTVSS
>L911B1G
(SEQ ID NO: 65)
EVQLVESGGGLVQPGGSLRLSCVASGFTLDGYAIGWFRQAPGKE
REAISCITTAGTIYYADSVKGRVTISRDNAKNTVYLQMNSLKPE
DTAVYLCAADGFDTPCVAGTDWGYDYEGQGTQVTVSS
>L95B12E
(SEQ ID NO: 66)
EVQLVESGGGLVQLGGSLRLSCAASGFTLDGYAIGWFRQAPGKE
REAISCITTAGTIYYADSVKGRVTISRDNAKNTVYLQMNSLKPE
DTAVYLCAADGFDTPCVAGTDWGYDYEGQGTQVTVSS
>L93C6
(SEQ ID NO: 67)
EVQLVESGGGLVQPGGSLRLSCAASGFTLDGYAIGWFRQAPGKE
REAISCITTAGTIYYADSVKGRVTISRDNAKNTVYLQMNSLKPE
DTAVYLCAADGFDTPCVAGTDWGYDYGGQGTQVTVSS
>L95B12A
(SEQ ID NO: 68)
EVQLVESGGGLVQAGGSLRLSCAASGFTLDGYAIGWFRQAPGKE
REAISCITTAGTINYADSVKGRVTISRDNAKNTVYLQMNSLKPE
DTAVYLCAADGFDTPCVAGTDWGYDYEGQGTQVTVSS
>L91F10
(SEQ ID NO: 69)
EVQLVESGGGLVQPGGSLTLSCVASGFTLDGYAIGWFRQAPGKE
VESVSCITGRHGTIYYADSVMGRFTISRDNAKSTVYLQMNSLKP
EDTAVYYCAADGFDTPCVAGTDWQYDYWGQGTQVTVSS
>L93B3
(SEQ ID NO: 70)
EVQLVESGGGLVQPGGSLTLSCVASGFTLDGYAIGWFRQAPGKE
VESVSCITGRHGTIYYADSVMGRFTISRDNAKNTVYLQMNSLKP
EDTAVYYCAADGFDTPCVAGTDWQYDYWGQGTQVTVSS
>L911B12B
(SEQ ID NO: 71)
EVQLVESGGGLVQPGGSLTLSCVASGFTLDGYAIGWFRQAPGKE
VEPVSCITGRHGTIYYADSVMGRFTISRDNARNTVYLQMNSLKP
EDTAVYYCAADGFDTPCVAGTDWQYDYWGQGTQVTVSS
L8Cj3 family:
>L833E1
(SEQ ID NO: 72)
EVQLVESGGGLVQAGGSLRLSCIVSESIFSRYAMGWFRQAPGKE
REFVAGIGAVTHYGEFVKGRFTISRDSAKNTIYLQMSSLKPEDT
AIYFCARSKNTYISYASNQYDVWGRGTQVTVSS
>L8Cj3
(SEQ ID NO: 73)
EVQLVESGGGLVQAGGFLRLSCELRGSIFNQYAMAWFRQAPGKE
REFVAGMGAVPHYGEFVKGRFTISRDNAKSTVYLQMNSLKPEDT
AIYFCARSKSTYISYNSNGYDYWGRGTQVTVSS
L92E7 family:
>L92E7
(SEQ ID NO: 74)
EVQLVESGGGLVQPGGSLRLSCAASGNIVSIDAAGWFRQAPGKQ
REPVATILTGGATNYADSVKGRFTISRDNAKNTVYLQMSSLKPE
DTAVYYCYAPMIYYGGRYSDYWGQGTQVTVSS
>L911B3D
(SEQ ID NO: 75)
EVQLVESGGGLVQPGGSLRLSCTASGNIVSIDAAGWFRQAPGKQ
REPVATILTGGTTNYADSVKGRFTISRDTAKNIVYLQMNSLKPE
DTAVYYCYAPMIYYGGSYSDYWGQGTQVTVSS
>L98FB10
(SEQ ID NO: 76)
EVQLVESGGGLVQAGGSLRLSCTASINAVSIDAAGWFRQAPGMQ
REPVATILTSGSSTYADSVKGRFTISRDSAKNTVYLQMNSLKPE
DTAVYTCYAPMVYYSGSYSDYWGQGTQVTVSS
>L911F1F
(SEQ ID NO: 77)
EVQLVESGGALVQAGRSLRLSCAASGNAFTIDAAAWYRQAPGKQ
REPVATILSGGTTNYADSVKGRFTISRDNVKNTVYLQMNSLKPE
DTAVYYCYVPMVYYSGRYNDVWGQGTQVTVSS
2E7 family approach:
>L92E7,B5
(SEQ ID NO: 78)
EVQLVESGGGLVQAGGSLRLSCAASGNAFSIDAAAWYR*APGKQ
REPVATILTGGSTNYADSVKGRFTISRDNAKNTVYLQMNSLKPE
DTAVYYCYVPMVYYSGRYSDYWGQGTQVTVSS
>L92E7,C1
(SEQ ID NO: 79)
EVQLVESGGGLVQAGGSLRLSCAASGNAFSIDAAAWYRQAPGKQ
REPVATILTGGSTNYADSVKGRFTISRDNAKNLVYLQMNSLKPE
DTAVYYCYVPMVYYSGRYSDYWGQGTQVTVSS
>L92E7,A4
(SEQ ID NO: 80)
EVQLVESGGGLVQAGGSLRLSCAASGNAFSIDAAAWYRQAPGKQ
REPVATILTGGSTNYADSVKGRFTISRDNAKNTVYLQMNSLKPE
DTAVYYCYVPMVYYSGRYSDYWGQGTQVTVSS
>L92E7,G4
(SEQ ID NO: 81)
EVQLVESGGGLVQPGGSLRLSCAASGNIVSIDAAGWFRQAPGKQ
REPVATILTGGATNYADSVKGRFTISRDNAKNTVYLQMNSLKPE
DTAVYYCYAPMIYYGGRYFDYWGQGT*VTVSS
>L92E7,C3
(SEQ ID NO: 82)
EVQLVESGGGSLQAGGSLRLSCAASGNIVSIDAAGWFRQAPGKQ
REPVATILTGGATNYADSVKGRFTISRDNAKNTVYLQMNSLKPE
DTAVYYCYAPMIYYGGRYSDYWGQGTQVTVSS
>L92E7,D3
(SEQ ID NO: 83)
EVQLVESGGGLVQPGGSLRLSCAASGNIVSIDAAGWFRQAPGKQ
REPVATILTGGATNYADSVKGRFTISRDNAENTVYLQMNSLKPE
DTAVYYCYAPMIYYGGRYSDYWGQGTQVTVSS
>L92E7,E1
(SEQ ID NO: 84)
EVQLVESGGGLVQPGGSLRLSCAASGNIVSIDAAGWFRQAPGKQ
REPVATILTGGAANYADSVKGRFTISRDNAENTVYLQMNSLKPE
DTAVYYCYAPMIYYGGRYSDYWGQGTQVTVSS
In vitro maturation:
>L92E7,A1
(SEQ ID NO: 85)
EVQLVESGGGLVQPGGSLRLSCAASGNIASIDAAGWFRQAPGKQ
REPVATILTGGATNYADSVKGRFTISRDNAKNTVYLQMNSLKPE
DTAVYYCYAPMIYYGGRYSDYWGQGTQVTVSS
>L92E7,A2
(SEQ ID NO: 86)
EVQLVESGGGLVQPGGSLRLSCAASGNIVSIDAAGWFRQAPGKQ
REPVATILTGGATNYADSVKGRFTISRDNAKNTVYLQMNSLKPE
DTAVYYCYAPMIAYGGRYSDYWGQGTQVTVSS
>L92E7,A3
(SEQ ID NO: 87)
EVQLVESGGGLVQPGGSLRLSCAASGNIVSIDAAGWFRQAPGKQ
REPVATILTGGATNYADSVKGRFTISRDNAENTVYLQMNSLKPE
DTAVYYCYAPMIYYGGAYSDYWGQGTQVTVSS
>L92E7,A4
(SEQ ID NO: 88)
EVQLVESGGGLVQPGGSLRLSCAASGNIVSIDAAGWFRQAPGKQ
REPVATILTGGATNYADSVKGRFTISRDNAKNTVYLQMNSLKPE
DTAVYYCYAPMIYYGGRASDYWGQGTQVTVSS
>L92E7,A5
(SEQ ID NO: 89)
EVQLVESGGGLVQPGGSLRLSCAASGNIVSIDAAGWFRQAPGKQ
REPVATILTGGATNYADSVKGRFTISRDNAKNTVYLQMNSLKPE
DTAVYYCYAPMIYYGGRYSDAWGQGTQVTVSS
>L91B5,A4
(SEQ ID NO: 90)
EVQLVESGGGLVQPGGSLKLSCAASGAAFDDYAIGWFRQAPGKE
REGVSYIGCYDGAQYYAGSVKGRFTISCDYAKNTVYLQMNSLKP
EDTAVYYCAAAYQWSTIRWVHEYDYNHLGQGTQVTVSS
>L91B5,A7
(SEQ ID NO: 91)
EVQLVESGGGLVQPGGSLKLSCAASGASFHDYAIGWFRQAPGKE
REGVSYIGCDDGAQYYAGSVKGRFTISCDYAKNTVYLQMNSLKP
EDTAVYYCAAAYQWDTIRWVHEYDYSVWGQGTQVTVSS
>L91B5,A12
(SEQ ID NO: 92)
EVQLVESGGGLVQPGGSLKLSCAASGAGFNDYAIGWFRQAPGKE
REGVSYIGCLDGAQYYAGSVKGRFTISCDYAKNTVYLQMNSLKP
EDTAVYYCAAAEQWNTIRWVHEYDYGDWGQGTQVTVSS
>L91B5,B2
(SEQ ID NO: 93)
EVQLVESGGGLVQPGGSLKLSCAASGAAFHDYAIGWFRQAPGKE
REGVSYIGCTDGAQYYAGSVKGRFTISCDYAKNTVYLQMNSLKP
EDTAVYYCAAADQWNTIRWVHEYDYVYWGQGTQVTVSS
>L91B5,D4
(SEQ ID NO: 94)
EVQLVESGGGLVQPGGSLKLSCAASGAPFMDYAIGWFRQAPGKE
REGVSYIGCADGAQYYAGSVKGRFTISCDYAKNTVYLQMNSLKP
EDTAVYYCAAADQWTTIRWVHEYDYHVWGQGTQVTVSS
>L91B5,F4
(SEQ ID NO: 95)
EVQLVESGGGLVQPGGSLRLSCAASGADFDDYAIGWFRQAPGKE
REGVSYIGCDDGAQYYAGSVKGRFTISCDYAKNTVYLQMNSLKP
EDTAVYYCAAANQWTTIRWVHEYDYDIWGQGTQVTVSS
>L91B5,F11
(SEQ ID NO: 96)
EVQLVESGGGLVQPGGSLKLSCAASGANFADYAIGWFRQAPGKE
REGVSYIGCDDGAQYYAGSVKGRFTISCDYAKNTVYLQMNSLKP
EDTAVYYCAAATQWNTIRWVHEYDYVFWGQGTQVTVSS
>L91B5,4A7
(SEQ ID NO: 97)
EVQLVESGGGLVQPGGSLKLSCAASGASFMDYAIGWFRQAPGKE
REGVSYIGCEDGAQYYAGSVKGRFTISCDYAKNTVYLQMNSLKP
EDTAVYYCAAAKQWETIRWVHEYDYHNWGQGTQVTVSS
Other VHH:
>L81H1
(SEQ ID NO: 98)
EVQLVESGGGLVQAGGSLRLSCVLSGGTLSDAAMGWFRQPPGKE
REFVAAISWSAGPTTHYADSVKGRFTISRDDAKNTVYLQMNSLK
PEDTGVYYCAAQFRVGWVHLSNEYDYWGQGTQVTVSS
>L82B1A
(SEQ ID NO: 99)
EVQLVESGGGLVQAGDSLRLSCAASTRTFRNYNLGWFRQAPGKE
REFVAAISWADYRTFYSDSAKGRFTISRDNTKNMLYLQMNSLKP
EDTARYYCGAGYRDVRVDWQPAFWGQGTQVTVSS
>L82B1D
(SEQ ID NO: 100)
EVQLVESGGGLVQAGDSLRLSCTASGSQFSHTDMAWARQPPGKE
REFVAINWSAGNTYYRDSVKGRFTISRDNAQNTVYLQMNSLKPE
DTATYYCAARIGYDVDWNYWGQGTQVTVSS
>L82B2A
(SEQ ID NO: 101)
EVQLVESGGGLVQAGGSLRLSCTASGTTFFMYAMGWFRQAPGKE
REFVAAISRFGDSLHSSDSVKGRFTISRDNTENTVYLQMNMVKP
EDTAVYYCAAKVAYHRSGSRYYTNHTDYDAWGQGTQVTVSS
>L82B3F
(SEQ ID NO: 102)
EVQLVESGGGLVQAGGSLRLSCTVSGQTSTTDAVGWFRQAPGKE
REFVSAISWSDNKLYYEDSVKGRFAISRDNAKDTVYLQMDSLKP
EDTAVYYCAARNRPPDFLTNYFRVLYYREGAYDYWGQGTQVTVS
S
>L82B4A
(SEQ ID NO: 103)
EVQLVESGGGLVQAGGSLRLSCAASGFSLDDYAIGWFRQAPGKE
REGLSCRSGSDGSTYYADSVKGRFTFSSDNAKNTVYLQLNSLKP
EDTAVYYCALITGSSYCLWRRILESEFDVWGQGTQVTVSS
>L82B4E
(SEQ ID NO: 104)
EVQLVESGGGVVQAGGSLRLSCAASGRTFSLYTMGWFRQAPGKE
REFIAHMRRGGYTTYVADSVKDRFTISGDNAEATVYLQMNSLKS
EDTAVYYCAAKLGLLVIGTAPTANDYDYWGQGTQVTVSS
>L82B12A
(SEQ ID NO: 105)
EVQLVESGGDSVQPGGSLRLSCAASGFAYGTLDDYTIGWFRQVP
GKEREGVACIGHNDDTTYYADSVQGRFTISRDNAKNTVYLQMSS
LKPEDTAVYLCASTSRARCARYSYNDRWFFDTWGQGTQVTVSS
>L88B2A
(SEQ ID NO: 106)
EVQLVESGGGLVQAGGSLRLSCVASGDIQFSWSSMAWFRQAPGK
EREFVATISRSGSDVDYADSAKGRFTISRDNAKKMLYLQMNSLK
PEDTATYYCAAARGLTAYEFQFWGQGTQVTVSS
>L88FA3
(SEQ ID NO: 107)
EVQLVESGGGLVQAGDSLIMSCTASTPTFSTLAMAWFRQAPGKE
REFVAGIVDGMDNYDYSVEGRFTISRDNRENTLYLQMDSLRPED
TAVYYCAARRRYYSSKYEYWGQGTQVTVSS
>L88FA5
(SEQ ID NO: 108)
EVQLVESGGGLVQAGGSLRLSCAASFSSDNMGWFRQAPGKEREF
IAAINWNGGTIVYADSVKGRFTISRDNAICNTVYLQMNSLKPED
TAVYYCAADSPGVVLSWDYWGQGTQVTVSS
>L89B1D
(SEQ ID NO: 109)
EVQLVESGGGSVQAGDSLKLSCTASTPTFSNLAMAWFRQAPGRE
REFVSGIVGDDITNYADFAEGRFTISRDNSKNTLYLQMDSLRPE
DTAVYVCAARRRYYSSRYEYWGQGTQVTVSS
>L89B2C
(SEQ ID NO: 110)
EVQLVESGGGLVQAGGSLRISCEYSGRTFSTHRMAWFRQAPGKD
REFVASLKWSDGETTYADSVKGRFTISRENAENSLYLQMNNLEP
GDTAVYFCAAGTWWPPSYDYWGQGTQVTVSS
>L89B6B
(SEQ ID NO: 111)
EVQLVESGGGLVQTGGSLRLSCTASGQTSIIDAVGWFRQAPGKE
REFVSAISWSDNNIYYEDSVKGRFTISRDNAKDTVYLQMDSLKP
EDTAVYYCAARNRPPDFLTNYFRVLYYREGAYDYWGQGTQVTVS
S
>L89B12D
(SEQ ID NO: 112)
EVQLVESGGGLVQSGDSLRLSCTISGIFFDLYSMAWFRQAPGKE
REFVAAISSVGGMTDYADSVKGRFTISRDNARQTVYLQMNSLKP
EDTAVYSCAARAHREIVRSYGYNYWGQGTQVTVSS
>L832G6
(SEQ ID NO: 113)
EVQLVESGGDLVQAGDSLRLSCVVSGSIRDNSVIGWFRQVPGKE
RKLFTYYYWSGTSTYFVEAVKGRFTISRDIKSKNTVYLQMNNLK
EIEDTALYYCATTSEGIMIDISGQGTQVSFLS
>L833H1
(SEQ ID NO: 114)
EVQLVESGGGLVQAGDSLRLSCAVSGSISGIKTIGWFRQFPGRE
RKLFGYYYWNGASTYLVDSVICGRFTISRDNAENTVYLQMNNLK
PEDTAIYYCGAETEGHYDFEFWGQGTQVTVSS
>L833H3
(SEQ ID NO: 115)
EVQLVESGGGLVQAGGSLRLSCVLSGGTFSDAAMGWFRQPPGKE
REFNTAAISWSAGPTTHYADSVKGRFTISRDDAKNTVYLQMNSL
KPEDTGVYYCAAQFRVGWVHLSNEYDYWGQGTQVTVSS
>L8Cb15
(SEQ ID NO: 116)
EVQLVESGGGAVQPGDSLRLACTASGVTLDASRVTLDVYSIGWF
RQAPGNEREAVSCISSSDGSTYYADSVKGRFTISRDDAKNTVYL
QMNSLKPEDTAVYYCASDWSCEIVLYATADYWGQGTQVTVSS
>L8Fe2
(SEQ ID NO: 117)
EVQLVESGGGLVQAGDSLRLSCTLSGGTYGFTEYRIGWFRQAPG
LEREFAASIAWHDDTYYADSVRGRFLISRDNAKNTVQLQMNSLK
PEDTAVYYCAAGLPGSPRRYDYWGQGTQVTVSS
>L8Fg12
(SEQ ID NO: 118)
EVQLVESGGGLVQAGGSLRLSCAAAVSATRINAFGWYRQAPGKQ
RELVATILSDGNTNYADSVKGRFTISRDNTRNTVYLQMNGLKPE
DTAVYVCNAELYYSDYLPTQTAWGQGTQVTVSS
>L8Fj19
(SEQ ID NO: 119)
EVQLVESGGGLVQAGGSWLSCAASGGTFRSYSMGWFRQAPGKER
EFVGRITWTGSTSYADFVKGRFTISRDNAENMMYLQMNSLKPED
TAIYYCARDRTGLRYLTAQAMRDTNEYEYWGQGTQVTVSS
>L8Fo17
(SEQ ID NO: 120)
EVQLVESGGGLVQAGGSLRLSCAASGRSFRVYGMAWFRQAPGKE
REFVGRITWTASTSYADSVKGRFTISRDSAMNVIYLQMNSLKPE
DTAVYYCATDRVSARYLTAQAMRDTDEYDYWGQGTQVTVSS
>L8Fp6
(SEQ ID NO: 121)
EVQLVESGGGLVQAGGSLRLSCAASGRTINTYATGWFRQAPGKE
REFVARISWGADSTNYADSVKGRFTISKDNAKNTVYLQMNSLKP
EDTAVYYCAADKPYRALNLERMHDSSEYDVWGQGTQVTVSS
>L8Hi20
(SEQ ID NO: 122)
EVQLVESGGGLVQTGGSLTLSCATSEGTFSAYTMAWFRQAPGKE
REFVGAIDWRFGNTYYADSVKGRFTISRDNARNTGYLQMNSLKP
EDTAVYFCAADRYRSIPYPPRRDSVYEYWGQGTQVTVSS
>L91A1
(SEQ ID NO: 123)
EVQLVESGGGLVQPGGSLRLSCAVSGFPLGYYAIGWFRQAPGKE
REEVSCIVTSDGSTYYTDSVKGRFTISRDAAKNTVYLQMNNLNP
EDTAVYYCAAGGTLVAVTDSCFIDYVMDYWGKGTLVTVSS
>L91E2
(SEQ ID NO: 124)
EVQLVESGGGLVQAGGSLRLSCTTSGSIFSLSGMGWFRQAPGKQ
REPVAVITSGGATNYAPYAKGRFTISRDNAKNTVYLQMNSLKPG
DTAVYYCALDSPDRSLGAPPFDYWGQGTQVTVSS
>L92A11
(SEQ ID NO: 125)
EVQLVESGGGLVQPGGSLRLSCAVSGFPLGYYAIGWFRQAPGKE
REEVSCIVTSDGSTYYTDSVKGRFTISRDNAKNTVYLQMNNLNP
EDTAVYYCAAGGTIVAVTDSCFIDYVMDYWGKGTLVTVSS
>L92D4
(SEQ ID NO: 126)
EVQLVESGGGSVQAGASLRLSCAASGRTFSGIDMAWYRQAPGNG
RELVAVITSGGSTKYVDSVKGRFTIFRDNAKNTVALQMNSLKPE
DTAVYYCAAVEESPFYSGTYYPKPGDYWGQGTQVTVSS
>L93B4
(SEQ ID NO: 127)
EVQLVESGGGLVQPGGSLRLSCAVSGFPLGYYAIGWFRQAPGKE
REEVSCIVTSDGSTYYTDSVKGRFTISRDAAKNTVYLQMNNLNP
EDTAVYYCAAGGTLVAVTDCFIDYVMDYWGKGTLVTVSS
>L91H3
(SEQ ID NO: 128)
EVQLVESGGGLVQPGGSLRLSCAASGSILDDANAMGWYRQTPGT
ERALVALITDSGATRYADSVKGRFTISRDNAKNTATLQMNSLKP
EDTAVYYCNFREFGGWGTNIDHWGQGTQVTVSS
>L93D3
(SEQ ID NO: 129)
EVQLVESGGGLVQPGGSLRLSCAASGFGLDYYAIGWFRQAPGKE
REGVSCIDSSDGSTYYVDSAKGRFTISRDNAKNTVYLQMNNLKP
EDTAVYYCAAHALICGSYWSNGVVFDTWGQGTQVTVSS
>L93D9
(SEQ ID NO: 130)
EVQLVESGGGLVQAGGSLRLSCATSGFRFDDYDIGWFRQAPGKE
REGVSCISRGDGMTYYADFVKGRFTISSDSAKNTVYLQMNSLKP
EDTAVYYCAADPGRSVGWVRAWSSTAQTMGVPSPADFASRGQGT
QVTVSS
>L93E6
(SEQ ID NO: 131)
EVQLVESGGGLVQPGGSLRLSCAASGFTLDNYAIGWFRQVPGKE
REGVSCISSSDGKTYYADSVKGRFTISRDNAKNTVYLQMNSLKP
EDTAVYYCARETRCLGSRWMGGGQGTQVTVSS
>L93F2
(SEQ ID NO: 132)
EVQLVESGGGLVQPGGSLRLSCAVSGFPLGYYAIGWFRQAPGKE
REEVSCIVTSDGSTYYTNSVKGRFTISRDNAKNTVYLQMNNLNP
EDTAVYYCAAGGTIVAVTDSCFIDYVMDYWGKGTLVTVSS
>L93F12
(SEQ ID NO: 133)
EVQLVESGGSLVQPGGSLRLSCAASGFTLDGYAIGWFRQAPGKE
REAISCITTRGTIYYADSVKGRVTISRDNAKNTVYLQMTSLKPE
DTAVYLCAADGFDTPCVAGTDWGYDYEGQGTQVTVSS
>L93H9
(SEQ ID NO: 134)
EVQLVESGGGLVQAGGSLRLSCRASGNTGNVNAMGWYRQAPGKQ
RELVAAVSGGGKTNYADFVKGRFTISIDNAKIIVYLQMNNLKPE
DTAVYYCNAGVMGTSGSYYLGEDNWGQGTQVTVSS
>L94H7
(SEQ ID NO: 135)
EVQLVESGGGLVQPGGSLRLSCTASGFQLVYHDLGWFRQVPGKE
REGISCISSTDGSTYYTDSVKGRFTISRDNAKNIVYLQMNSLKS
EDTAVYYCAADWRNVCRLPLGTGFGSWGQGTQVTVSS
>L95B10D
(SEQ ID NO: 136)
EVQLVESGGGLVQAGGSLTLSCEASGTIFSINGQGWFRQAPGKQ
REPVAFITSGGMTNYADHVKGRFPISRDNAKNTVYLQMNSLKPE
DTAVYYCAAEDSPPNYRCSGEWCFDYWGQGTQVTVSS
>L911B1E
(SEQ ID NO: 137)
EVQLVESGGGLVQPGGSLRLSCAASGNIFGENTMNWYRQAPGKQ
RELVAGITNLQSAYYPDGVKYADSVKGRFTISRDNAKNAVYLQM
NSLKPEDTAVYYCNIFEHRFPPATYWGQGTQVTVSS
>L911B2E
(SEQ ID NO: 138)
EVQLVESGGGLVQPGGSLRLSCAASGPLFSISTMGWYRQAPGEQ
RELVASISSDGDTNYSDSVKDRFTISRDNAENTVYLQMNSLKPE
DTAVYFCYARKWILDTYWGQGTQVTVSS
>L911B9A
(SEQ ID NO: 139)
EVQLVESGGGLVQAGGSLRLSCTASGVTIDAVGWYRQAPGKQRE
PVATITSGGSTNYADSVKGRFTISRDDAKNTVYLQMNSLKPEDT
AVYYCFRPGGPYSDSWGQGTQVTVSS
>L911B11E
(SEQ ID NO: 140)
EVQLVESGGGLVQPGGSLRLSCAASGSIFSMTGMGWYRQPPGKQ
RELVAAITTDGSTHYADSVKERFTISGDNAKKTLYLQMNSLKPE
DTAVYYCYAPMIYDRGSYRDYWGQGTQVTVSS
>L911F1B
(SEQ ID NO: 141)
EVQLVESGGGLVQPGGSLRLSCAASGNILGENTMNWYRQAPGKE
RELVAGVTNLRSTYYPDGVKYADSVKGRFTVSRDNAKNAVYLQM
NSLKPEDTAVYYCNLFEHRFPPATYWGQGTQVTVSS
>L911F12B
(SEQ ID NO: 142)
EVQLVESGGALVKAGGSLRLSCAVSGTSVSDNGMGWGRQAPGKQ
RELVAVLTSGGATNYAPSVKGRFTISRDNAKNAVYLQMNNLKPE
DTAVYYCAADTAARSLYSDPFDVWGQGTQVTVSS
>L922B2
(SEQ ID NO: 143)
EVQLVESGGGLVQAGGSLRLSCRASGNTGNVNAMGWYRQAPGKQ
RELVAAVSGGGKTNYADSVKGRFTISIDNAKIIAYLQMNSLKPE
DTAVYYCNAGVMGTSGSYYPGDDNWGQGTQVTVSS
>L922B4
(SEQ ID NO: 144)
EVQLVESGGGLVQAGGSLRLSCRASGNTGNVNAMGWYRQAPGKQ
RELVAAVSGRGKTNYADSVKGRFTISIDNAKIIVYLQMNNLKPE
DTAVYYCNAGVMGTSGSYYLGEDNWGQGTQVTVSS
>L922E1
(SEQ ID NO: 145)
EVQLVESGGGLVQAGGSLRLSCAASGNTGNVNAMGWYRQAPGKQ
RELVAGISSRGTTNYADSVKGRFTISLDSAKITAHLQMNRLKPE
DTAVYYCNAGVMGSSGSYYPGEENWGQGTQVTVSS
>L923A1
(SEQ ID NO: 146)
EVQLVESGGGLVQAGGSLRLSCRASGNTGNVNAMGWYRQAPGKQ
RELVAAVSGGGKTNYADSVKGRFTISIDNAKIIVYLQMNNLKPE
DTAVYYCNAGVMGTSGSYYLGEDNWGQGTQVTVSS
>L932E8
(SEQ ID NO: 147)
EVQLVESGGGLVQAGGSLRLSCETSGNTVDDYAMGWFRQAPGKE
GEVVACISTSGYTDYADSGKGRFSISIDSNMTLHLHMKRLKHAD
ADLCYSTASVMCSSGTYYPGEENWREGTPVTVSS
>L9Ab16
(SEQ ID NO: 148)
EVQLVESGGGLVQAGGSLRLSCAASGIIFGSKGMGWYRQAPGKQ
RELVATVTSGGDIKYADSVKGRFSISRDNAKNTVYLQMNSLKPE
DTAVYYCAALFPLYGTGELTPLDDYWGQGTQVTVSS
>L9Ab19
(SEQ ID NO: 149)
EVQLVESGGGLVQAGGSLRLSCVASGIIFSDSGMGWFRQAPGKQ
REPVAVITRGGSTNYADSVKGRFTISRDNAKNTVYLQMNNLKPE
DTAVYAADTFDRHLQAPPFDYWGQGTQVTVSS
>L9Ad3
(SEQ ID NO: 150)
EVQLVESGGGLVQAGGSLRLSCTASESSFSLTSMGWYRQAPGKQ
REAVASIDGDGTTSYADSVKGRFTISRDNAKNTVNLQMNSLKPE
DTAVYFCFARKWILDIYWGQGTQVTVSS
>L9Ad13
(SEQ ID NO: 151)
EVQLVESGGGLVQPGGSLRLSCAAPKIFSTAPMNWFRQAPGKQR
ELVAGISSGGSTMYADSVKGRFTISRDSAKNTLDLQMNSLKPED
TAVYYCNAEWAPLGPRSRVDYWGQGTQVTVSS
>L9Ad14
(SEQ ID NO: 152)
EVQLVESGGGWVQPGGSLGLSCTAGRSAFSINDMGWYRQPPGKQ
REVVAIITFDGSANYADSVKGRFTISRRNYNNTVYLQMNNLKPE
DTAVYYCNADAAASPRGGYNEYWGQGTQVTVSS
>L9Aj2
(SEQ ID NO: 153)
EVQLVESGGGLVQPGGSLRLSCAASGFTLDTYAIGWFRQAPGKE
REGVSCISKIDEQRYYLDSVKGRFTISRDNAKNTVYLQMNSLKP
EDTAVYYCATPRSQYYCHNGAIGYDRWGQGTQVTVSS
>L9An7
(SEQ ID NO: 154)
EVQLVESGGGLVQSGGSLRLSCAASGFTLDYYTVGWFRQAPGKE
REGVSCINSSDGRTLYADSVKGRFTISRDNAKNTVYLQMNSLKP
EDTAVYYCAAEKPGLTSYYYPCPPPGEYDYSGQGTQVTVSS
>L9Ao15
(SEQ ID NO: 155)
EVQLVESGGGLVQTGGSLRLSCAASGGTLSSYAMGWFRQAPGKE
REFVAAITWSGTRTLYADSVKGRFTISRDNANNTVYLQMNSLEP
EDTAVYYCAAHRHPYALVVDHKAFDYWGQGTQVTVSS
>L9Ap11
(SEQ ID NO: 156)
EVQLVESGGGSVQAGGSLRLSCAASGSIFNINAMGWYRQARGKG
RELVAVVTNGGTTTYTGSVKGRFTISRDNAKNTVYLQMNSLKLE
DTAVYYCAGVEESPFHSGTYYPLPGDYWGQGTQVTVSS
>L9Bb3
(SEQ ID NO: 157)
EVQLVESGGGLVQAGGSLRLSCAASGRTFGTYAMAWFRQAAGKE
REFVGAISSWGGGRTYYPPSVQGRFTISRENAKNTVYLQLSSLK
PEDTAVYYCAAKRDNCSAFGCYAGPYDYWGQGTQVTVSS
>L9Bc6
(SEQ ID NO: 158)
EVQLVESGGGLVQAGGSLRLSCAASGFTFDDYAIGWFRQAPGKE
REGVLCFTRSRGSTDGSTYYADSVKGRFTISSDNAKNTVYLQMN
SLKPEDTAVYYCATDHYLRVGLKCRDYEYDYWGQGTQVTVSS
>L9Bd8
(SEQ ID NO: 159)
EVQLVESGGGLVQPGGSLRLSCIASGFTLDYYAMGWFRQAPGKE
REGVACISSTDRSTYYPDSVKGRFTISRDNAENTVYLQMNSLKP
EDTAVYSCAVGWNPDCENGMDYWGKGTLVTVSS
>L9Bd9
(SEQ ID NO: 160)
EVQLVESGGGLVQTGGSLRLSCVASGNIYSLTSTGWYRQAPGQQ
REWIATIRGDDSTEYANSVKGRFTISRENALKTVYLQMNSLKPE
DTAIYYCAARTLPLGQPNYWGQGTQVTVSS
>L9Be11
(SEQ ID NO: 161)
EVQLVESGGGLVQSGGSLRLSGAASGFTLDSYAIGWFRQAPGKE
REGVSCISNSDGRTYYADSVKGRFTISRDSAENTVYLQMNSLKP
EDTAVYLCATEGHCHGTNWTRPHSVSQGTQVTVSS
>L9Bf19
(SEQ ID NO: 162)
EVQLVESGGGLVQPGGSLRLSCTASGRSISSSAMGWFRQAPGKE
REFVASVHWTDVSTWYADSVKGRFTISRDNAKNTVYLQMNSVKP
EDTAVYHCAASRAFSPDSWRRYSDSDSYESWGQGTQVTVSS
>L9Bj13
(SEQ ID NO: 163)
EVQLVESGGGLVQAGGSLRLSCAASGSISSLNTMAWYRQTPGQQ
REWVARITGAGSTTYADSVKGRFTISRDDALYITYLQMTSLKPE
DTAVYYCAARTLPLGADNWWGQGTQVTVSS
>L9Bm10
(SEQ ID NO: 164)
EVQLVESGGGLVQPGGSLRLSCAASGFTLDDYTTGWFRQAPGKE
REGVSCISSSDGGTWYADSVKGRFTISRDNAKNTVYLQMNSLKP
EDAAVYYCAVDSTAGSSCPGPDFYAMDYWGKGTLVTVSS
>L9Bp16
(SEQ ID NO: 165)
EVQLVESGGGLVQAGESLRLSCVASERAFGMNYMGWFRQSAGKQ
REQVAAISQSGDTNYADSVKGRFTISRDNAKNTLYLEMNSLRPE
DTAVYYCYAGPSIGLVISGLGYWGQGTQVTVSS
>L9Ca12
(SEQ ID NO: 166)
EVQLVESGGGLVQPGGSLRLSCAASGFTLENYAIAWFRQAPGKE
REGVSCISSKDSNTYYADSVKGRFTISRDNAKNTVYLQMNSLPE
DTAVYYCAADRLFSCAIIGWDDPPFGSWGQGTQVTVSS
>L9Ca13
(SEQ ID NO: 167)
EVQLVESGGGLVQAGGSLRLSCAASGSIFSINAMAWYRQAPGKQ
REWVARITGDGTTNYADSVKGRFTISRDNAKSTMYLQMNSLKPE
DTAVYYCAARALPLGTDNYWGQGTQVTVSS
>L9Cd12
(SEQ ID NO: 168)
EVQLVESGGGLVQAGGSLRLSCAASGRTFSRYAMGWFRRAPGKE
REFVATISASGMSTYYKDSVKGRFTISRDNAQNTTWLQMNSLKP
EDTAVYYCAAADDFLPRLPSSYTYWGQGTQVTVSS
>L9Cf15
(SEQ ID NO: 169)
EVQLVESGGGLVQAGGSLRLSCVASGSTSSINAMGWYRQTPGKQ
REFLAVIENIGNTRYADSVLGRFTISRDNAKNTVYLQMNSLKPE
DTAVYYCNANARTTPTSISTAWFWGQGTQVTVSS
>L9Cm18
(SEQ ID NO: 170)
EVQLVESGGGLVQAGGSLRLSCAASGFTFDDYDIGWFRQAPGKE
REAVSRISNRDGSTYYLDSVRGRFTASSDNAKNAVYLQMNNLKP
EDTAVYYCAATTALVGMLLPAYEYDYWGQGTQVTVSS
>L9Co22
(SEQ ID NO: 171)
EVQLVESGGGLVQPGGSMRPSCTGSGFTLDDYAIGWFRQAPGKE
REGVSCISSTDSRTYYPDSVEGRFTISRDNAKNTVYLQMNSLKP
EDTAVYYCATDVLECNYPGGTYYPLAFGSWGQGTQVTVSS
>L9Cp5
(SEQ ID NO: 172)
EVQLVESGGGLVQAGGSLRLSCAASGRTFSSCAMGWFRQAPGKE
REIVAAISWRGGSTWYADSVKGRFTISRDNAKNTVYLQMSNLKP
EDTAVYYCAADETSGSYYYTVDIDEYDYWGQGTQVTVSS
>L9Cpl3
(SEQ ID NO: 173)
EVQLVESGGGLVQPGGSLRLSCAASGTAFSITSMGWYRQASGEQ
RELVARIDSDGSTNYADSVKGRFTISRDNAKNTVYLQMNSLKPE
DTAVYYCNAKKWILETYWGQGTQVTVSS
>L1719A9
(SEQ ID NO: 174)
EVQLVESGGGLVQAGDSLRLSCAASGRTFSFDARGWFRQAPGKE
REFVAAINWSGSDTHYADSVKGRFTISRDNAKNTLYLQMNSLKP
EDTAVYYCAAAFDYSGSYNSVGLWDYWGQGTQVTVSS
>L1719A11
(SEQ ID NO: 175)
EVQLVESGGGLVQAGDSLRLSCAASGRTFSFDAKGWFRQAPGKE
REFVAAINWSGSDTHYADSVKGRFTISRDNAKNTAYLQMNSLKP
EDTAVYYCAAAFDYSGSYDSVGLWDYWGQGTQVTVSS
>L1719A12
(SEQ ID NO: 176)
EVQLVESGGGLVQAGDSLRLSCAASGRTFSFDARGWFRQAPGKE
REFVAAINWSGSDTHYADSVKGRFTISRDNAKNTAYLQMNSLKP
EDTAVYYCAAAFDYSGSYASVGLWDYWGQGTQVTVSS
>L1719B12
(SEQ ID NO: 177)
EVQLVESGGGLVQAGDSLRLSCAASGRTFSFDARGWFRQAPGKE
REFVAAINWCGSDAHYADSVKGRFTISRDNAKNTAYLQMNSLKP
EDTAVYFCAAAFDYSGSYNSVGLWDYWGQGTQVTVSS
>L1719C1
(SEQ ID NO: 178)
EVQLVESGGGLVRHGDSLRLSCAASRRTTSVYATGWFRQAPGKE
REFVAAISWSGGYTNYADSVKGRFTISRDNARNTVFLQMNSLKP
EDTAVYYCAADRNPYRGSNNANAYDYWGQGTQVTVSS
>L1719D10
(SEQ ID NO: 179)
EVQLVESGGGLVQAGDSLRI+CAASGRTFSFDARGWFRQAPGKE
REFVAAINWSGSDTHYADSVKGRFTISRDNAKNTVYLQMNSLKP
EDTAVYYCAAAFDYSGSYDSVGLWDYWGQGTQVTVSS
>L1719E1
(SEQ ID NO: 180)
EVQLVESGGGLVRHGDSLRLSCLVSGRTTSVYATGWFRQAPGKE
REFVAAISWSGGYTNYADSVKGRFTISRDNAKNTVFLQMNSLKP
EDTAVYYCAADRNPYRGSNNANAYDYWGQGTQVTVSS
>L1719E11
(SEQ ID NO: 181)
EVQLVESGGGWVQAGDSLRLSCAASGRTFSFDAKGWFRQAPGKE
REFVAAINWSGSDTHYADSVKGRFTISRDNAKNTVYLQMNSLKP
EDTAVYYCAAAFDYSGSYNSVGLWDYWGQGTQVTVSS
>L1719E12
(SEQ ID NO: 182)
EVQLVESGGGWVQAGDSLRLSCAASGRTFSFDAKGWFRQAPGKE
REFVAAINWSGSDTHYADSVKGRFTISRDNAKNTVYLQMNSLKP
EDTAVYYCAAAFDYSGNYNSVGLWDYWGQGTQVTVSS
>L1719F11
(SEQ ID NO: 183)
EVQLVESGGGLVQAGDSLRLSCAASGRTFSFDARGWFRQAPGKE
REFVAAINWSGSDAHYADSVKGRFTISRDNAKNTAYLQMNSLKP
EDTAVYFCAAAFDYSGSYNSVGLWDYWGQGTQVTVSS
>L1719H9
(SEQ ID NO: 184)
EVQLVESGGGLVQAGDSLRLSCAASGRSFSFDARGWFRQAPGKE
REFVAAINWSGSDTHYADSVKGRFTISRDNAKNTAYLQMNSLKP
EDTAVYYCAAAFDYSGSYNSVGLWDYWGQGTQVTVSS
>L1719H10
(SEQ ID NO: 185)
EVQLVESGGGLVQAGDSLRLSCAASGRTFSFDARGWFRQAPGKE
REFVAAINWSGSDAHYADSVKGRFTISRDNAKNTAYLQMNSLKP
EDTAAYFCAAAFDYSGSYNSVGLWDYWGQGTQVTVSS
>L1720C1
(SEQ ID NO: 186)
EVQLVESGGGLVRHGDSLRLSCAASRRTTSVYATGWFRQAPGKE
REFVAAISWSGGYTNYADSVKGRFTISRDNAKDTVFLQMNSLKP
EDTAVYYCAADRNPYRGSNNANAYDYWGQGTQVTVSS
>L1720E4
(SEQ ID NO: 187)
EVQLVESGGGLVRHGDSLRLSCLVSRRTTSVYATGWFRQAPGKE
REFVAAISWSGGYTNYADSVKGRFTISRDNAKNTVFLQMNSLKP
EDTAVYYCAADQNPYRGSNNANAYDYWGQGTQVTVSS
>L1721A3
(SEQ ID NO: 188)
EVQLVESGGGLVRHGDSLRLSCAASRRTTSVYATGWFRQAPGKE
REFVAAISWSGGYTNYADSVKGRFTISRDNAKNTVFLQMNSLKP
EDTAVYYCAADRNPYRGSNNANAYDYWGQGTQVTVSS
>L1721A5
(SEQ ID NO: 189)
EVQLVESGGGLVQAGDSLRLSCAASGRTFSFDAKGWFRQAPGKE
REFVAAINWSGSDTHYADSVKGRFTISRDNAKNTVYLQMNSLKP
EDTAVYYCAAAFDYSGSYDSVGLWDYWGQGTQVTVSS
>L1721A8
(SEQ ID NO: 190)
EVQLVESGGGLVRHGDSLRLSCLVSRRTTSVYATGWFRQAPGKE
REFVAAISWSGGYTNYADSVKGRFTISRDNARNTVFLQMNSLKP
EDTAVYYCAADRNPYRGSNNANAYDYWGQGTQVTVSS
>L1721H4
(SEQ ID NO: 191)
EVQLVESGGGLVQPGGSLRLSCAASGFTLDYYAIGWFRQAPGKE
REGVSCVSSSDGSTYYADSVKGRFTISRDNAENTVYLQMNSLKP
EDTAVYYCATEQSCTGSSWSGPGQGTRVTVSS
>L1723A9
(SEQ ID NO: 192)
EVQLVESGGGLVQAGDSLRLSCAASKRTTSAYATGWFRQAPGKE
REFVAAISYSGAYTNYADSVKGRFTISRDNAKNTVYLQMNSLKP
EDTAVYYCAADRNPYRGSNNANAYDYWGQGTQVTVSS
>L1723A10
(SEQ ID NO: 193)
EVQLVESGGGLVQAGDSLRLSCAASGRTFSFDARGWFRQAPGKE
REFVAAINWSGSDAHYADSVKGRFTISRDNAKNTAYLRMNSLKP
EDTAVYFCAAAFDYSGSYNSVGLWDYWGQGTQVTVSS
>L1723A11
(SEQ ID NO: 194)
EVQLVESGGGLVQAGGSLRLSCRASGNTGNVNAMGWYRQAPGKQ
RELVAAVSGGGKTNYADSVKGRFTISIDNAKIIVYLQMNNLKPE
DTAVYYCNAGVMGTSGSYYLGEDNWGQGTQVTVSS
>L1723E5
(SEQ ID NO: 195)
EVQLVESGGGLVRHGDSLRLSCLVSRRTTSVYATGWFRQAPGKE
REFVAAISWSGGYTNYADSVKGRFTISRDNAKNTVFLQMNSLKP
EDTAVYYCAADRNPYRGSNNANAYDYWGQGTQVTVSS
>L2319G7
(SEQ ID NO: 196)
EVQLVESGEGLVQAGDSLRLSGAASGRTISSYAMGWFRQAPGKE
REFVAAVSMSGGRTLYADSVKGRFTISKDNTENTVYLQMNSLKP
EDTAVYYCAAKVVGYFDVALYAQERDYNYWGQGTQVTVSS
>L2319G11
(SEQ ID NO: 197)
EVQLVESGGGLVQPGGSLRLSCAASGSIFSFNAMAWYRQAPGKQ
RELVAAISSGGSTNYADSVKGRFTISRDNAKNTVYLQMNSLKPE
DTADYYCNLWHTDFVSFYDYWGQGTQVTVSS
>L2319H7
(SEQ ID NO: 198)
EVQLVESGGGLVQAGGSLRLSCAASGRTFSDDLISWFRQAPGKE
RDFVAAVTFYGEDTYYADSVKGRFTISRDNAKNTVYLQMDRLKS
EDTAVYHCASSSRATVIQNPLGYDYWGQGTQVTVSS
>L2320E9
(SEQ ID NO: 199)
EV*LVESGGGLVQAGGSLRLSGAASGRTFSNDLISWFRQAPGKE
RDFVAGVAFYGDDTYYADSVKGRFTISRENAKNRVYLQMDNLKS
EDTGVYYCASSSRASVIQNPLGYDYWGQGTKVTVSS
>L2320F9
(SEQ ID NO: 200)
EVQLVESGGGLVQAGGSLRLSCAASGRTFSNDLISWFRQAPGEE
RDFVAAVAFYGDDTYYADSVKGRFTISRDNAKNTVYLQMDNLKS
EDTAVYYCASSSRATVIQNPLGYDYWGQGTQVTVSS
>L2321B7
(SEQ ID NO: 201)
EVQLVESGGGLVQAGDSLRLSCAASGRTISSYAMGWFRQAPGKE
REFVAAVSMSGGRTLYADSVKGRFTISKDNTENTVYLQMNSLKP
EDTAVYYCAAKWGYFDVALYAQERDYNYWGQGTQVTVSS
>L2321H6
(SEQ ID NO: 202)
EVQLVESGGGLVQAGGSLRLACAASGRTFSDYAVGWFRQAPGKE
RQIVAAISWGGGDTYYTNSVKGRFTISRDNTRNTVYLQMSSLKP
EDTAVYYCAANRNRRGVVSAPSSYDYWGQGTQVTVSS
>VLP_A14
(SEQ ID NO: 203)
EVQLVESGGGLVQAGGSLRLSCAASGFSFNDYAIGWFRQTPGKE
REGVSCLSSSGMAHYADSVKGRFTIAYDNAKNTVYLQMNSLKPE
DTAVYHCATHPFRCGNWRTVMGSWGQGTQVTVSS
>VLP_B9
(SEQ ID NO: 204)
EVQLVESGGGLVQAGGSLRLSCETSGFTFDDYGIGWFRQAPGKG
REGISCISGSGIAHYGDSVKGRFTISSDNAKNTVYLQMNSLKPE
DTGLYYCATTPFRCGNWRTTMGSWGQGTQVTVSS
>VLP3_B21
(SEQ ID NO: 205)
EVQLVESGGGLVQAGGSLRLSCEASTSMFSIRAATWYRQAPGKQ
RELVANIDSEGTTGYSDSVKGRFTISRDNTKKTVYLQMNSLKPE
DTAVYSCNAVVTYNMLVYDSWGQGTQVTVSS

TABLE 3

| | |
|---|---|
| L81H9: | 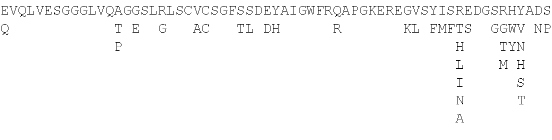 |
| | 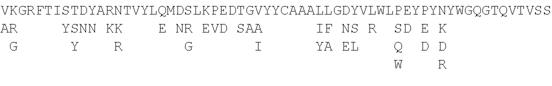 |
| L91B5: | 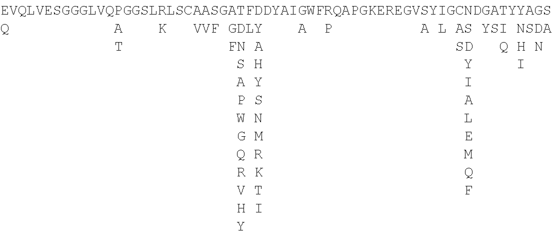 |
| | 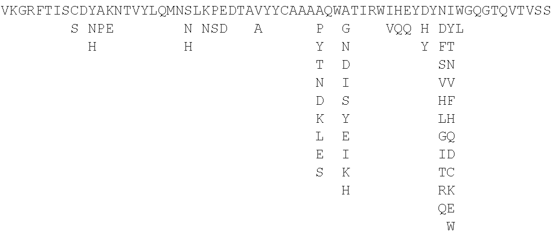 |
| L94D4: | 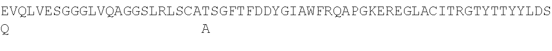 |
| | 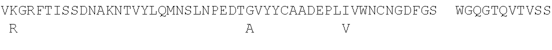 |
| L93E3: | 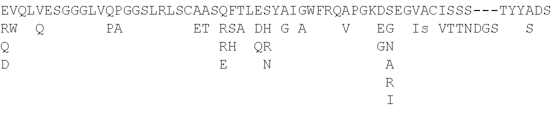 |
| | 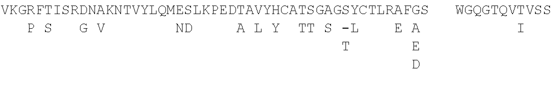 |
| L91F10: | 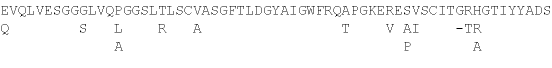 |
| | 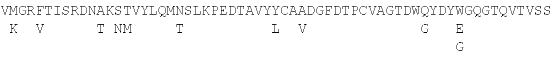 |
| L8Cj3: | 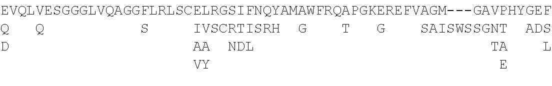 |
| | 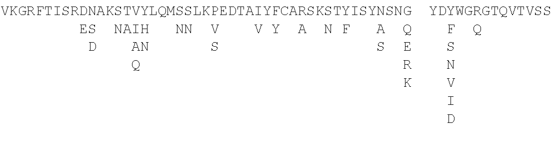 |
| L92E7: | 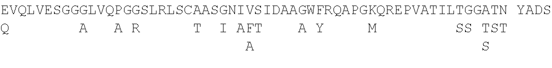 |
| | 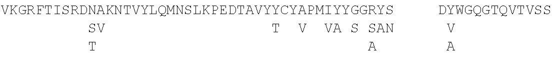 |

Representative sequences of the 7 families of table 2 are shown next to the clone name and underneath are all deviations that have been found for a particular amino acid residue.
The CDRs are highlighted in grey.
(Base sequences disclosed as SEQ ID NOS 28, 37, 47, 55, 289, 207 and 74, respectively, in order of appearance, and the corresponding consensus sequences disclosed as SEQ ID NOS 285-288 and 290-292, respectively, in order of appearance)

TABLE 4
Competition between an excess of unlabeled VHH (columns) and a labeled VHH that was added in a non saturating amount.

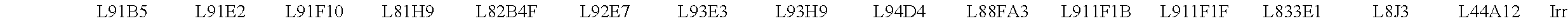

Competition on gp140UG37

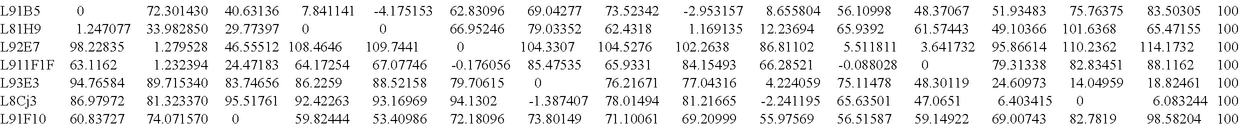

Competition on gp120IIIB

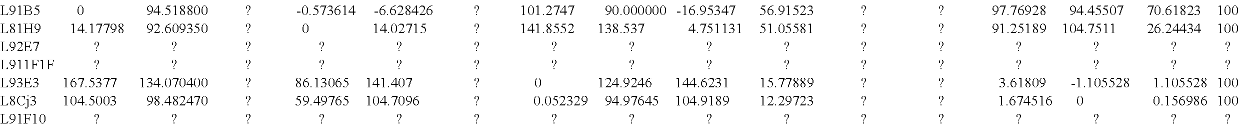

Competition on gp140CN54

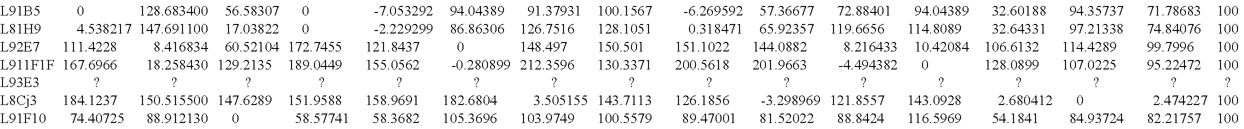

The values are expressed in percentages.
The value of the competition with itself was determined as 0% and the competition with an irrelevant VHH (irr) as 100%.
Competition for the same epitope was determined as a value below 40% (dark grey), possible competition as a value between 40 and 75% (grey) and no competition, and thus no overlapping epitope, as a value above 75% (light grey).
• means that the combination is not tested, due to limited binding of the VHH to the antigen.

TABLE 5
Neutralization data for the L92E7 mutants.

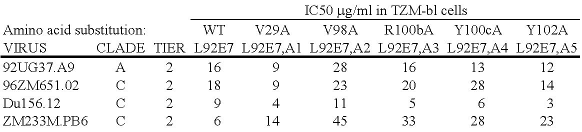

IC50 values from wild type (WT) L92E7 and 5 mutants against various HIV-1 strains given in μg/ml.
Values between 1 and 10 μg/ml are marked in medium grey and between 10 and 50 μg/ml in light grey.

TABLE 6
Maturation of VHH sequence and the future mutations

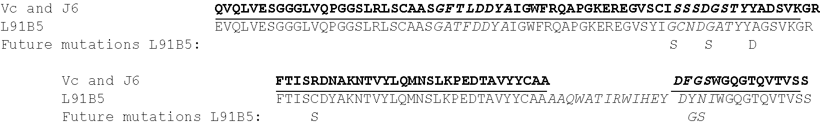

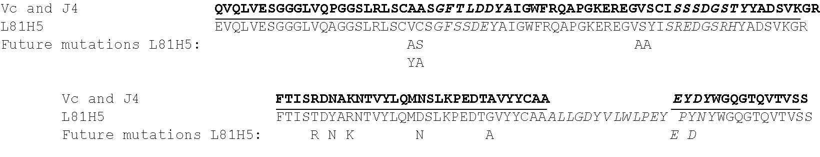

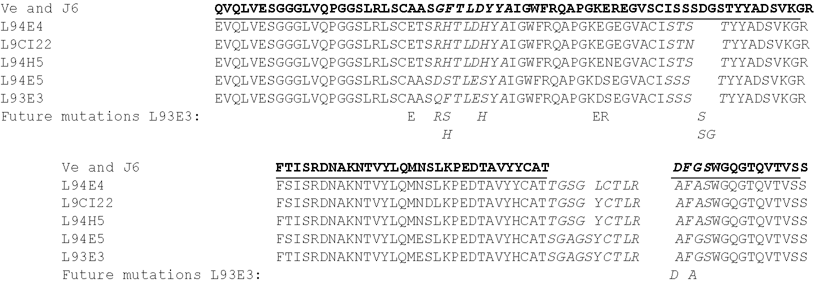

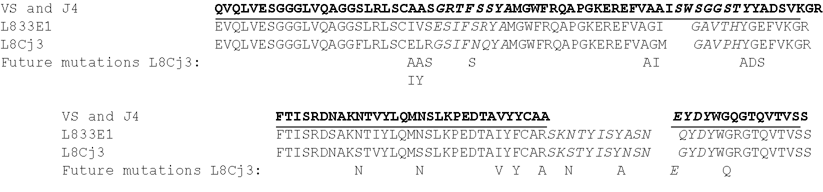

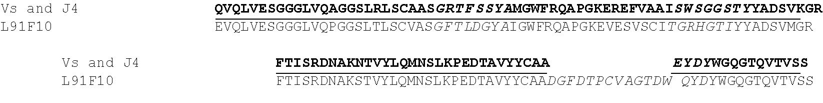

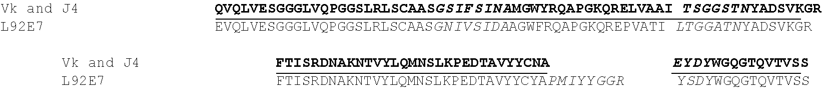

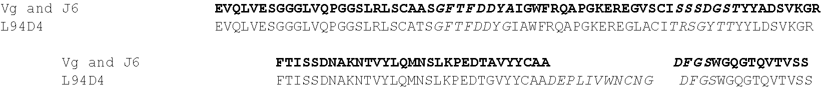

Maturation of the 7 most important VHH (families) of table 2.
The top sequence, in bolt, is the germ line sequence (V and J genes) from which the VHH is derived.
Underneath the germ line sequences are the sequences from the VHH clones and at the bottom, the mutations that will be made for improvement of the VHH.
The CDRs as defined in this invention are marked in Italic.
In light grey are the framework residues that deviate from the germ line and in dark grey the CDR residues.
(Base sequences disclosed as SEQ ID NOS 293, 37, 295, 28, 297, 54, 248, 298-299, 55, 301-303, 301, 64, 306, 74, 308 and 47, respectively, in order of appearance, and the corresponding consensus sequences disclosed as SEQ ID NOS 294, 296, 300, 304-305, 307 and 309, respectively, in order of appearance)

TABLE 7
Biheads
7A Combinations of VHH that will be linked in bi-head constructs to
generate bispecific antibodies. The first column gives all possibilities for
VHH at the N-terminus, the second row gives all possible linker lengths
and the third column gives all the C-terminal possibilities

| N-terminal VHH | linker | C-terminal VHH |
|---|---|---|
| L8Cj3 | 5GS | L8Cj3 |
| L93E3 | 10GS | L93E3 |
| L833E1 | 15GS | L833E1 |
| L92E7 | 20GS | L92E7 |
| L911F1F | 25GS | L911F1F |
| L91B5 | 30GS | L91B5 |
| L91B5 | 30GS | L91B5 |
| L91F10 | 35GS | L91F10 |
| L81H9 | | L81H9 |
| L94D4 | | L94D4 |
| A12 | | A12 |

TABLE 7B
Combinations of VHH that have been linked in bi-head constructs.

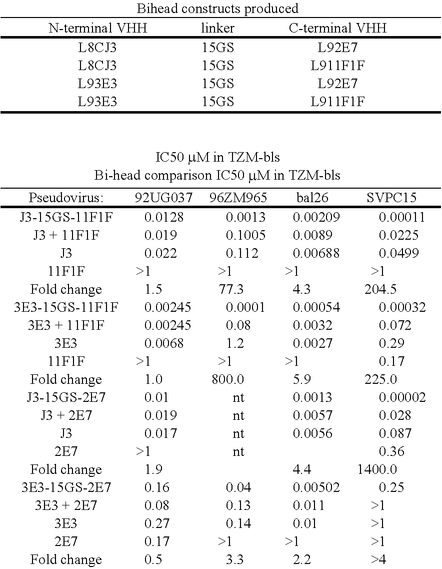

IC50 values in μM are given for purified bi-head constructs, individual VHH and molar mixes of the bi-head components.
Fold increase in potency for the bi-head constructs was calculated relative to the molar mix of the component VHH.
Fold change of 1-2 is coloured light grey, 2-10 is coloured medium grey, 10-100 is coloured darker grey and more than 100 is coloured dark grey.
Nt means not tested.

TABLE 8
Antibody kinetics.The binding kinetics of three sCD4 constructs. six mAbs and ten VHH against four
different HIV-1 envelope proteins as measured by SPR. No fit indicates that the acquired data did
not fit well to the models used and thus no reliable binding constants could be calculated.

| | | | ENVELOPE | | | | | | |
|---|---|---|---|---|---|---|---|---|---|
| | | | CA018 | gp140/420 | (A/G) | IIIB | gp120 | (B) | UG037 |
| | | | kd (1/s) | ka (1/Ms) | KD (M) | kd (1/s) | ka (1/Ms) | KD (M) | kd (1/s) |
| ANTIBODY | CD4 | sCD4 (2-D) | 3.80E−01 | 2.02E+05 | 9.16E−07 | 6.00E−05 | 6.37E+04 | 9.42E−10 | 2.49E−04 |
| | | sCD4 4-D | 6.75E−03 | 8.60E+04 | 7.85E−08 | 1.12E−04 | 1.30E+05 | 8.60E−10 | 3.72E−01 |
| | | sCD4 (full) | 4.17E−04 | 4.24E+04 | 9.84E−09 | 2.77E−04 | 5.48E+05 | 5.05E−10 | no fit |
| | mAbs | b12 | 2.71E−02 | 1.43E+06 | 1.88E−08 | no fit | no fit | no fit | 3.01E−01 |
| | | HJ16 | 3.82E−02 | 3.72E+04 | 1.03E−06 | 1.56E−04 | 5.15E+04 | 3.03E−09 | 3.41E−01 |
| | | 2G12 | 3.42E−01 | 5.00E+05 | 5.37E−07 | 3.14E−04 | 1.65E+05 | 1.91E−09 | 3.49E−01 |
| | | 17b | 6.37E−04 | 5.93E+05 | 1.07E−09 | no fit | no fit | no fit | 1.35E−04 |
| | | 2F5 | 3.99E−01 | 5.82E+04 | 1.01E−07 | no fit | no fit | no fit | 4.22E−01 |
| | | 4E10 | no fit | no fit | no fit | no fit | no fit | no fit | no fit |
| | VHH | A12 | 1.20E−04 | 3.94E+03 | 2.94E−08 | 2.05E−05 | 1.86E+05 | 1.10E−10 | 3.38E−01 |
| | | D7 | 7.90E−03 | 1.17E+03 | 5.54E−07 | 1.46E−03 | 2.60E+05 | 5.62E−09 | 1.30E−04 |
| | | C8 | 1.40E−04 | 2.01E+04 | 6.80E−09 | 5.73E−05 | 1.33E+05 | 4.31E−10 | 5.37E−05 |
| | | L91B5 | 5.27E−03 | 3.39E+05 | 1.56E−08 | 2.60E−04 | 4.97E+05 | 5.17E−10 | 1.33E−06 |
| | | L91H9 | 4.28E−01 | 1.33E+06 | 3.21E−07 | 7.11E−04 | 1.73E+05 | 4.11E−09 | 9.81E−08 |
| | | L91C2 | no fit | no fit | no fit | 2.50E−04 | 1.22E+05 | 2.01E−09 | no fit |
| | | L91F10 | 6.94E−05 | 5.88E+04 | 1.18E−09 | 2.19E−02 | 3.90E+05 | 1.20E−08 | 1.20E−04 |
| | | L92E7 | 1.30E−04 | 1.80E+04 | 7.00E−09 | 7.70E−04 | 1.74E+05 | 4.42E−09 | 2.50E−04 |
| | | L82B4F | 2.98E−03 | 8.50E+02 | 3.50E−06 | 7.34E−04 | 2.30E+07 | 3.15E−11 | 3.25E−08 |
| | | L8Cj3 | 3.98E−05 | 4.49E+03 | 8.86E−09 | 3.27E−06 | 1.19E+04 | 2.28E−11 | 5.48E−08 |

| | | | ENVELOPE | | | | |
|---|---|---|---|---|---|---|---|
| | | | gp140/420 | (A) | CN54 | gp140/420 | (C) |
| | | | ka (1/Ms) | KD (M) | kd (1/s) | ka (1/Ms) | KD (M) |
| ANTIBODY | CD4 | sCD4 (2-D) | 6.25E+03 | 3.98E−08 | 4.37E−01 | 1.29E+04 | 6.10E−06 |
| | | sCD4 4-D | 4.03E+06 | 2.11E−08 | 6.90E−05 | 7.10E+04 | 9.72E−10 |
| | | sCD4 (full) | no fit | no fit | no fit | no fit | no fit |
| | mAbs | b12 | 1.29E+06 | 2.33E−07 | 2.28E−03 | 1.36E+04 | 1.67E−07 |
| | | HJ16 | 4.24E+05 | 8.85E−08 | 4.01E−01 | 5.58E+05 | 3.04E−08 |
| | | 2G12 | 6.95E+05 | 3.93E−07 | no fit | no fit | no fit |
| | | 17b | 5.10E+04 | 2.64E−09 | 1.71E−04 | 2.77E+04 | 6.15E−09 |
| | | 2F5 | 8.09E+04 | 5.85E−08 | no fit | no fit | no fit |
| | | 4E10 | no fit | no fit | no fit | no fit | no fit |
| | VHH | A12 | 2.41E+05 | 1.04E−07 | 4.49E−05 | 2.11E+03 | 2.13E−08 |
| | | D7 | 5.79E+03 | 2.19E−08 | 3.44E−01 | 8.17E+04 | 2.18E−06 |
| | | C8 | 1.91E+04 | 2.81E−09 | 8.78E−05 | 3.00E−04 | 4.18E−09 |
| | | L91B5 | 7.62E+03 | 1.74E−10 | 5.79E−08 | 4.46E+04 | 1.27E−12 |
| | | L91H9 | 9.61E+03 | 1.02E−11 | 1.10E−06 | 1.76E+04 | 6.25E−11 |
| | | L91C2 | no fit | no fit | no fit | no fit | no fit |
| | | L91F10 | 2.93E+04 | 3.98E−09 | 7.12E−07 | 9.23E+03 | 7.71E−11 |
| | | L92E7 | 2.22E+04 | 1.12E−08 | 1.50E−04 | 3.32E+04 | 4.46E−09 |
| | | L82B4F | 1.05E+04 | 3.10E−12 | 5.09E−06 | 5.59E+03 | 3.13E−13 |
| | | L8Cj3 | 7.80E+03 | 7.02E−12 | 2.65E−04 | 1.32E+04 | 1.88E−12 |

TABLE 9
Amino acid sequence of gp120 of Human
immunodeficiency virus type 1 (HXB2)
(SEQ ID NO: 206)

| | |
|---|---|
| MRVKEKYQHL WRWGWRWGTM LLGMLMICSA TEKLWVTVYY | 60 |
| GVPVWKEATT TLFCASDAKA | |
| YDTEVHNVWA THACVPTDPN PQEVVLVNVT ENFNMWKNDM | 120 |
| VEQMHEDIIS LWDQSLKPCV | |
| KLTPLCVSLK CTDLKNDTNT NSSSGRMIME KGEIKNCSFN | 180 |
| ISTSIRGKVQ KEYAFFYKLD | |
| IIPIDNDTTS YKLTSCNTSV ITQACPKVSF EPIPIHYCAP | 240 |
| AGFAILKCNN KTFNGTGPCT | |
| NVSTVQCTHG IRPVVSTQLL LNGSLAEEEV VIRSVNFTDN | 300 |
| AKTIIVQLNT SVEINCTRPN | |
| NNTRKRIRIQ RGPGRAFVTI GKIGNMRQAH CNISRAKWNN | 360 |
| TLKQIASKLR EQFGNNKTII | |
| FKQSSGGDPE IVTHSFNCGG EFFYCNSTQL FNSTWFNSTW | 420 |
| STEGSNNTEG SDTITLPCRI | |
| KQIINMWQKV GKPMYAPPIS GQIRCSSNIT GLLLTRDGGN | 480 |
| SNNESEIFRP GGGDMRDNWR | |
| SELYKYKVVK IEPLGVAPTK AKRRVVQREK R | 511 |

TABLE 10
(A) Neutralization ability of wild type J3 and family
member mutants against various HIV-1 strains.

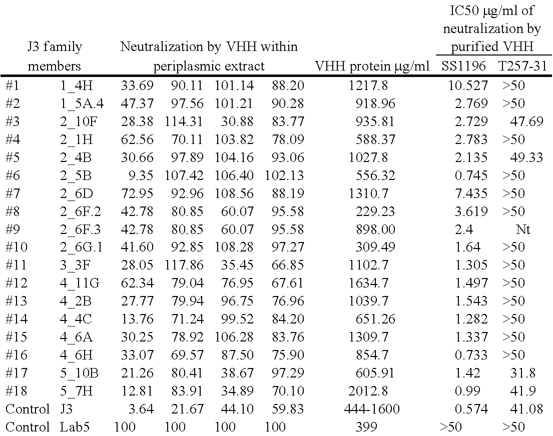

(B) Neutralization ability of wild type 3e3 and family
member mutants against various HIV-1 strains.

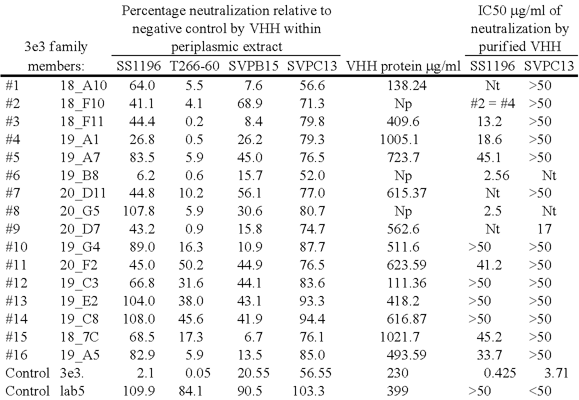

For purified VHH IC50 values in μg/ml less than 1 are marked in dark grey, values between 1 and 10 μg/ml are marked in medium grey and values between 10 and 50 μg/ml in light grey.
Nt = not tested.

TABLE 11A
Sequences of J3 family members

>L8Cj3
(SEQ ID NO: 207)
EVQLVESGGGLVQAGGFLRLSCELRGSIFNQYAMAWFRQAPGKEREF
VAGMGAVPHYGEFVKGRFTISRDNAKSTVYLQMSSLKPEDTATYFCA
RSKSTYISYNSNGYDYWGRGTQVTVSS
>L833E1
(SEQ ID NO: 208)
EVQLVESGGGLVQAGGSLRLSCIVSESIFSRYAMGWFRQAPGKEREF
VAGIGAVTHYGEFVKGRFTISRDSAKNTIYLQMSSLKPEDTAIYFCA
RSKNTYISYASNQYDVWGRGTQVIVSS
>L8i5
(SEQ ID NO: 209)
EVQLVESGGGLVQAGGFLRLSCEPRGSIFNQYAMAWFRQAPGKEREF
VAGMGAVAHYGEFVKGRFTISRDNAKNTAYLQMNSLKPEDTAIYFCA
RSKSTYISYNSNGYDYWGRGTQVTVSS
>J3r (for improved expression yield in yeast)
(SEQ ID NO: 210)
DVQLQESGGGLVQAGGSLRLSCELRGSIFNQYAMAWFRQAPGKEREF
VAGMGAVPHYGEFVKGRFTISRDNAKSTVYLQMNSLKPEDTAIYFCA
RSKSTYISYNSNGYDYWGQGTQVTVSS
>1_4H (family approach)
(SEQ ID NO: 211)
EVQLVESGGGLVQAGGFLRLSCVLRGSIFNQYAMAWFRQAPGKEREF
VAGMGAVPHYGEFVKGRFTISRDNAKSTVYLQMNSLKPEDTAIYFCA
RSKSTYISYNSNQYDFWGQGTQVTVSS
>2_1H (family approach)
(SEQ ID NO: 212)
EVQLVESGGGLVQAGGSLRLSCVLRGSDFSRYAMGWFRQAPGKEREF
VAGMGAVAHYGEFVKGRFTISRDNAKNAQYLQMNSLKPEDTAIYFCA
RSKSTYISYNSNEYDYWGRGTQVTVSS
>2_4B (family approach)
(SEQ ID NO: 213)
EvQLVESGGGLVQAGGSLRLSCELRGSIFSQYAMGWFRQAPGKEREF
VAGMGAVAHYGEFVKGRFTISRDDAKNTINLQMNSLKPEDTATYFCA
RSKSTYISYNSNQYDFWGQGTQVTVSS
>2_5B (family approach)
(SEQ ID NO: 214)
EVQLVESGGGLVQAGGSLRLSCVLRGSDFSRYAMGWFRQAPGKEREF
VAGMGAVAHYGEFVKGRFTISRDNAKNAQYLQMNSLKPEDTAIYFCA
RSKSTYISYNSNQYDIWGQGTQVTVSS
>2_6D (family approach)
(SEQ ID NO: 215)
EVQLVESGGGLVQAGGSLRLSCVVSENIFSRYAMGWFRQAPGKEREF
VAGIGANTHYGEFVKGRFTISRDNAENTIYLQMNSLKPEDTAIYFCA
RSKNTYISYSSNQYDDWGQGTQVTVSS
>2_10F (family approach)
(SEQ ID NO: 216)
EVQLVESGGGLVQAGGSLRLSCELRGSIFSQYAMGWFRQAPGKEREF
VAGMGAVAHYGEFVKGRFTISRDDAKNTINLQMNSLKPEDTAIYFCA
RSKSTYISYNSNGYDVWGQGTQVTVSS
>3_3F (family approach)
(SEQ ID NO: 217)
EVQLVESGGGLVQAGGFLRLSCELRGSIFNQYAMAWFRQAPGKEREF
VAGMGAVPHYGEFVKGRFTISRDNAKNSVYLQMNSLKVEDTAIYFCA
RSKSTYISYNSNGYDSWGRGTQVTVSS
>4_2B (family approach)
(SEQ ID NO: 218)
EVQLVESGGGLVQAGGFLRLSCELRGSIFNQYAMAWFRQAPGKEREF
VAGMGAVPHYGEFVKGRFTISRDNAKNTVYLQMNSLKPEDTAIYFCA
RSKSTYISYNSNRYDYWGRGTQVTVSS
>4_4C (family approach)
(SEQ ID NO: 219)
EVQLVESGGGLVQAGGFLRLSCELRGSIFNQYAMAWFRQAPGKEREF
VAGMGAVPHYGEFVKGRFTISRDNAKSTVYLQMNSLKPEDTAJYFCA
RSKSTYISYNSNQYDYWGQGTQVTVSS
>4_6A (family approach)
(SEQ ID NO: 220)
EVQLVESGGGLVQAGGFLRLSCELRGSIFNQYAMAWFRQTPGKEREF
VAGMGAVPHYGEFVKGRFTISRDNAKNITYLQMNSLKPEDTAIYFCA
RSKSTYISYNSNRYDYWGRGTQVTVSS
>4_6H (family approach)
(SEQ ID NO: 221)
EVQLVESGGGLVQAGGSLRLSCELRGSIFSRYAMGWFRQAPGKEREF
VAGMGAVEHYGEFVKGRFTISRDNAKNTVHLQMNSLKSEDTAIYFCA
RSKSTYISYNSNRYDNWGRGTQVTVSS
>4_11G (family approach)
(SEQ ID NO: 222)
EVQLVESGGGLVQAGGSLRLSCVVSGSIFNQHAMGWFRQAPGKGREF
VSGLSGTEHYGDLVKGRFTISRDNAKNTIYLQMNNLKPEDTAIYFCA
RSKNTFISYSSNKYDFWGQGTQVIVSS
>5_7H (family approach)
(SEQ ID NO: 223)
EVQLVESGGGLVQAGGFLRLSCELRGSIFNQYAMAWFRQAPGKEREF
VAGMGAVPHYGEFVKGRFTISRDNAKNTVYLQMNSLKPEDTAIYFCA
RSKSTYISYNSNGYDYWGRGTQVTVSS
>5_10B (family approach)
(SEQ ID NO: 224)
EVQLVESGGGLVQAGGSLRLSCELRGSIFSQYAMGWFRQAPGKEREF
VAGMGAVAHYGEFVKGRFTISREDAKNTINLQMNSLKPEDTAIYFCA
RSKSTYISYNSNGYDFWGRGTQVTVSS
>9_4F (family approach)
(SEQ ID NO: 225)
EVQLVESGGGLVQAGGSLRLSCVVSGSIFSQHAMGWFRQAPGKGREF
VSGISGTTHYGDLVKGRFTISRDNAKNTIYLQMNSLKPEDTAIYFCA
RSKNTYISYSSNGYDIWGRGTQVTVSS
>J3_1 (site directed mutagenesis)
(SEQ ID NO: 226)
EVQLVESGGGLVQAGGFLRLSCAASGSIFNQYAMAWFRQAPGKEREF
VAGMGAVPHYGEFVKGRFTISRDNAKSTVYLQMSSLKPEDTAIYFCA
RSKSTYISYNSNGYDYWGRGTQVTVSS
>J3_2 (site directed mutagenesis)
(SEQ ID NO: 227)
EVQLVESGGGLVQAGGFLRLSCELRGSIFSQYAMAWFRQAPGKEREF
VAGMGAVPHYGEFVKGRFTISRDNAKSTVYLQMSSLKPEDTAIYFCA
RSKSTYISYNSNGYDYWGRGTQVTVSS
>J3_3 (site directed mutagenesis)
(SEQ ID NO: 228)
EVQLVESGGGLINAGGFLRLSCELRGSIFNQYAMAWFRQAPGKEREF
VAAIGAVPHYGEFVKGRFTISRDNAKSTVYLQMSSLKPEDTAIYFCA
RSKSTYISYNSNGYDYWGRGTQVTVSS
>J3_4 (site directed mutagenesis)
(SEQ ID NO: 229)
EVQLVESGGGLVQAGGFLRLSCELRGSIFNQYAMAWFRQAPGKEREF
VAGMGAVPHYADSVKGRFTISRDNAKSTVYLQMSSLKPEDTAIYFCA
RSKSTYISYNSNGYDYWGRGTQVTVSS
>J3_5 (site directed mutagenesis)
(SEQ ID NO: 230)
EVQLVESGGGLVQAGGFLRLSCELRGSIFNQYAMAWFRQAPGKEREF
VAGMGAVPHYGEFVKGRFTISRDNAKSTVYLQMSSLKPEDTAVYFCA
RSKSTYISYNSNGYDYWGRGTQVTVSS
>J3_6 (site directed mutagenesis)
(SEQ ID NO: 231)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYYCA
RSKSTYISYNSNGYDYWGRGTQVITSS
>J3_7 (site directed mutagenesis)
(SEQ ID NO: 232)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYFCA
ASKSTYISYNSNGYDYWGRGTQVITSS
>J3_8 (site directed mutagenesis)
(SEQ ID NO: 233)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYFCA
RSKNTYISYNSNGYDYWGRGTQVTVSS
>J3_9 (site directed mutagenesis)
(SEQ ID NO: 234)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYFCA
RSKSTYISYASNGYDYWGRGTQVTVSS
>J3_10 (site directed mutagenesis)
(SEQ ID NO: 235)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYFCA
RSKSTYISYNSNEYDYWGRGTQVTVSS
>J3_11 (site directed mutagenesis)
(SEQ ID NO: 236)
EVQLVESGGGLVQAGGFLRLSCELRGSIFNQYAMAWFRQAPGKEREF
VAGMGAVPHYGEFVKGRFTISRDNAKSTVYLQMSSLKPEDTAIYFCA
RSKSTYISYNSNGYDYWGQGTQVTVSS
>J3_12 (site directed mutagenesis)
(SEQ ID NO: 237)
EVQLVESGGGLVQAGGFLRLSCIYRGSIFNQYAMAWFRQAPGKEREF
VAGMGAVPHYGEFVKGRFTISRDNAKSTVYLQMSSLKPEDTAIYFCA
RSKSTYISYNSNGYDYWGRGTQVTVSS
>J3_13 (site directed mutagenesis)
(SEQ ID NO: 238)
EVQLVESGGGLVQAGGFLRLSCELRGRTFNQYAMAWFRQAPGKEREF
VAGMGAVPHYGEFVKGRFTISRDNAKSTVYLQMSSLKPEDTAIYFCA
RSKSTYISYNSNGYDYWGRGTQVTVSS
>J3_CDR2_fill_1 (site directed mutagenesis)
(SEQ ID NO: 239)
EVQLVESGGGLVQAGGFLRLSCELRGSIFNQYAMAWFRQAPGKEREF
VAGMSGAVPHYGEFVKGRFTISRDNAKSTVYLQMSSLKPEDTAIYFC
ARSKSTYISYNSNGYDYWGRGTQVTVSS
>J3_CDR2_fill_2 (site directed mutagenesis)
(SEQ ID NO: 240)
EVQLVESGGGLVQAGGFLRLSCELRGSIFNQYAMAWFRQAPGKEREF
VAGMSWGAVPHYGEFVKGRFTISRDNAKSTVYLQMSSLKPEDTAIYF
CARSKSTYISYNSNGYDYWGRGTQVTVSS
>J3_CDR2_fill_3 (site directed mutagenesis)
(SEQ ID NO: 241)
EVQLVESGGGLVQAGGFLRLSCELRGSIFNQYAMAWFRQAPGKEREF
VAGMSWSGAVPHYGEFVKGRFTISRDNAKSTVYLQMSSLKPEDTAIY
FCARSKSTYISYNSNGYDYWGRGTQVTVSS

TABLE 11B
Sequences of 3E3 family members

>L93E3
(SEQ ID NO: 242)
EVQLVESGGGLVQPGGSLRLSCAASQFTLESYAIGWFRQAPGPMSEG
VACISSSTYYADSVKGRFTISRDNAENTVYLQMESLKPEDTAVYHCA
TSGAGSYCTLRAFGSWGQGTQVTVSS
>3E3r (for improved expression yield in yeast)
(SEQ ID NO: 243)
DVQLQESGGGLVQAGGSLRLSCAASQFTLESYAIGWFRQAPGKDSEG
VACISSSTYYADSVKGRFTISRDNAENTVYLQMNSLKPEDTAVYHCA
TSGAGSYCTLRAFGSWGQGTQVTVSS
>3E3CDR2 (3E3 with a three amino acid,
back to germline, insertion in CDR2)
(SEQ ID NO: 244)
QVQLQESGGGLVQAGGSLRLSCAASQFTLESYAIGWFRQAPGKDSEG
VACISSSDGSTYYADSVKGRFTISRDNAKNTVYLQMNSLKPEDTAVY
HCATSGAGSYCTLRAFGSWGQGTQVTVSS
>L94H5
(SEQ ID NO: 245)
EVQLVESGGGLVQPGGSLRLSCETSRHTLDHYAIGWFRQAPGKENEG
VACISTSTYYADSVKGRFSISRDNAKNTVYLQMNSLKPEDTAVYYCA
TTGSGYCTLRAFASWGQGTQVTVSS
>L9Bf11
(SEQ ID NO: 246)
EVQLVESGGGLVQPGGSLRLSCETSRHTLDHYAIGWFRQAPGEENEG
VACISTSTYYADSVKGRFSISRDNVKNTVYLQMNSLKPEDTAVYYCA
TTGSGYCTLRAFASWGQGTQVTVSS
>L93C3
(SEQ ID NO: 247)
RWQLVESGGGLVQPGGSLRLSCETSRHTLDHYAIGWFRQAPGKGGEG
VACISTNTYYADSVKGRFSISRDNAKNTVYLQMNDLKPEDTAVYHCA
TTGSGYCTLRAFASWGQGTQVTVSS
>L9C122
(SEQ ID NO: 248)
EVQLVESGGGLVQPGGSLRLSCETSRHTLDHYAIGWFRQAPGEEGEG
VACISTNTYYADSVKGRFSISRDNAKNTVYLQMNDLKPEDTAVYHCA
TTGSGYCTLRAFASWGQGTQVTVSS
>L922E2
(SEQ ID NO: 249)
EVQLVESGGGLVQPGGSLRLSCETSRHTLDHYAIAWFRQAPGKEGEG
IACVSTSTYYADSVKGRFSISRGNAKNTVYLQMNDLKPEDTAVYHCA
TTGSGYCTLRAFASWGQGTQVTVSS
>L94E4
(SEQ ID NO: 250)
EVQLVESGGGLVQPGGSLRLSCETSRHTLDHYAIGWFRQAPGEEGEG
VACISTSTYYADSVKGRFSISRDNAKNTVYLQMNSLKPEDTAVYYCA
TTGSGLCTLRAFASWGQGTQVTVSS
>L94E5
(SEQ ID NO: 251)
EVQLVESGGGLVQPGGSLRLSCAASQSTLESYAIGWFRQAPGKDSEG
VACISSSTYYADSVKGRFTISRDNAKNTVYLQMESLKPEDTAVYHCA
TSGAGSYCTLRAFGSWGQGTQVTVSS
>3E3_1 (family approach)
(SEQ ID NO: 252)
EVQLVESGGGLVQPGGSLRLSCETSRHTLDHYAIGWFRQAPGKENEG
VACISTSTYYADSVKGRFSISRDNAKNTVYLQMNSLKPEDTAVYYCA
TTGSGYCTLRAFDSWGQGTQVTVSS
>3E3_2 (family approach)
(SEQ ID NO: 253)
EVQLVESGGGLVPPGGSLRLSCAASQFTLESYAIGWFRQAPGEDSEG
VACISSSTYYADSVKGRFTISRDNAKNTVYLQMESLKPEDTAVYHCA
TSGAGSYCTLRAFASWGQGTQVTVSS
>3E3_3 (family approach)
(SEQ ID NO: 254)
EVQLVESGGGLVQPGGSLRLSCETSRHTLDHYAIAWFRQAPGKEAEG
VACISTSTYYADSVKGRFSISRDNAKNTVYLQMNSLEPEDTAVYHCA
TTGSGYCTLRAFASWGQGTQVTVSS
>3E3_4 (family approach)
(SEQ ID NO: 255)
EVQLVESGGGLVPPGGSLRLSCAASQFTLESYAIGWFRQAPGKDSEG
VACISSSTYYADSVKGRFTISRDNAKNTVYLQMESLKPEDTAVYHCA
TSGAGSYCTLRAFASWGQGTQVTVSS
>3E3_5 (family approach)
(SEQ ID NO: 256)
EVQLVESGGGLVQPGGSLRLSCAASQFTLESYAIGWFRQAPGKDSEG
VACISSSTYYADSVKGRFTISRDNAKNTVYLQMESLKPEDTAVYHCA
TSGAGSYCTLRAFESWGQGTQVTVSS
>3E3_6.1 (family approach)
(SEQ ID NO: 257)
EVQLVESGGGLVQPGGSLRLSCETSRHTLDHYAIGWFRQAPGKEGEG
VACISTSTYYADSVKGRFSISRDNAKNTVYLQMNSLKPEDTAVYYCA
TTGSGYCTLRAFGSWGQGTQVTVSS
>3E3_7 (family approach)
(SEQ ID NO: 258)
EVQLVESGGGLVQPGGSLRLSCAASEFALQNYAIGWFRQAPGKDSEG
VACISSSTYYADSVKGRFTISRDNAKNTVYLQMNSLKPEDTAVYYCA
TSGAGTYCTLRAFASWGQGTQVTVSS
>3E3_8.1 (family approach)
(SEQ ID NO: 259)
EVQLVESGGGLVQPGGSLRLSCETSRHTLDHYAIGWFRQAPGKEGEG
VACISTSTYYADSVKGRFSISRDNAKNTVYLQMNDLKPEDTAVYHCA
TTGSGYCTLRAFESWGQGTQVTVSS
>3E3_8.3 (family approach)
(SEQ ID NO: 260)
EVQLVESGGGLVQPGGSLRLSCETSRHTLDHYAIGWFRQAPGKEGEG
VACISTSTYYADSVKGRFSISRDNAKNTVYLQMNDLKPEDTAVYHCA
TTGSGYCTLREFASWGQGTQVTVSS
>3E3_8.2 (family approach)
(SEQ ID NO: 261)
EVQLVESGGGLVPPGGSLRLSCAASQFTLESYAIGWFRQAPGKDIEG
VACISSSTYYADSVKGRFTISRDNAKNTVYLQMESLKPEDTAVYHCA
TSGAGSYCTLRAFESWGQGTQVTVSS
>3E3_9 (family approach)
(SEQ ID NO: 262)
EVQLVESGGGLVQPGGSLRLSCETSRHTLDHYAIAWFRQAPGKEGEG
VACISTSTYYADSVKGRFSISRDNAKNTVYLQMNDLKPEDTALYHCA
TTGSGYCTLRAFASWGQGTQVTVSS
>3E3_10 (family approach)
(SEQ ID NO: 263)
EVQLVESGGGLVQPGGSLRLSCETSRHTLDHYAIAWFRQAPGKEGEG
IACVSTSTYYADSVKGRFSISRDNAKNTVYLQMNDLKPEDTAVYHCA
TTGSGYCTLRAFDSWGQGTQVTVSS
>3E3_11 (family approach)
(SEQ ID NO: 264)
EVQLVESGGGLVQPGGSLRLSCETSKHTLDHYAIGWFRQAPGKEREG
VSCISTNTYYSDSVKGRFTISRDNAKNTVYLQMNSLEPEDTAVYYCA
TTGSGLCTLRAFASWGQGTQVTVSS
>3E3_12 (family approach)
(SEQ ID NO: 265)
EVQLVESGGGFVQPGGSLRLSCETSRHTLDHYAIGWFRQAPGKEYEG
ISCISTSTYYADSVKGRFTISRDNAKNTVYLQMNSLKPEDTAVYYCA
TTGSGLCTLRAFGSWGQGTQVTVSS
>3E3_13 (family approach)
(SEQ ID NO: 266)
EVQLVESGGGLVQPGGSLRLSCETSRHTLDHYAIGWFRQAPGKEGEG
VACISTSTYYADSVKGRFSISRDNAKNTVYLQMNDLKPEDTAVYHCA
TTGSGLCTLRAFASWGQGTQVTVSS
>3E3_14 (family approach)
(SEQ ID NO: 267)
EVQLVESGGGLVQPGGSLRLSCETSRHTLDHYAIGWFRQAPGKEYEG
ISCISSSTYYADSVKGRFTISRDNAKNTVYLQMNSLKPEDTAVYYCA
TTGSGYCTLRAFESWGQGTQVTVSS
>3E3_15 (family approach)
(SEQ ID NO: 268)
EVQLVESGGGLVQPGGSLRLSCETSRHTLDRYAIGWFRQVPGKEGEG
VACISTSTYYADSVKGRFSISRDNAKNTVYLQMNDLKPEDTAVYHCA
TTGSGYCTLRAFDSWGQGTQVTVSS
>3E3_16 (family approach)
(SEQ ID NO: 269)
EVQLVESGGGLVQPGGSLRLSCETSRHTLDHYAIGWFRQAPGKEAEG
VACISTSTYYADSVKGRFSISRDNAKNTVYLQMNDLKPEDAAVYHCA
TTGSGYCTLRAFASWGQGTQVTVSS

TABLE 12
Anti-HIV activity of VHH-FC constructs
IC50 μM for neutralization of HIV in TZM-bl cells

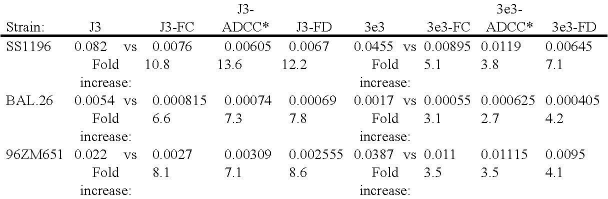

IC50 μg/ml for killing of HIV-infected CD4+ T cells by CD16+ NK cells

| Strain: | J3 (no FC) | J3-FC | J3-ADCC* | J3-FD |
|---|---|---|---|---|
| WEAU | >100 | 17.9 | 9 | >100 |

VHH-FC (hinge and Fc region were from human IgG1) constructs based on J3 and 3E3 were produced and IC50 values in μM calculated against the indicated HIV strains in TZM-bl cell neutralization assays.
IC50 values of greater than 0.01 are coloured white, values between 0.001-0.01 are coloured light grey, those between 0.0001-0.001 are coloured dark grey. Fold increase is shown is calculated relative to the VHH alone.
IC50 values were also calculated for ADCC activity in μg/ml using autologous donor infected CD4+ T cells and NK cells. Fold increase in this case is relative to the VHH-FC.

TABLE 13
SHIV neutralization by L8Cj3
VHH L8Cj3 neutralization activity was assessed in the
indicated SHIV pseudoviruses.

| | | IC₅₀ in TZM-bl |
|---|---|---|
| SHIV | | cells |
| pseudovirus | Clade | L8Cj3 (μg/ml) |
| SHIVsf162p4 | B | 0.0685 |
| SHIVsf162p3 | B | 0.2154 |
| SHIV89.6p | B | 0.4215 |
| SHIV89.6 | B | 0.1528 |
| SHIV1157IPD3N4 | C | 0.01976 |
| SHIV1157IP EL-p | C | 0.006205 |

TABLE 14
Neutralization by VLP1_b9, VLP3_b21, and VLP1_A14 VHH

| | | IC50 values ug/ml | | |
|---|---|---|---|---|
| Subtype | VIRUS: | VLP1_b9 | VLP3_b21 | VLP1_A14 |
| A | 92UG037 | | | |
| A | MS208.A1 | | | |
| A | Q461.e2 | | | |
| A | Q23.17 | | | |
| A | Q842.d12 | | | |
| A | 0330.v4.c3 | | | |
| AC | 3301.v1.c24 | | | |
| AC | 6041.v3.c23 | | | |
| AC | 6540.v4.c1 | | | |
| ACD | 0815.v3.c3 | | | |
| AG | T257-31 | | | |
| AG | T250-4 | | | |
| AG | T266-60 | | | |
| AG | 236-8 | | | |
| AG | 271-11 | | | |
| AG | 242-14 | | | |
| AG | 928-28 | | | |
| AE | R1166.c07 | | | |
| AE | R3265.c06 | | | |
| AE | C2101.c01 | | | |
| CRF01_AE | C1080.c3 | | | |
| CRF01_AE | 620345.c1 | | | |
| B | SS1196 | | | |
| B | Bal26 | | | |
| B | TRJO4551.58 | | | |
| B | AC10.0.29 | | | |
| B | IIIb | | | |
| B | THRO4156.18 | | | |
| B | 6535,3 | | | |
| B | QH0692.42 | | | |
| B | SC422661.8 | | | |
| B | PVO.4 | | | |
| B | TRO.11 | | | |
| B | RHPA4259.7 | | | |
| B | REJO4541.67 | | | |
| B | WITO4160.33 | | | |
| B | CAAN5342.A2 | | | |
| BC | CH038 | | | |
| BC | CH181.12 | | | |
| BC | CH110.2 | | | |
| BC | CH064.20 | | | |
| BC | CH091.9 | | | |
| BC | CNE19 | | | |
| BC | CNE20 | | | |
| BC | CNE21 | | | |
| C | DU172 | | | |
| C | TV1.12 | | | |
| C | Du156.12 | | | |
| C | ZM135M.PL10a | | | |
| C | ZM109F.PB4 | | | |
| C | 96ZM651.02 | | | |
| C | 93MW965.26 | | | |
| C | Du422.1 | | | |
| C | ZM197M.PB7 | | | |
| C | ZM214M.PL15 | | | |
| C | ZM249M.PL1 | | | |
| C (T/F) | Ce0393_C3 | | | |
| C (T/F) | Ce1176_A3 | | | |
| C (T/F) | Ce2010_F5 | | | |
| CD | 6952.v1.c20 | | | |
| CD | 6811.v7.c18 | | | |
| CD | 3817.v2.c59 | | | |
| D | 3016.v5.c45 | | | |
| D | 6405.v4.c34 | | | |
| G | X2131_C1_B5 | | | |
| G | P1981_C5_3 | | | |
| G | X1632_S2_B10 | | | |
| G | X1254_C3 | | | |
| G | X2160_C25 | | | |
| G | X2088_C9 | | | |
| G | X1193_C1 | | | |
| | viruses | 55 | 51 | 51 |
| | neutralized | | | |
| | viruses tested | 71 | 71 | 68 |
| | % | 77 | 72 | 75 |

TABLE 15
Sequences of the VHHs selected from the VLP
libraries VLP_A14, VLP_b9 and VLP3_b21.

| | |
|---|---|
| VLP1 A14: | 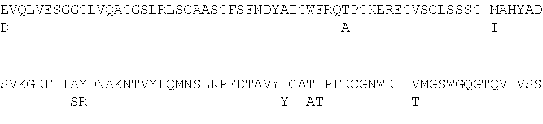 |
| VLP1_b9: | 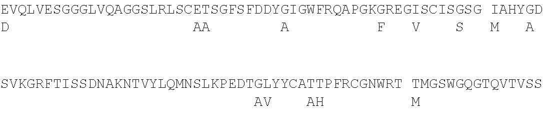 |
| VLP3_b21: | 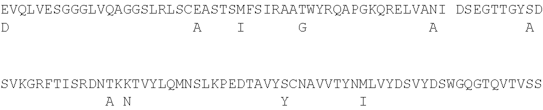 |

Amino acid deviations that have been found for a particular amino acid residue are indicated underneath the amino acid sequence.
The CDR regions are highlighted in grey.]
(Base sequences disclosed as SEQ ID NOS 203-204 and 312, respectively, in order of appearance, and the corresponding consensus sequences disclosed as SEQ ID NOS 310, 311 and 313, respectively, in order of appearance)

1. A broadly neutralizing anti-HIV single heavy chain variable domain antibody (VHH) that neutralizes at least 70% of individual viruses of 5 or more different subgroups of viruses.

2. A broadly neutralizing anti-HIV single heavy chain variable domain antibody (VHH) according to claim 1, which neutralizes at least 75% of individual viruses of at least 5 different subgroups.

3. The heavy chain variable domain antibody of claim 1, wherein said antibody binds nearly exclusively to a CD4 binding site on HIV.

4. The heavy chain variable domain antibody of claim 1, wherein the CDR2 region as defined by amino acid residues 52-58 (according to the Kabat numbering) consists of 5 amino acids.

5. The heavy chain variable domain antibody of claim 1, comprising CDR1, CDR2 and CDR3 amino acid sequences as depicted in table 2, table 3, and/or table 6.

6. The heavy chain variable domain antibody of claim 1, comprising CDR1, CDR2 and CDR3 amino acid sequences as depicted in table 11.

7. The heavy chain variable domain antibody of claim 1, wherein said antibody is fused to an immunoglobulin Fc region or functional part thereof.

8. The heavy chain variable domain antibody of claim 7, wherein the Fc region or functional part thereof is derived from IgGl, IgG2, IgG3, or IgG4.

9. The antibody of claim 7, wherein the Fc region or functional part thereof is human or a humanized lama Fc or functional part thereof.

10. A bi- or multispecific antibody comprising a heavy chain variable domain antibody of claim 1.

11. The bi- or multispecific antibody according to claim 10, wherein the heavy chain variable domain antibodies are non-competing and non-interfering anti-HIV heavy chain variable domain antibodies

12. The bi- or multispecific antibody according to claim 10, wherein at least one heavy chain variable domain antibody is selected from L8Cj3, L93E3, VLP_A14, VLP_B9 and VLP3_B21 and a non-competing and non-interfering anti-HIV heavy chain variable domain antibody is selected from L81H9, L91B5, L94D4 and L91F10 and L92E7.

13. A nucleic acid encoding an antibody of claim 12.

14. A method for producing an antibody, the method comprising expressing the nucleic acid of claim 13 in a relevant cell and recovering the thus produced antibody from the cell.

15. The antibody according to claim 1, for use in diagnostic applications.

16. The antibody of claim 1 for use as a medicament.

17. The antibody of claim 1 for use in a method for treatment of an individual infected with HIV.

18. A microbicide or apheresis device comprising an antibody of claim 1.

19. A pharmaceutical composition comprising an antibody of claim 1.

20. The heavy chain variable domain antibody of claim 7, wherein the Fc region or functional part thereof is derived from IgGl.
